(19) United States
(12) Patent Application Publication
(10) Pub. No.: US 2003/0059826 A1
(43) Pub. Date: Mar. 27, 2003

(54) SOLUBLE COMBINATORIAL LIBRARIES

(76) Inventors: KIM JANDA, SAN DIEGO, CA (US); HAN HYUNSOO, SAN DIEGO, CA (US)

Correspondence Address: THOMAS E. NORTHRUP, ESQ., THE SCRIPPS RESEARCH INSTITUTE, OFFICE OF PATENT COUNSEL, TPC-8, 10550 NORTH TORREY PINES ROAD, LA JOLLA, CA, 92037

(21) Appl. No.: 08/793,015

(22) PCT Filed: Jul. 26, 1995
(86) PCT No.: PCT/US95/09614

(51) Int. Cl. G01N 33/53; G01N 33/543; A61K 38/00; A61K 38/16

(52) U.S. Cl. 435/7.1; 436/518; 530/323; 530/332; 530/333; 530/358

(57) ABSTRACT

The present invention relates to novel soluble combinatorial libraries, comprising a soluble phase in solution attached to a core molecule, and allowing the improved high-yield and efficient production of soluble combinatorial libraries. Some specific examples of the soluble combinatorial libraries claimed herein comprise one or more of the following: amino acids, α-azetide amino acids, triazine dione molecules, γ-lactamtide molecules, δ-lactamthiotide molecules, β-lactam nucleus containing molecules, lycoramine alkaloid nucleus containing molecules, and β-blocker nucleus molecules. Further, a split synthesis technique for generating libraries of combinatorial molecules employs a biphasic macromolecular support which is soluble during the pooling, splitting, and coupling steps but which is insoluble during the washing step. The use of a biphasic macromolecular support in its soluble phase significantly enhances the efficiency and performance of the pooling, splitting, and coupling steps. The use of a biphasic macromolecular support in its insoluble phase significantly enhances the efficiency and performance of the washing step.

### CROSS-REFERENCE TO RELATED APPLICATION

This is a continuation-in-part of pending application U.S. Ser. No. 08/281,200, filed Jul. 26, 1994, and of U.S. Ser. No. 08/484,153, filed Jun. 7, 1995.

### BACKGROUND OF THE INVENTION

This invention relates to soluble combinatorial libraries, and methods for synthesizing soluble combinatorial libraries. Such libraries are useful to drug discovery efforts and other scientific research.

The rapid production of diverse collections or libraries of chemical compounds is an important goal for those desiring to screen large numbers of novel compounds or diversomers for pharmacological activity. Combinatorial synthesis has been utilized to create libraries of molecules. These libraries often consist of oligomeric or polymeric molecules created from the sequential addition of monomeric subunits. However, typically the libraries created are not soluble and are not synthesized solution.

Dower et al., WO 91/19818 (PCT/US91/04384) describes peptide libraries expressed as fusion proteins of bacteriophage coat proteins.

Dower et al., WO 93/06121 (PCT/US92/07815) describes a method for synthesizing random oligomers and the use of identification tags to identify oligomers with desired properties.

Ellman, U.S. Pat. No. 5,288,514 describes the solid phase and combinatorial synthesis of benzodiazepine compounds on a solid support.

Huebner, U.S. Pat. No. 5,182,366 describes the controlled synthesis of peptide mixtures using mixed resins. Houghten et al., 354 Nature 84, 1991 and WO 92/09300 (PCT/US91/08694), describe the generation and use of synthetic peptide combinatorial libraries for basic research and drug discovery. These libraries are composed of mixtures of free peptides which form a heterogenous library. Systematic identification of optimal peptide ligands is achieved by screening a library followed by iterative selection and synthesis processes. For example, one library consisted of a series of six residue peptides having the first two positions specifically defined, and the last four positions consisting of a random mixture of 18 L-amino acids. This library was screened to determine which pair of defined peptides had optimum activity in an assay. A second library was then synthesized in which the optimal pair of peptides were included, the third position of each peptide individually synthesized, and the last three peptides consisted of a random mixture of 18 L-amino acids. This library was screened as before and the process repeated until the optimum six residue peptide was identified. Houghten et al. state:

“A number of other libraries, such as one composed entirely of D-amino acids, have been prepared which in total permit the systematic screening of hundreds of millions of peptides. A fundamental feature of SPCLs (synthetic peptide combinatorial libraries) is that free peptides can be generated and used in solution in virtually all existing assay systems at a concentration of each peptide most applicable to the assay. This approach has also been successfully used in radio-receptor assays (opioid peptides) and plaque inhibition assays (human immunodeficiency virus (HIV-1) and Herpes Simplex Virus (HSV)). SPCLs, as described, greatly aid all areas of drug discovery and research involving peptides.”

Lam et al., 354 Nature 82, 1991, and WO 92/00091 (PCT/US91/04666) and Houghten et al., 354 Nature 84, 1991 and WO 92/09300 (PCT/US91/08694), describe systematic synthesis and screening of peptide and other libraries of defined structure. The method used is based on a one bead one peptide approach in which a large peptide library consisting of millions of beads are screened. Each bead contains a single peptide. The authors state:

“It is clearly not enough to use a random mixture of activated amino acids in a peptide synthesis protocol, because the widely different coupling rates of different amino acids will lead to unequal representation and because each bead will contain a mixture of different peptides. Our solution was to use a ‘split synthesis’ approach. The first cycle consisted of distributing a pool of resin beads into separate reaction vessels each with a single amino acid, allowing the coupling reactions to go to completion, and then repooling the beads. The cycle was repeated several times to extend the peptide chain. In this fashion, each bead should contain only a single peptide species.”

The library of beads was screened by a staining procedure and stained beads visualized using a microscope, and removed. The structure of the peptide is obtained by a chemical analysis of the material on the single bead. Lam et al. indicate:

“Additionally, our approach has far greater potential for applying the richness of well-established peptide chemistry to synthesize libraries incorporated D-amino acids or unnatural amino acids as well as specific secondary structures including cyclic peptides. All of this can be accomplished without need to keep records of the synthetic products as our interest is focused just on those peptides which provide a strong interaction signal with the acceptor.”

These combinatorial libraries have been synthesized on a solid phase such as a bead, resin or bifer and not in solution.] The process of drug discovery has been facilitated by the advent of robotic benchmark assays which automate high-throughput screening processes. Now that new drug candidates can be screened more easily, there is an enhanced need for new drug candidates. Combinatorial chemistry, a technology which allows for the parallel synthesis of diverse molecular structures, can be employed for generating libraries of new drug candidates. (Hodgson, J., Bio/Technology 11:683-688, 1993.)

A combinatorial library is a collection of molecules having combinatorially arranged subcomponents. If all of the subcomponents belong to the same class, then the molecules which comprise the combinatorial library are polymers or oligomers. If the subcomponents belong to differing classes, then the molecules which comprise the combinatorial library or various subsets of molecules within the combinatorial library may be non-oligomeric heterocycles. Combinatorial libraries are generated by means of a split synthesis technique wherein subcomponents are added in a parallel fashion to nascent library elements contained within a combinatorial array of reaction vessels. After the addition of each subcomponent, the nascent library elements are washed, pooled, mixed, and split into a subsequent set of parallel reaction vessels for further elongation. The process of split synthesis repeats itself until a library is generated wherein each library element has the desired number of subunits. Unlike serial chemistry which produces only one species of molecule at a time, combinatorial chemistry has the potential to create molecular diversity in manner predicted according to the rules of combinometrics, exponentially, viz., Janda, K. D., Proc. Natl. Acad. Sci USA 91:10779-10785, 1994.

Combinatorial chemistry preferentially employs general reaction strategies and protocols which provide high-yielding reaction products. To this end, solid phase polymer-supported synthesis has emerged as a preferred method for the generating combinatorial libraries, e.g., Bunin, B. A., & Ellman, J. A., J. Am. Chem. Soc. 114:10997-10998, 1992; Hobbs Dewitt, S., Kiely, J. S., Stankovic, C. J., Schroeder, M. C., Reynolds, Cody, D. M. & Pavia, M. R., Proc. Natl. Acad. Sci USA 90:6909-6913, 1993; Chen. C., Ahlberg, L. A., Miller, R. B., Jones, A. D. & Kurth, M. J., J. Am. Chem. Soc. 116:2661-2662, 1994; and Backes, J. B. & Ellman, J. A., J. Am. Chem. Soc. 116:11171-11172, 1994. Solid phase polymer-supported synthesis employs an insoluble matrix substrate upon which nascent library molecules may be assembled. The insoluble matrix substrate is washed after each elongation step. The products are then pooled and split into a second or subsequent set of parallel reaction vessels for further elongation as desired.

Although the solid-phase method is a preferred method in a combinatorial chemistry, this method has certain drawbacks. The most notable liability is the heterogeneous reaction conditions, which can exhibit several of the following problems:

a) nonlinear kinetic behavior;

b) unequal distribution and/or access to the chemical reaction;

c) solvation problems;

d) the use of insoluble reagents or catalysts; and

e) pure synthetic problems associated with solid phase synthesis.

What is needed is an alternative method to solid-phase synthesis for combinatorial chemistry, i.e., a method which provides greater yields and mitigates the drawback indicated above. Outside the field of combinatorial chemistry, biphasic supports are employed in connection with the serial synthesis of individual molecules, as described below. Polyethylene glycol (PEG) is a conventional biphasic support employed in the area of serial chemistry due to its favorable physical and chemical properties. PEG polymers are available in a variety of molecular weights from 2,000 to 20,000 Dalton and can be purchased from Fluka, Sigma and Aldrich as unprotected or protected, mono or difunctionalized polymers (e.g. the monomethyl ether of PEG).

PEG products remain soluble in most reaction mixtures and organic solvents (w/v: benzene 10%, CCl₄, 10%, Dioxane 10%, Methanol, 20%, Pyridine 40%, CHCl₃ 47%, CH2Cl₂ 53%, H₂O), 55%, EtOH, 20% 34° C., EtOH 1% 32° C., EtOH0.1% 20° C., diethylether, 0.01%), but upon exposure to diethyl ether a precipitation can be effected which allows for easy separation and subsequent crystallization in cold 20° C. ethanol. Unlike other polymers, PEG avoids the tendency to form gelatinous precipitates.

PEG has been employed as a biphasic support in connection with the serial synthesis and purification of oligonucleotides, oligosaccharides and peptides. The PEG is generally connected to the core molecule through an ester linkage (e.g. a free hydroxyl on PEG is esterified via a succinate linkage to a free hydroxyl on the core molecule) however amide linkages and ether linkages are equally as accessible with PEG.

E. Bayer et al. (Nature 237:512, 1972) demonstrated the use of PEG as a biphasic support with respect to the synthesis of peptides. Bayer also disclosed that the solubilizing power of PEG was sufficient to enable the synthesis of oligomers with chain lengths up to 12 residues. In addition, Bayer noted that the physio-chemical properties of PEG-bound peptides are governed by the polymeric ester group if used as a linker (e.g., succinate linkage) up to chain lengths of 10-15 amino acid residues that are independent of the nature of the attached peptide. PEG exhibits high retention of its crystalline phase after the attachment of amorphous peptide blocks but the preparation of longer peptides (more than 20 residues) strongly depends upon the primary sequence, side-chain protection and conformation of the peptide.

Bonora et. al Nucleosides and Nucleotides 10:269, 1991, disclose a large scale dideoxynucleotide synthesis using PEG as a biphasic support and noted high yields above 90% for the synthesis of a octanucleotide with the sequence: d(TAGCGCTA). A synthesis of cyclic oligodeoxyribonucleotides, has also demonstrated the utility of the PEG support and exemplifies the precipitation and crystallization purification conditions as described herein. (Bonora et. al Nucleosides and Nucleotides, 12:21, 1993.)

Bonora discloses that biphasic supports have several comparative advantages of solid phase support systems for synthesizing these oligonucleotides, viz.:

a) the clean and convenient separation of the oligonucleotide from the reaction mixture, thereby reducing purification time;

b) the economical preparation of small oligonucleotides in milligram quantities; and

c) the advantages of the use of solution phase organic chemistry, e.g., monitoring by TLC or HPLC, variable reagents, variable temperature and pressure conditions, etc.

Krepinsky et al. disclose the preparation of milligram quantities of small PEG linked disaccharides, utilizing the crystallization purification properties of PEG. The focal point of the synthesis is that when the PEG is bound to a carbohydrate hydroxyl, the glycosylation reaction can be driven to virtual completion by repeated additions of the glycosylating agent. The excess reagents are subsequently washed off the precipitated PEG-bound product and the process is repeated until the desired length polymer is obtained. Krepinsky et al., J. Am. Chem. Soc. 113:5095, 1991.

PEG (polyethylene glycol) is a preferred biphasic support for serial syntheses due to its ease of precipitation and crystallization properties. However, alternative biphasic supports are also known in the serial chemistry area. Alternative biphasic supports include polyvinyl alcohol and polyvinylamine copolymerized with polyvinyl-pyrrolidone, etc. (Bayer et al., Nature 237:512, 1972.)

### SUMMARY OF THE INVENTION

There is provided a novel method for synthesis of soluble combinatorial libraries, allowing such synthesis to occur in a soluble phase, as opposed to on an insoluble solid phase. In this way novel libraries can be simply and readily created and assayed by more efficient means and with higher yields. Not only are the soluble combinatorial libraries of the present invention synthesized in solution, but the combinatorial libraries themselves, once synthesized, are soluble.

A combinatorial library is a collection of molecules which has or more of the following characteristics: the core molecules differ in chemical structure or composition, the assemblages of core molecules differ in chemical structure or composition, or the chemical moieties or groups of the core molecules differ in chemical structure or composition.

A soluble combinatorial library is a combinatorial library comprised of soluble molecules, and in which synthesis of the library occurs in solution and not, for example, on a solid support such as a bead or a fiber. Rather, molecules of the library are linked to a soluble, polymeric compounds.

The soluble combinatorial libraries of the present invention are particularly useful for rapidly creating and identifying large numbers of molecules that may be pharmacologically active and medicinally useful, drug candidates. The invention allows the rapid, efficient and convenient generation and screening of sets of pharmacologically active molecules. Once a pharmacologically active molecule or set of molecules has been identified, the present invention may then again be used to optimize the active molecule or set of molecules by making slight variations in the molecule or set of molecules.

An advantage of the present invention is that it allows efficient synthesis and automated screening of a large range of potentially biologically active compounds.

This invention, as will be set forth fully below, features soluble combinatorial libraries comprised of a set of related, or structurally similar, molecules wherein each molecule is attached to a soluble polymeric molecule. The invention includes efficient methods of synthesizing soluble combinatorial libraries, and compositions for generating soluble combinatorial libraries. The invention allows for great flexibility in the format of synthesis.

In one embodiment, the invention is a soluble combinatorial library that allows improved manipulation of library molecules, generation of larger libraries and improved and efficient purification of compositions comprising the library. Further, the combinatorial libraries themselves are soluble in solution, an advantage over conventional solid phase synthesis.

In the preferred embodiment, the soluble combinatorial libraries are comprised of “core molecules” or an “assemblage of core molecules”. The core molecules are compounds that share a common chemical, structural or functional element, and determine the identity of the soluble combinatorial library. The core molecules or assemblage of molecules may differ, however, in one or more chemical moieties.

A “set of core molecules” is two or more core molecules which differ in their respective chemical moieties. An “assemblage of core molecules” is a series of two or more core molecules chemically linked together.

Examples of core molecules, which are not meant to limit the scope of the present invention are: amino acids, α-azetide amino acids, triazine dione molecules, γ-lactamtide molecules, δ-lactamthiotide molecules, β-lactam nucleus containing molecules, lycoramine alkaloid nucleus containing molecules, and β-blocker nucleus molecules or combinations thereof.

A “soluble polymeric compound” is a molecule that may be dissolved in the solvents in which the desired library synthesizing reactions are to occur. Related compounds that may be insoluble alone may become soluble in a desired solvent once the related compound is attached to the soluble polymeric compound. In this way, the reagents can be reacted in solution with a core molecule. Typically, such reactions in solution are inefficient or impossible when the reagents are reacted with a core molecule that is not attached to a soluble polymeric compound.

The present invention, by synthesizing on a soluble polymeric compound, creates yields that are several orders of magnitude larger than those produced by solid phase synthesis. Typically, the present invention yields milligrams and grams of product, and the present invention's yields may be unlimited. In contrast, solid phase synthesis yields only scant nanograms of product.

The soluble polymeric compound used in synthesizing the soluble combinatorial libraries of the present invention may be inert to the chemicals that are to be reacted with the core molecules, but may be otherwise chemically active. The core molecules can be treated with reagents without fear of unwanted side reactions between the reagents and the soluble polymeric compound. Still, the soluble polymeric compound may react with certain other chemicals that will not react with the core molecules or products therefrom.

A further advantage of the soluble polymeric compound is that it facilitates efficient isolation and recovery of the desired product.

The soluble polymeric compound may be of a size and weight that allows it to be readily isolated through filtration and chromatographic techniques that are well known to those skilled in the art.

Also, the soluble polymeric compound may be cleavable from the core molecules. Through the use of reagents and chemicals that react with the soluble polymeric compound, but that do not react with the core molecules, the soluble polymeric compound may be chemically modified so that it becomes insoluble, facilitating purification and isolation of the attached core molecules.

The present invention may be utilized to create a soluble combinatorial library that may later be rendered insoluble. The present invention, therefore, has all of the advantages of conventional syntheses and conventional libraries, such as those produced by solid phase synthesis—insoluble libraries—and has many additional advantages not exhibited by conventional syntheses and libraries. A few of these additional advantages are rapid and efficient production, high yields, and the production of soluble combinatorial libraries.

The soluble polymeric compound may be reacted with other chemicals so that an attached core molecule may be cleaved from the soluble polymeric compound and recovered.

A “composition” is one of a set of core molecules that may have been chemically modified to add or remove one or more chemical moieties.

The libraries can be comprised of any combination of compounds containing different moieties. These compounds may be synthesized by traditional chemical methods or through the use of enzymes. The moieties may be natural or unnatural, and may include amino acids, the R groups of amino acids such as the hydroxyl group in methionine, nucleotides or portions thereof, sugars, lipids and carbohydrates. The bond used to attach each group can be of any type, including covalent ionic and coordination bonds. The bonds may be cleaved selectively by enzyme or chemical treatment.

The present invention is useful because it allows the quick and efficient generation of a diverse set of core molecules that can be isolated and purified by several efficient methods due to the attachment of a soluble compound. The compound can remain attached or be cleaved as desired at any stage of the purification. For example, use of PEG as the soluble compound allows rapid and efficient isolation by membrane filtration or precipitation.

The present invention is also useful for the generation of larger libraries than those libraries that can be created by traditional solid phase synthesis.

The present invention is also useful for screening for novel therapeutic molecules. For example, the invention allows one to screen for such therapeutic molecules as receptor agonists or antagonists.

Also, if α-aza amino acids are used as a backbone, the possibility of oral administration of the library molecules is created. This possibility is due to the fact that the α-aza amino acid are not easily hydrolyzed. Further, in α-aza amino acids the α-carbon of the traditional amino acids is replaced by a nitrogen. This nitrogen potentially allows for enhanced pharmacological activity in the library molecules that contain the α-azetide.

In other aspects, the invention features the following compositions which may be coupled by the soluble-reaction method of the present invention or any other combinatorial library generation method as would be known to one of ordinary skill in the art: α-azetide compositions; triazine dione compositions, which are nucleic acid-like compounds; γ-lactamtides and δ-lactamthiotides, the latter of which can be derived from a protected cysteine; a β-lactam nucleus, wherein the chemical groups may be varied; a lycoramine alkaloid nucleus, wherein the chemical moieties or groups attached thereto may be varied; and a β-blocker nucleus comprising, for example, a naphthol or phenol ring wherein the chemical moieties or groups about the ring may be varied.

Further provided are the following combinatorial libraries: a polyoxygenated compound library, comprising heterocyclic compounds containing two or more oxygen atoms and at least one chemical moiety or group, wherein the compounds may be coupled along a backbone that is peptide or peptide-like; an aryloxyacetic acid library; a polyether backbone compound library; a pyridyl backbone compound library; and a dideoxynucleotide compound library.

In a preferred embodiment, synthesis of the soluble combinatorial library utilizes polyethylene glycol (“PEG”) as the soluble polymeric compound to which an initial core molecule of the combinatorial library is attached. The soluble, polymeric protecting group may include, for example, polyvinyl alcohol or polyvinylamine copolymerized with polyvinylpyrrolidone. A number of core molecules are typically utilized in generating the combinatorial library, typically one core molecule per reaction vessel is used.

Optionally, a precipitation step may then be performed in each reaction vessel. The precipitation step allows for the purification of the PEG-molecules from core molecules which have not coupled to PEG, as only PEG-molecules precipitate and may be separated from molecules remaining in solution. After the purification step a mixing step may be performed. The mixed PEG-molecules may then be reacted with another core molecule. This process is continued until the desired number of core molecules have been coupled. Finally, the PEG is cleaved leaving the coupled core molecules as the desired products.

In another embodiment, the shared structural elements are one or more peptide bonds, and the chemical moieties are polyoxygenated heterocyclic compounds.

In another embodiment, the shared structural element is a β-lactam, and the chemical moieties may be attached to either the carboxy or amino functional groups of the β-lactam. Moieties that may be attached to the carboxy functional group include, but are not limited to, alcohols, amino acids and amines. Moieties that may be attached to the amino group include, but are not limited to, esters, amino acids, carbamates and thioesters.

Other and further objects, features and advantages will be apparent from the following description of the presently preferred embodiments of the invention.

The invention is directed to an improved method for generating a library of combinatorial molecules. The method employs at least two cycles of a parallel split synthesis. The cycle of the parallel split synthesis commences by collecting and mixing biphasic macromolecular supports within a common pool. Each macromolecular support has a nascent library molecule attached to it. The common pool of biphasic macromolecular supports is then split and transferred to a series of separate reaction vessels. There, the nascent library molecules are elongated within each separate reaction vessel by the parallel addition of reactants to reaction vessel within a first solvent which renders the biphasic macromolecular supports soluble, e.g., alcohol. Then, the biphasic macromolecular supports are rendered insoluble by addition of a second solvent and washed for removing reactants therefrom. The cycle may then be repeated as desired for generating the library of combinatorial molecules.

In a preferred mode, the biphasic macromolecular supports are selected from the group consisting of polyethylene glycol (PEG), polyvinylalcohol, polyvinylamine copolymerized with polyvinyl pyrrolidine, and derivatives thereof. These biphasic macromolecular supports are rendered soluble by the addition of an alcohol and may be rendered insoluble by addition of an ether. Preferred combinatorial molecules include oligopeptides, oligosaccharides, oligonucleotides, arylsulfonamides, and derivatives thereof.

In a preferred alternative mode, a deconvolution assemblage is synthesized in parallel with the library of combinatorial molecules. The deconvolution assemblage is employable for deconvoluting the identity of combinatorial molecules identified as positive by an assay. The deconvolution assemblage is formed by removing an aliquot of the macromolecular supports from each reaction vessel after the washing step.

The parallel split synthesis using a biphasic macromolecular support has the advantages of a stepwise synthesis without cumbersome intermediate purification procedures. Amino acids, nucleotides, arylsulfonamides or sugar residues, are shown to be reversibly, covalently linked to a biphasic macromolecular support which determines the physical and chemical properties of the growing oligomeric chain during all stages of the synthesis. All reactions utilize standard solution phase organic chemistry. This provides flexibility to use variable quantities of reagents, if necessary, e.g. stoichiometric or large excess, and permits increased reaction times and variable temperatures to drive the reaction to completion. In addition, the reactions can be monitored by standard TLC or HPLC analysis. A further benefit is that the polymer-bound core molecule can be purified from tahe soluble reagents by simple precipitation and/or crystallization procedures which are reflective of the biphasic macromolecular support. The biphasic macromolecular support also has the advantage of differing in molecular size with the reagents, such that a simple filtration via dialysis by membrane filtration etc., may be all that is necessary to purify the product after the coupling steps. (E. Bayer et al., Nature 237:512, 1972.)

Use of a biphasic macromolecular support imparts the following advantages:

1) The synthesis cycle is simplified, purification methods utilize filtration, precipitation and/or crystallization methodologies which reflect the physical properties characteristic of the biphasic macromolecular support. Hence the total time required for the synthesis of an oligomer is shortened.

2) Solubilities of the core molecules may be enhanced (e.g., peptides) via the covalent linkage between the core molecules and the biphasic macromolecular support (e.g., polymeric ester groups).

3) The use of filtration, precipitation or crystallization properties with respect to the biphasic macromolecular support permits the use of less expensive methods and concomitantly allows the rapid production of high yielding products.

Use of a biphasic macromolecular support achieves, in essence, a parallel split synthesis for generating libraries of combinatorial molecules in which the pooling, splitting, and elongation steps occur in liquid phase while the wash step is performed on a solid phase. By adopting this approach, the drawbacks of a wholly solid-phase combinatorial synthesis are avoided, while the positive aspects are preserved. Conversely, the advantages of a liquid phase synthesis is achieved without the disadvantages thereof. This strategy can be termed Liquid Phase Combinatorial Synthesis (LPCS).

In a preferred embodiment of LPCS, linear homopolymer (polyethylene glycol monomethylether (MeO-PEG)) is employed as the biphasic macromolecular support. The MeO-PEG also serves as a terminal protecting group for the library of compounds synthesized. This mono-functional polymer was selected as the homopolymer “protecting group” of choice, because of its successful application in peptide, oligonucleotide/oligosaccharide synthesis. (Bayer, E. & Mutter, M., Nature 237:512-513, 1972; Bonora, G. M., Scremin, C. L., Colonna, F. P. & Garbesi, A., Nucleic Acid Res. 18:3155-3159, 1990; and Douglas, S. P., Whitfield, D. M. & Krepinsky, J. J., J. Am. Chem. Soc. 113:5095-5097, 1991.)

Two properties that are inherent in this homopolymer's structural makeup provide the necessary elements for it to be attractive in a combinatorial format. First, due to its helical structure MeO-PEG has a strong propensity to crystallize (Rajasekharan Pillai, V. N. & Mutter, M;, Acc. Chem. Res. 14:122-130, 1981.) Thus as long as the polymer remains unaltered during the construction of the library, purification by crystallization can be accomplished at each stage of the combinatorial process. Second, MeO-PEG has remarkable solubilizing effects in a variety of aqueous and organic solvents (Bayer, E., Mutter, M., Poster, J. & Uhmann, R., Pept. Proc. Eur. pept. Sympo. 13:129-136, 1975.) This solubilizing feature, found in the “liquid phase” process, can be used in an advantageous manner if the homopolymer is treated as a “reagent” and used in large excess. Under such conditions, a quantitative reaction may be achieved. In contrast, classical solid-phase synthesis can not provide the combinatorial user with this type of alternative chemistry. Another added virtue of MeO-PEG's favorable solubility properties is that all manipulations in the LPCS methodology, including split synthesis, may be carried out under homogeneous conditions. Furthermore, because LPCS is a solution phase process our “recursive deconvolution strategy” can be used to create and screen the library of interest (Erb, E. Janda, K. D. & Brenner, S., Proc. Natl. Acad. Sci USA 91:11422-11426, 1994.) Lastly, yields from the individual combinatorial reaction steps can be monitored by either Carbon-13 or Proton-Nuclear Magnetic Resonance spectroscopy.

### BRIEF DESCRIPTION OF THE DRAWINGS

First, a brief description of the figures follows:

FIG. 1 is an illustration of the synthesis of a soluble combinatorial library of β-blocker compositions.

FIG. 2 is an illustration of the synthesis of a soluble combinatorial library of lactamtide compositions.

FIG. 3 is an illustration of the synthesis of a soluble combinatorial library of lactamthiotide compositions.

FIG. 4 is an illustration of the synthesis of a soluble combinatorial library of γ-lactampeptide compositions.

FIG. 5 is an illustration of the synthesis of a soluble combinatorial library of aryloxyacetic acid compositions.

FIG. 6 is an illustration of the synthesis of a soluble combinatorial library of polyether backbone compositions.

FIG. 7 is an illustration of the synthesis of a soluble combinatorial library of highly oxygenated amino acid compositions.

FIG. 8 is an illustration of the synthesis of a soluble combinatorial library of highly oxygenated amino acid compositions.

FIG. 9 is an illustration of the synthesis of a soluble combinatorial library of highly oxygenated compositions.

FIG. 10 is an illustration of the synthesis of a soluble combinatorial library of triazinedione compositions.

FIG. 11 is an illustration of the synthesis of a soluble combinatorial library of nucleoside analog compositions.

FIG. 12 is an illustration of the synthesis of a soluble combinatorial library of lycoramine compositions.

FIG. 13 is an illustration of the synthesis of a soluble combinatorial library of β-lactam compositions.

FIG. 14 is an illustration of the synthesis of a soluble combinatorial library of aza-amino-acid compositions.

FIG. 15 is an illustration of the synthesis of a soluble combinatorial library of azapeptide compositions.

FIG. 16 is a schematic of a combinatorial library synthesis using recursive deconvolution on a soluble support.

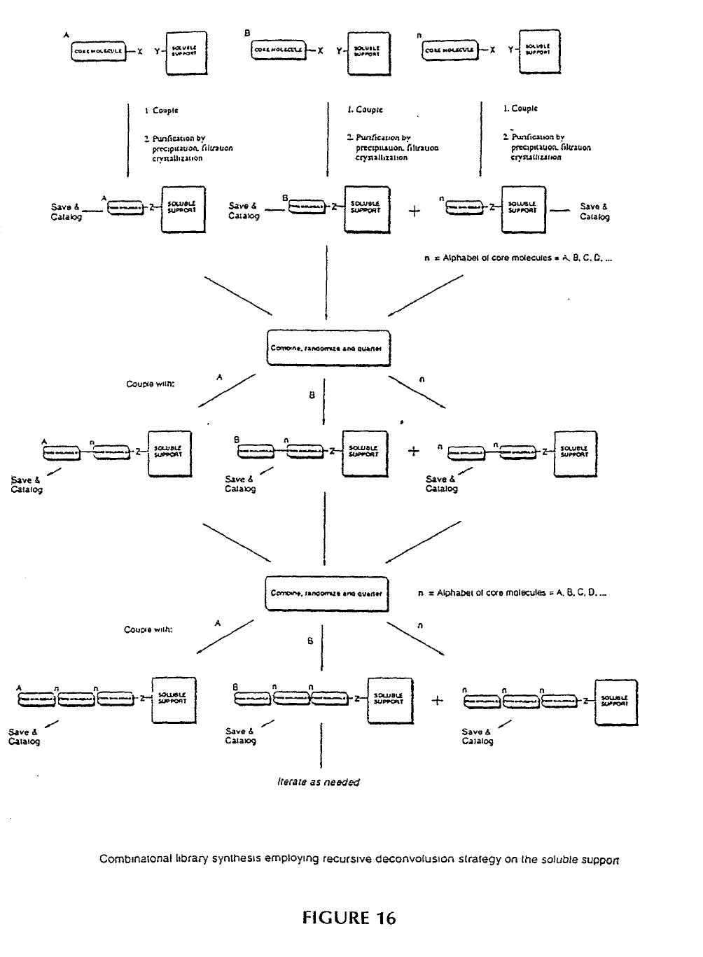

FIG. 17 illustrates synthesis of core molecules 11 and 12.

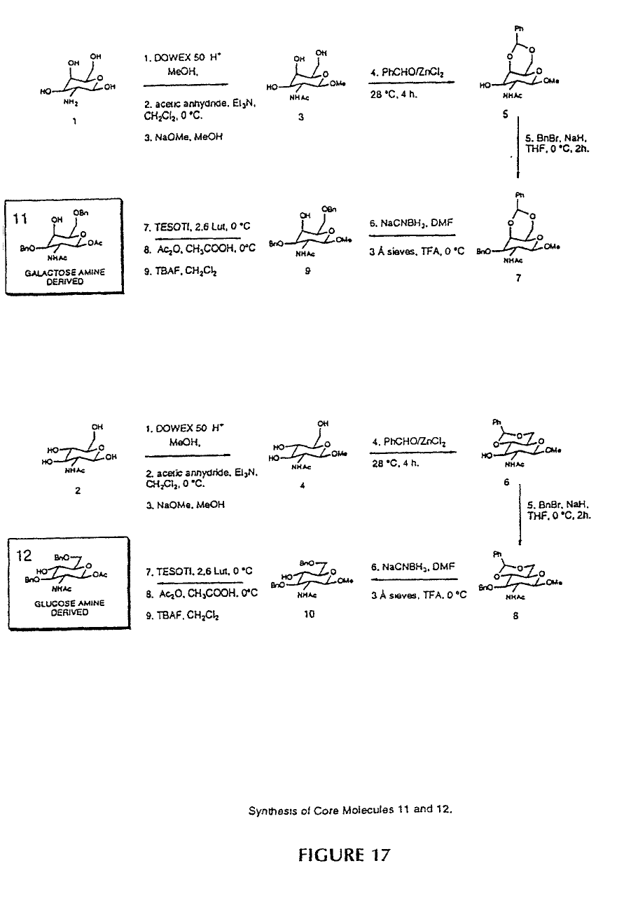

FIG. 18 illustrates synthesis of core molecules 23 and 24.

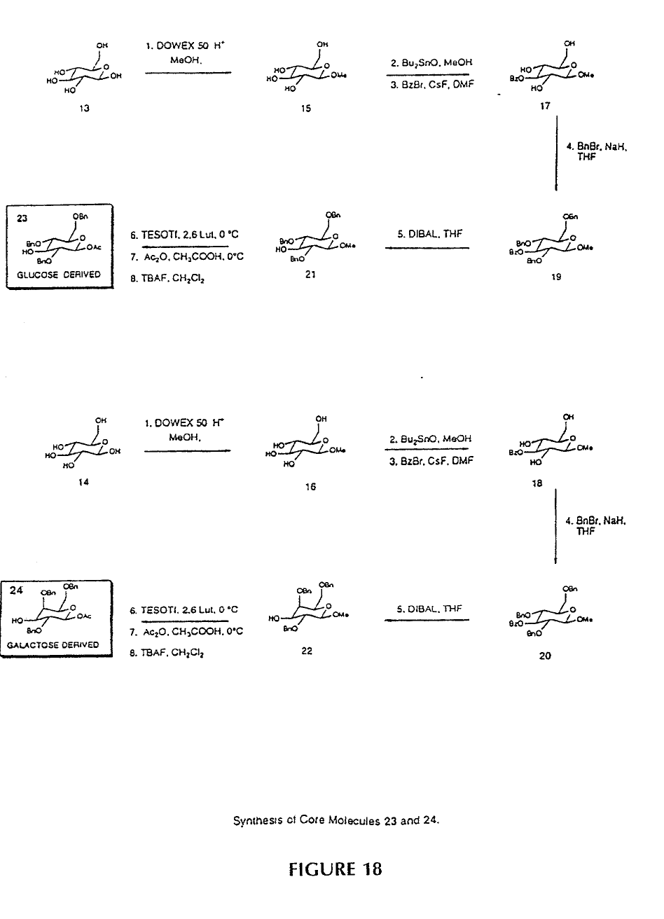

FIG. 19 illustrates attachment of PEG support to core molecules.

FIG. 20 illustrates synthesis of library 2.

FIG. 21 illustrates synthesis of library 3.

FIG. 22 illustrates synthesis of library 4.

FIG. 23 illustrates synthesis of library 5.

FIG. 24 illustrates final purification of libraries.

FIG. 25 illustrates nucleotide split synthesis with a PEG support.

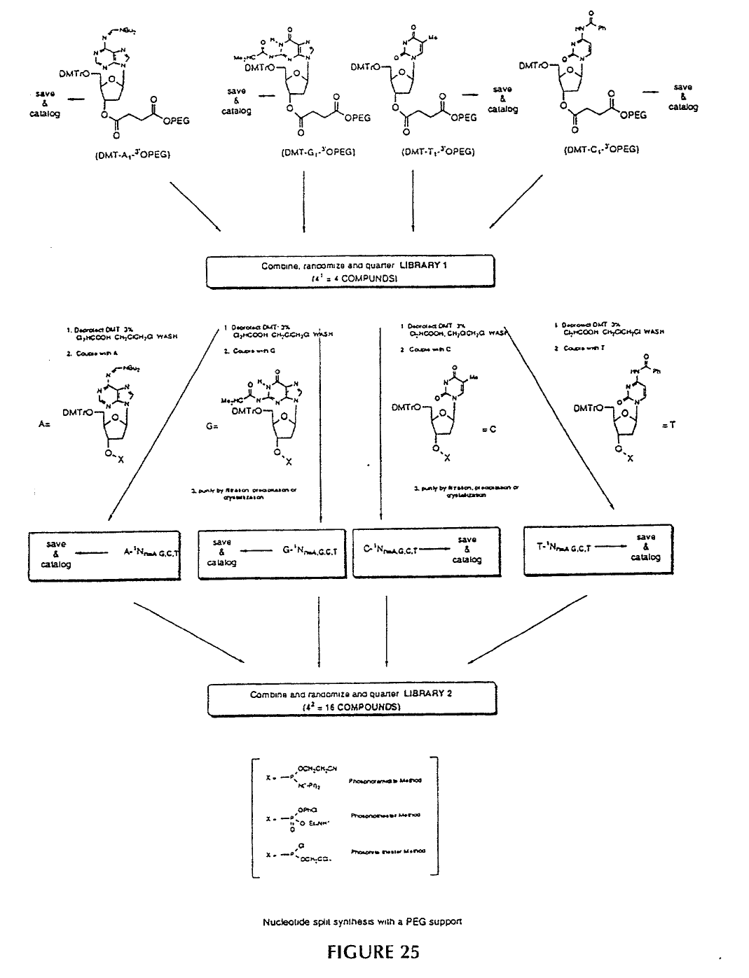

FIG. 26 illustrates oligonucleotide split synthesis with PEG support.

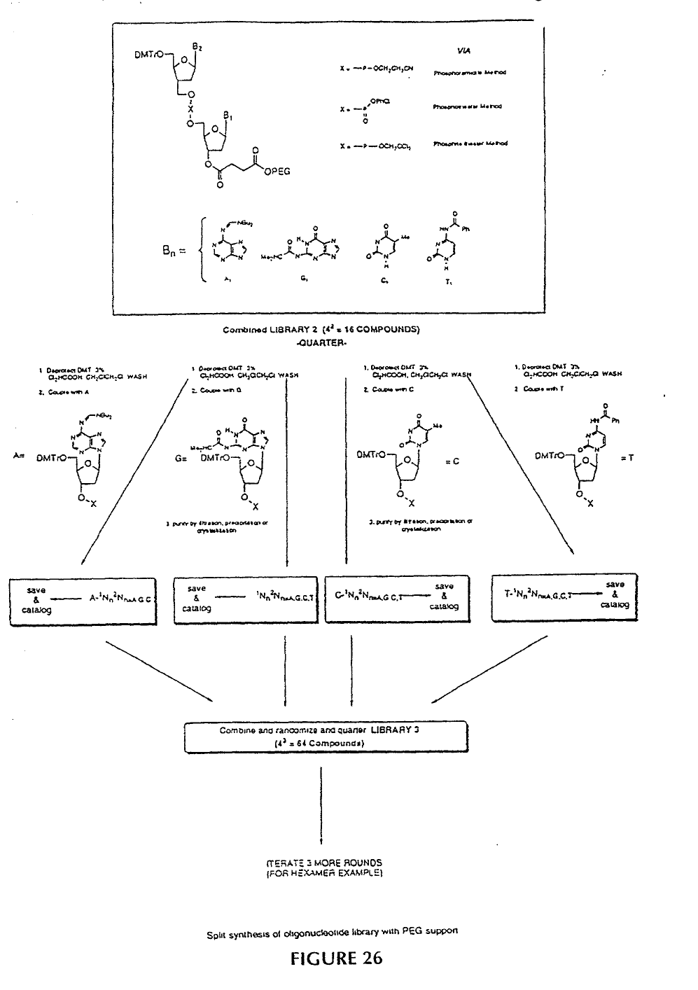

FIG. 27 illustrates a hexamer after 5 rounds of coupling.

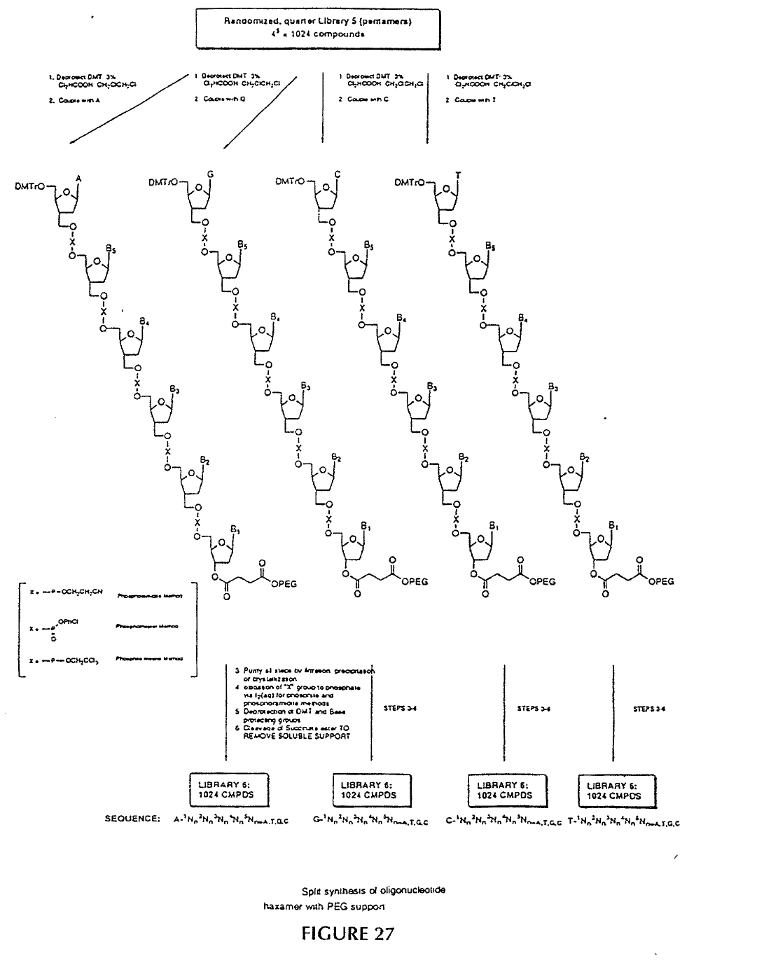

FIG. 28 illustrates preparation [Leu⁵]-=enkephaline-bovine serum albumin conjugate.

FIG. 29 illustrates two arylsulfonamide preparatio methods.

FIG. 30 illustrates construction of an arylsulfonamide library.

FIG. 31 illustrates recursive deconvolution of peptide library containing the antigenic determinant Tyg-Gly-Gly-Phe-Leu recognized by monoclonal antibody 3E7.

FIG. 32 illustrates arylsulfonamide derivatives 7.

FIG. 33 illustrates the structure of compounds 3 through 8.

FIG. 34 illustrates the structure of compounds 4 through 18.

FIG. 35 illustrates the structure of compounds 19 through 24.

FIG. 36 illustrates the structure of compounds 501 through 503.

FIG. 37 illustrates the structure of compounds 504 through 506.

FIG. 38 illustrates the structure of compounds 507 through 508.

FIG. 39 illustrates the structure of compound 509 through 511.

FIG. 40 illustrates the structure of compound 512 through 514.

FIG. 41 illustrates a one-pot synthesis of azadipeptides.

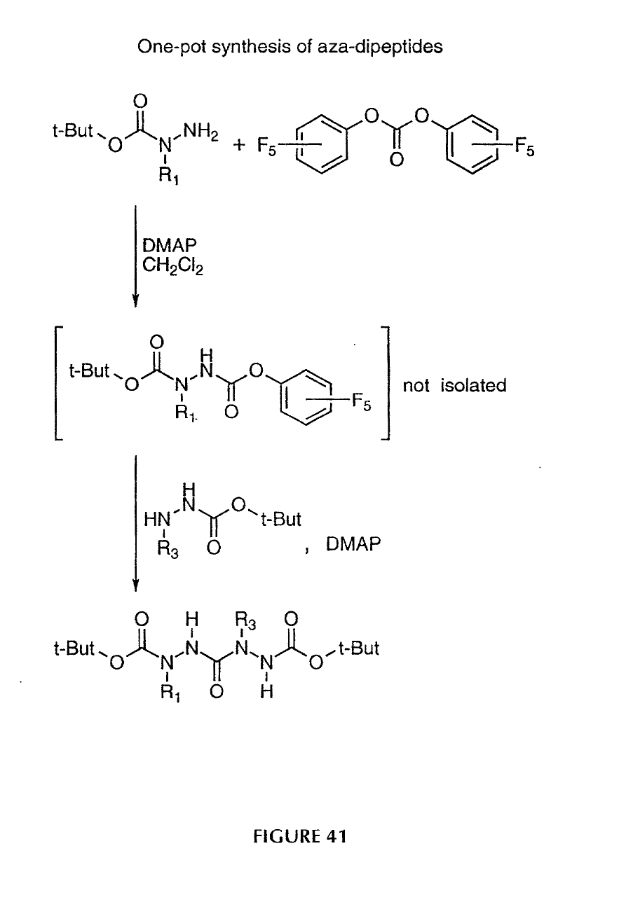

FIG. 42 illustrates yields of various azadipeptides.

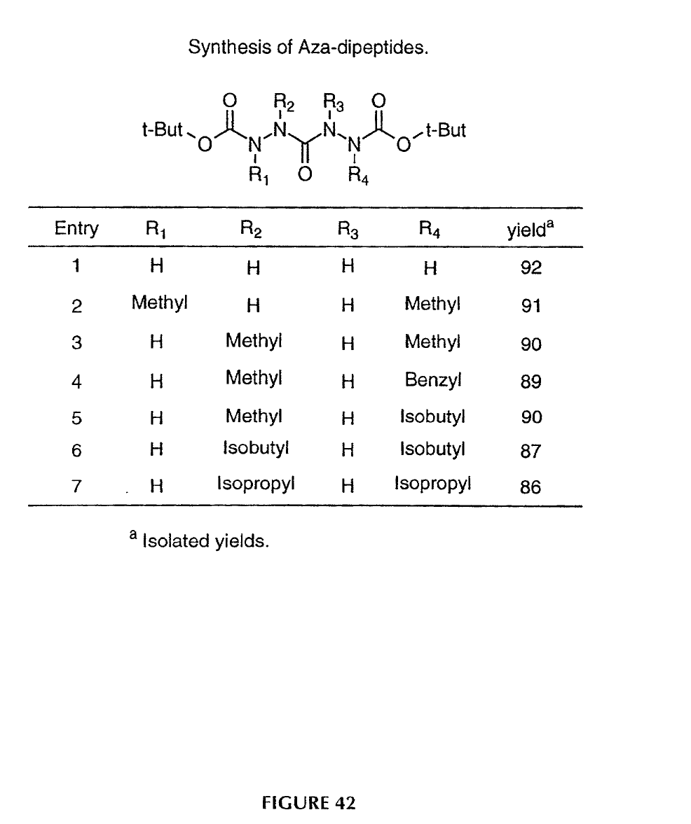

FIG. 43 illustrates a scheme for MeO-PEG-supported aza-peptide synthesis.

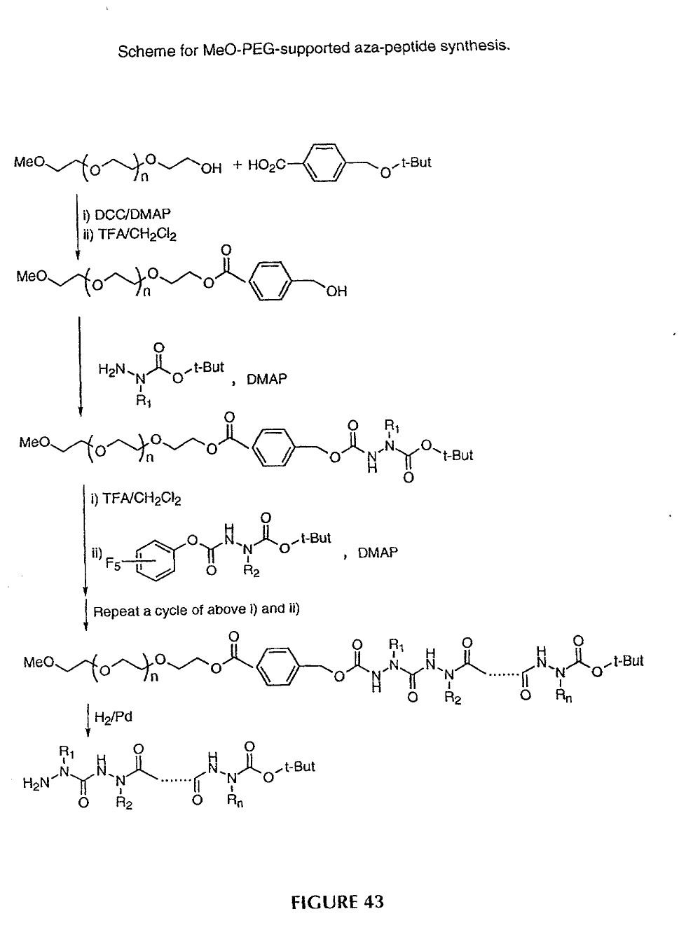

### DETAILED DESCRIPTION OF THE INVENTION

A soluble combinatorial library is provided. Such a combinatorial library allows easier, improved manipulation of core molecules, permits generation of larger libraries and allows easier, more efficient purification of compositions comprising the library. Moreover, the combinatorial library itself, once synthesized on the soluble polymeric compound, is soluble.

This invention relates to synthesis of combinatorial libraries. A key aspect of the present invention is that it allows the rapid, efficient creation of a large diverse library of molecules having core molecules that may differ in molecular structure, size or both.

A generalized protocol for the parallel split synthesis for concurrently generating a library of combinatorial molecules and a deconvolution assemblage is illustrated in Scheme 1. A soluble support, i.e., a biphasic macromolecular support, is aliquoted or split into a series of n parallel reaction vessels. To each of the series of reaction vessels is added a corresponding species of a series of n core molecules, e.g., a first core molecule may be added to the first reaction vessel, a second core molecule may be added to the second reaction vessel, etc. The core molecule is then allowed to couple to the soluble support (or biphasic macromolecular support) to form a nascent combinatorial molecule or to elongate a nascent combinatorial molecule. The biphasic macromolecular support is then crystallized or otherwise converted to a solid phase and then washed. Each of the washed products is then resolubilized on its biphasic macromolecular support and an aliquot is taken and saved to form a deconvolution assembly. The remainder is added to a common pool. The common pool is mixed and split to a second series of reaction vessels. The cycle of elongation, washing, and splitting may be repeated a second time. The cycle may be repeated as required until an entire library of combinatorial molecules is generated.

The practical utility of the present invention is as follows: The invention is useful for, among other things, developing new drugs. The invention is also useful for rapidly generating and developing large numbers of drug candidate molecules. The invention is useful for systematically synthesizing a large number of molecules that may vary greatly in their chemical structure or composition, or that may vary in minor aspects of their chemical structure or composition. The invention is also useful for randomly generating a large number of drug candidates, and later optimizing those candidates that show the most medicinal promise.

The methods used to generate combinatorial libraries, for example the split synthesis method, are also compatible with the combinatorial libraries of this invention. Split synthesis is carried out as follows: The first step entails adding ten different molecules A, B, C . . . J, to ten separate vessels. The contents of these vessels are mixed or pooled, divided into ten new different vessels, and ten further parallel syntheses are carried out to provide the core molecules XA1, XB1, XC1 . . . XJ1, where X is any one of the original A-J, and A1, B1, C1 . . . J1 are ten different molecules which may be the same or different from A-J. Of course, fewer or more than ten syntheses can be used in this second step. In the third step, the contents of the vessels are again mixed and divided into ten further vessels so that the synthetic procedure can be repeated until the entirety of each desired core molecule is synthesized.

The final ten vessels in the above example (each having a diversity of core molecules, core molecule chemical groups and/or core molecule assemblages with a known subunit at their terminus) can be assayed using any standard assay format. That is, each of the ten mixtures is assayed to determine which mixture contains one or more active compounds.

Such a combinatorial library may contain tens, thousands, or more core molecules. The number of core molecules that can be generated by the present invention, is, in fact, unlimited. For example, a large library of molecules that could possibly bind to a certain enzyme receptor site can be rapidly generated and screened. The molecule that binds to the enzyme or receptor site can then be quickly and accurately assayed and administered using the screening and therapeutic methods detailed below.

Synthesis of the combinatorial library occurs on a soluble polymeric molecule in solution. By synthesizing the combinatorial library on a soluble polymeric molecule in solution, the present invention permits library synthesis that is more rapid and efficient than conventional methods, such as solid phase synthesis. The present invention creates a soluble combinatorial library wherein the synthesis steps may occur in solution. By synthesizing in solution, the present invention produces yields that are several orders of magnitude greater than, say, solid phase synthesis yields. Solid phase synthesis typically yields only a few nanograms of product. In contrast, the present invention, by synthesizing in solution, yields milligrams and grams of product.

The soluble polymeric molecules are attached to core molecules. Core molecules share one or more common chemical, structural or functional elements. Core molecules that may be insoluble in desired reaction solvents may become soluble once attached to the soluble polymeric molecule. The acquired solubility of the core molecules allows efficient chemical manipulation of the core molecules in solution.

Core molecules may also contain chemical moieties. A core molecule, once in solution by virtue of being bonded to a soluble polymeric molecule, may then be chemically altered by chemically modifying the molecule's moiety. In this way a large set of diverse core molecules can be created. A given set of core molecules may be diversified, also, by the substitution of the core molecules' moieties.

A clear advantage of the present invention is that it allows the rapid generation of a large, highly diversified library of molecules. The high diversity of the library allows rapid and accurate experiments and assays to be performed on a large diversity of core molecules.

The invention is particularly useful for the creation and isolation of a large number of core molecules which may be screened for pharmacological activity. A large set of molecules could then further screened for therapeutic uses. Once a promising target molecule is identified, the present invention can be used to optimize the molecule by rapidly and efficiently synthesizing and assaying a large diverse library of molecules that vary the target molecule, by the addition, removal or alteration of, for example, a functional group.

The present invention also allows for aliquot sampling of the reaction mixture at each step in the synthesis of a combinatorial library. The structure of the molecules present in each aliquot is recorded. Once a pharmacologically active “target” molecule is identified, the target molecule's exact structure and composition can then be accurately and quickly ascertained, as is known in the art.

Another advantage of the present invention is that the invention can be executed in a variety of formats, including manual and automated formats.

Molecules in the soluble combinatorial library of the present invention may also be purified by any of the techniques well known in the art. These techniques include, but are not limited to, precipitation, thin layer chromatography, column chromatography, high pressure liquid chromatography, crystallization, gel electrophoresis, and filtration.

PEG is a preferred soluble polymeric compound for the soluble combinatorial libraries of the present invention. However, other compounds, including, polyvinyl alcohol and polyvinylamine copolymerized with polyvinylpyrrolidone may be utilized.

A preferred set of core molecules comprises the class of lactamtide molecules. Another preferred set of core molecules comprises the class of dideoxynucleotides, including naturally occurring and unnatural nucleotides. Another preferred set of core molecules comprises aryloxyacetic compositions. Another preferred set of core molecules comprises polyether compositions. Another preferred set of core molecules comprises the class of polyoxygenated amino acids. Another preferred set of core molecules comprises amino acid compositions. Another preferred set of core molecules comprises triazine-dione compositions. Another preferred set of core molecules comprises the class of β-blocker molecules. Another preferred set of core molecules comprises lycoramine-nucleus compositions. Another preferred set of core molecules comprises β-lactam compositions. Another preferred set of core molecules comprises pyridyl compositions. Another preferred set of core molecules comprises α-azetide compositions.

Screening of a Soluble Combinatorial Library

A soluble combinatorial library of the present invention may be screened by any method well known in the art. These methods include, but are not limited to, ELIZA plating, receptor binding, southern, western and northern blotting, and competitive binding. One such method for identifying an agent to be tested for an ability to bind to and potentially modulate a cellular receptor signal transduction pathway is as follows. The method involves exposing at least one compound from the combinatorial libraries of the present invention to a protein comprising a functional portion of a cellular receptor for a time sufficient to allow binding of the combinatorial library compound to the functional portion of the cellular receptor; removing non-bound compound; and determining the presence of the compound bound to the functional portion of the cellular receptor, thereby identifying a compound to be tested for an ability to modulate a cellular receptor signal transduction pathway.

One method utilizing this approach that may be pursued in the isolation of such receptor-binding molecules would include the attachment of a combinatorial library molecule, or a portion thereof, to a solid matrix, such as agarose or plastic beads, microtiter wells, petri dishes, or membranes composed of, for example, nylon or nitrocellulose, and the subsequent incubation of the attached combinatorial library molecule in the presence of a potential combinatorial library molecule-binding compound or compounds. Attachment to said solid support may be direct or by means of a combinatorial-library-compound-specific antibody bound directly to the solid support. After incubation, unbound compounds are washed away, component-bound compounds are recovered. By utilizing this procedure, large numbers of types of molecules may be simultaneously screened for receptor-binding activity.

Administration of the Featured Compounds

After a promising compound has been identified by a screening method, the identified compound can be administered to a patient alone, or in a pharmaceutical composition comprising the identified active compound and a carrier or excipient. The compounds can be prepared as pharmaceutically acceptable salts (i.e., non-toxic salts which do not prevent the compound from exerting its effect).

Pharmaceutical compositions suitable for use in the present invention include compositions wherein the active ingredients are contained in an effective amount to achieve its intended purpose. Determination of the effective amounts is within the capability of those skilled in the art, especially in light of the detailed disclosure provided herein. The pharmaceutical compositions of the present invention may be manufactured in a manner that is itself known, e.g., by means of conventional mixing, dissolving, granulating, dragee-making, levigating, emulsifying, encapsulating, entrapping or lyophilizing processes.

Pharmaceutical preparations for oral use can be obtained, for example by combining the active compounds with solid excipient, optionally grinding a resulting mixture, and processing the mixture of granules, after adding suitable auxiliaries, if desired, to obtain tablets or dragee cores. Suitable excipients are, in particular, fillers such as sugars, including lactose, sucrose, mannitol, or sorbitol; cellulose preparations such as, for example, maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP). If desired, disintegrating agents may be added, such as the crosslinked polyvinyl pyrrolidone, agar, or alginic acid or a salt thereof such as sodium alginate.

Pharmaceutically acceptable salts can be prepared by standard techniques. For example, the free base form of the compound is first dissolved in a suitable solvent such as an aqueous or aqueous-alcohol solution, containing the appropriate acid. The salt is then isolated by evaporating the solution. In another example, the salt is prepared by reacting the free base and acid in an organic solvent.

Carriers or excipient can be used to facilitate administration of the compound, for example, to increase the solubility of the compound. Examples of carriers and excipients include calcium carbonate, calcium phosphate, various sugars or types of starch, cellulose derivatives, gelatin, vegetable oils, polyethylene glycols and physiologically compatible solvents. The compounds or pharmaceutical composition can be administered by different routes including intravenously, intraperitoneally, subcutaneously, and intramuscularly; orally, topically, or transmucosally.

For injection, the agents of the invention may be formulated in aqueous solutions, preferably in physiologically compatible buffers, such as physiological saline buffer. For such transmucosal administration, penetrants appropriate to the barrier to be permeated are used in the formulation. Such penetrants are generally known in the art.

Use of pharmaceutically acceptable carriers to formulate the compounds herein disclosed for the practice of the invention into dosages suitable for systemic administration is within the scope of the invention. With proper choice of carrier and suitable manufacturing practice, the compositions of the present invention, in particular, those formulated as solutions, may be administered parenterally, such as by intravenous injection. The compounds can be formulated readily using pharmaceutically acceptable carriers well known in the art into dosages suitable for oral administration. Such carriers enable the compounds of the invention to be formulated as tablets, pills, capsules, liquids, gels, syrups, slurries, suspensions and the like, for oral ingestion by a patient to be treated.

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Agents intended to be administered intracellularly may be administered using techniques well known to those of ordinary skill in the art. For example, such agents may be encapsulated into liposomes, then administered as described above. Liposomes are spherical lipid bilayers with aqueous interiors. All molecules present in an aqueous solution at the time of liposome formation are incorporated into the aqueous interior. The liposomal contents are both protected from the external microenvironment and, because liposomes fuse with cell membranes, are efficiently delivered into the cell cytoplasm.

All patents and publications mentioned in this specification are indicative of the levels of those skilled in the art to which the invention pertains. All patents and publications are herein incorporated by reference to the same extent as if each individual publication is specifically and individually indicated to be incorporated by reference.

It will be readily apparent to one skilled in the art that various substitutions and modifications may be made to the invention disclosed herein without departing from the scope and spirit of the invention.

Chemical Definitions

The following is a list of some of the definitions used in the present disclosure.

An “alkyl” group refers to a saturated aliphatic hydrocarbon, including straight-chain, branched-chain, and cyclic alkyl groups. The alkyl group may have 1 to 12 carbons, or may have 3 to 9 carbons. The alkyl group may be substituted or unsubstituted. When substituted the substituted groups may be hydroxyl, cyano, alkoxy, ═O, ═S, NO2 or N(CH3)2, amino, SH, or aryl.

An “alkenyl” group refers to an unsaturated hydro-carbon group containing at least one carbon-carbon double bond, including straight-chain, branched-chain, and cyclic groups. The alkenyl group may have 1 to 12 carbons, or may have 3 to 9 carbons. The alkenyl group may be substituted or unsubstituted. When substituted the substituted groups may be hydroxyl, cyano, alkoxy, ═O, ═S, NO2 or N(CH3)2, amino, SH, or aryl.

“alkynyl” group refers to an unsaturated hydro-carbon group containing at least one carbon-carbon triple bond, including straight-chain, branched-chain, and cyclic groups. The alkynyl group may have 1 to 12 carbons, or may have 3 to 9 carbons. The alkynyl group may be substituted or unsubstituted. When substituted the substituted groups may be, hydroxyl, cyano, alkoxy, ═O, ═S, NO2 or N(CH3)2, amino, SH, or aryl.

An “alkoxyl” group refers to an “—O-alkyl” group, where “alkyl” is defined as described above.

An “aryl” group refers to an aromatic group which has at least one ring having conjugated pi electron system and includes carbocyclic aryl, heterocyclic aryl and biaryl groups, all of which may be optionally substituted. The substituents of the aryl groups may be hydroxyl, cyano, alkoxy, alkyl, alkenyl, alkynyl, amino, or aryl groups.

An alkylaryl group refers to an alkyl (as described above) covalently bonded to an aryl group (as described above).

Carbocyclic aryl groups are groups wherein the ring atoms on the aromatic ring are carbon atoms. The carbon atoms are optionally substituted. Carbocyclic aryl groups include monocyclic carbocyclic aryl groups and optionally substituted naphthyl groups.

Heterocyclic aryl groups are groups having from 1 to 3 heteroatoms as ring atoms in the aromatic ring and the remainder of the ring atoms are carbon atoms. Suitable heteroatoms may include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted. A “carbalkoxy” group refers to a COOX group, wherein “X” is an lower alkyl group.

The term “lower” referred to herein in connection with organic radicals or compounds respectively defines such with up to and including 7, and may include one or two carbon atoms. Such groups may be straight chain or branched.

Heterocyclic aryl groups are groups having from 1 to 3 heteroatoms as ring atoms in the aromatic ring and the remainder of the ring atoms are carbon atoms. Heteroatoms may include oxygen, sulfur, and nitrogen, and include furanyl, thienyl, pyridyl, pyrrolyl, N-lower alkyl pyrrolo, pyrimidyl, pyrazinyl, imidazolyl and the like, all optionally substituted.

An “amide” refers to an —C(O)—NH—R, where R may be alkyl, aryl, alkylaryl or hydrogen.

A “thioamide” refers to —C(S)—NH—R, where R may be alkyl, aryl, alkylaryl or hydrogen.

An “ester” refers to an —C(O)—OR′, where R′ may be alkyl, aryl, or alkylaryl.

An “amine” refers to a —N(R″)R′″, where R″ and R′″, may be independently hydrogen, alkyl, aryl, or alkylaryl.

A thioether refers to R—S—R, where R is either alkyl, aryl, or alkylaryl.

An ether refers to R—O—R, where R is either alkyl, aryl, or alkylaryl.

To assist in understanding the present invention, soluble combinatorial libraries of several certain core molecules are described below. The following examples relating to the present invention should not, of course, be construed as specifically limiting the invention, and such variations of the invention, now known or later developed, which would be within the purview of one skilled in this art, are to be considered to fall within the scope of this invention as claimed below.

### EXAMPLE 1

### A Soluble Combinatorial Library of β-Blocker Compositions

Compositions and compounds comprising β-blocker compositions are useful for screening for pharmacologically useful compounds which may be used for diagnostic and/or therapeutic purposes. The present invention allows the synthesis and screening of a large and diverse set of β-blocker compositions. Thus, novel and effective β-blocker compositions that are pharmacologically active can be more rapidly and efficiently identified by the present invention than by conventional methods. A particularly important use of the soluble combinatorial library of β-blocker compositions would be to develop new and effective drugs that affect the autonomic nervous system.

Synthesis of β-blocker compositions using the present invention is a simple efficient process involving basically two steps. Nonetheless, at each step, virtually unlimited diversity can be easily and rapidly introduced into the synthesis, creating a diverse plurality of β-blocker molecules in high yields.

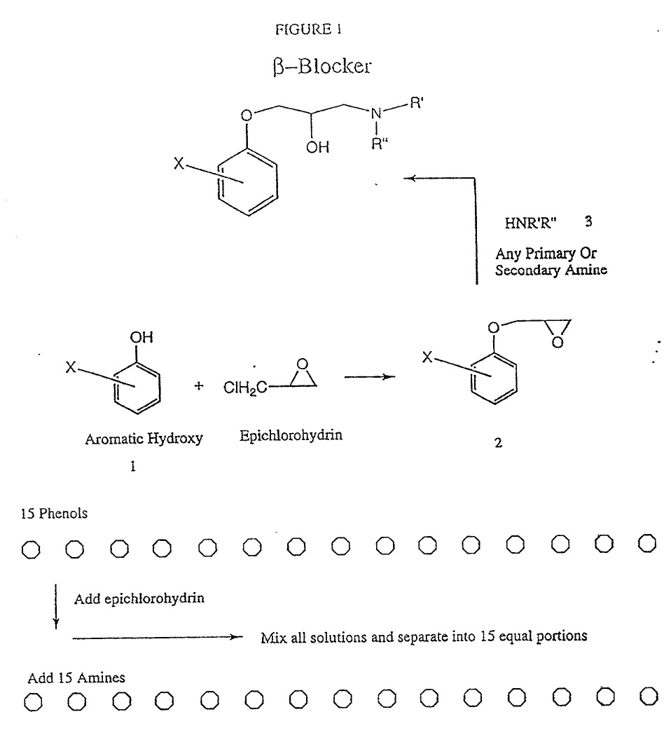

As an example, in one synthetic scheme, a phenol, 1, such as naphthol, is treated with epichlorohydrin. See FIG. 1. The resulting compound, 2, is reacted with an amine, 3, to yield, for example, the β-blocker propranolol.

The present invention allows the rapid synthesis of large set of these β-blockers: At the first step in this synthesis, say, 15 different phenols in 15 separate solutions may be reacted with epichlorohydrin. The Rn group of the phenol may be one or more of the following groups: alkyl, alkenyl, alkynyl alkoxy, aryl, alkylaryl, carboxylic aryl, heterocyclic aryl, carbalkoxy, heterocyclic aryl, amide, thioamide, ester, amine, or thioether. m, as indicated on FIG. 1, may range from 1 to 4. The phenol may have one or more Rn groups. Likewise, the Rp group of the phenol may be one or more of the following groups: alkyl, alkenyl, alkynyl alkoxy, aryl, alkylaryl, carboxylic aryl, heterocyclic aryl, carbalkoxy, heterocyclic aryl, amide, thioamide, ester, amine, or thioether. o, as indicated on FIG. 1, may range from 1 to 4. The phenol may have one or more Rp groups.

All 15 of these solutions may then be pooled and rapidly reacted with 15 different amines, quickly and easily producing 225 different types of β-blockers. The amine, 3, may contain an Rq that is one of the following groups: alkyl, alkenyl, alkynyl alkoxy, aryl, alkylaryl, carboxylic aryl, heterocyclic aryl, carbalkoxy, or heterocyclic aryl. Similarly, The Rr of the amine may be one of the following groups: alkyl, alkenyl, alkynyl alkoxy, aryl, alkylaryl, carboxylic aryl, heterocyclic aryl, carbalkoxy, or heterocyclic aryl. These β-blockers can then be rapidly assayed and characterized.

### EXAMPLE 2

### A Soluble Combinatorial Library of γ-Lactamtide Compositions

A soluble combinatorial library of γ-lactamtide compositions is useful for screening for pharmacologically useful compounds which may be used for diagnostic and/or therapeutic purposes. A preferred use is to use the library of γ-lactamtide compositions to search for molecules that mimic the activity of naturally occurring peptides.

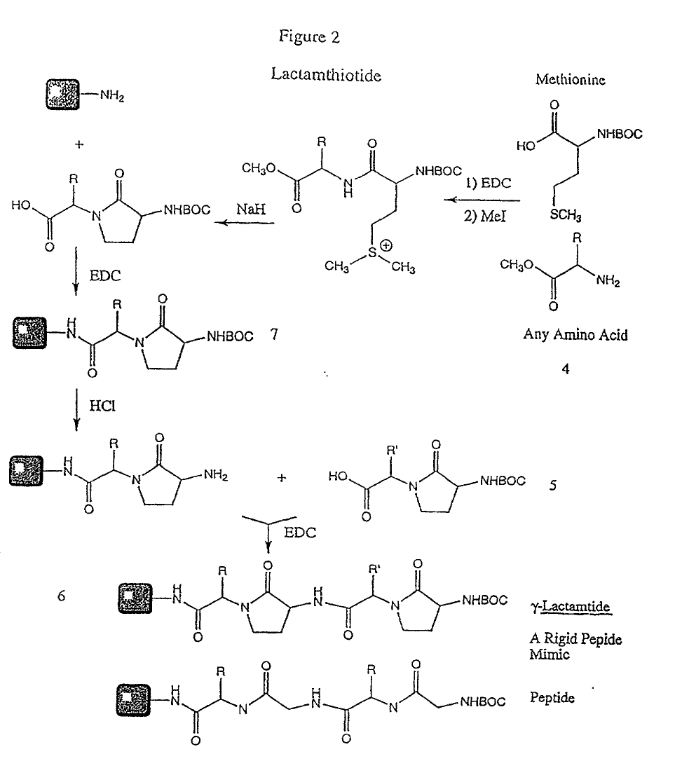

An example of a synthetic scheme for a soluble combinatorial library of γ-lactamtide composition is as follows: Methionine, protected at the amino terminus, is reacted with an amino acid, 4, and treated with EDC followed by MEI. Please see FIG. 2. The methionine protecting group may be of any type well known in the art that permits the lactams to be incorporated into peptides or other target molecules. For example, the protecting group may be t-Boc. Examples of the R group of the amino acid may be hydrogen, CH2CH(CH3)2, CH2Ph, CH2, CH2CH2CH2CH2-NHCbZ, or one of the following groups: alkyl, alkenyl, alkynyl alkoxy, aryl, alkylaryl, carboxylic aryl, heterocyclic aryl, carbalkoxy, heterocyclic aryl, amide, thioamide, ester, amine, or thioether. The R group of the amino acid may also be one of the naturally occurring R groups of the natural amino acids.

The resulting product is induced to cyclization to the lactam by the addition of methyl iodide, causing interalkylation and forming a lactam with a five member ring. This compound is then reacted with the amino terminus of a soluble polymer. The t-Boc blocking group is cleaved from the amino terminus of the resulting product, 7.

The deprotected compound is then reacted with an additional lactam, 5, to form γ-lactamtide. The R″ group of the additional lactam, 5. Examples of the R″ group of the additional lactam, 5, may be hydrogen, CH2CH(CH3)2, CH2Ph, CH2, CH2CH2CH2CH2-NHCbz, or one of the following groups: alkyl, alkenyl, alkynyl alkoxy, aryl, alkylaryl, carboxylic aryl, heterocyclic aryl, carbalkoxy, heterocyclic aryl, amide, thioamide, ester, amine, or thioether. The R″ group of 5 may also be one of the naturally occurring R groups of the natural amino acids.

### EXAMPLE 3

### A Soluble Combinatorial Library of δ-Lactamthiotide Compositions

A combinatorial library of δ-lactamthiotide compositions is useful for screening for pharmacologically useful compounds which may be used for diagnostic and/or therapeutic purposes.

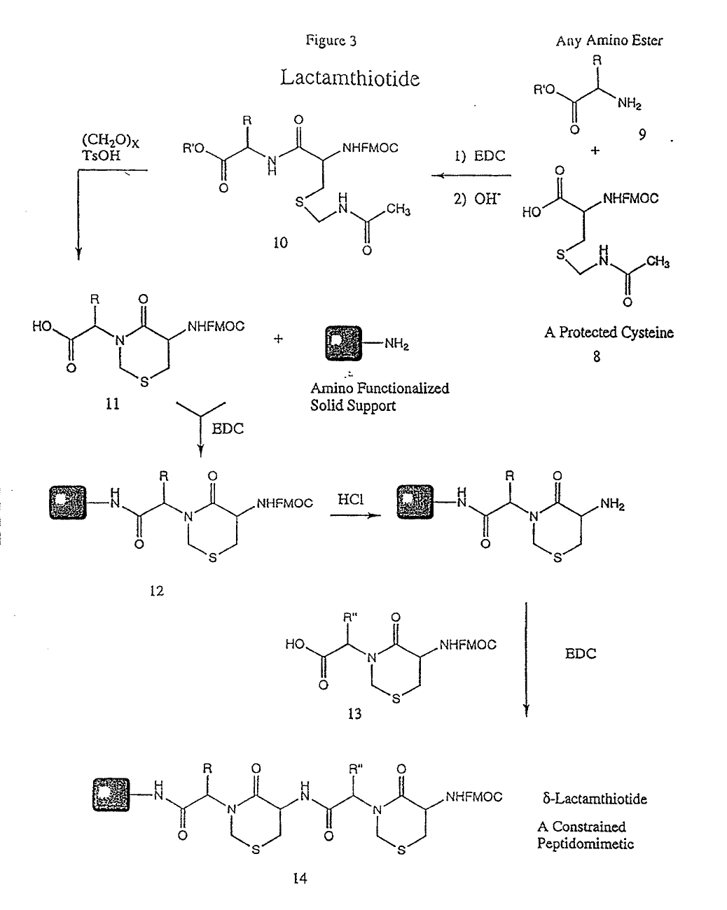

One possible synthesis of this library using the present invention is as follows: Protected cysteine, 8, is reacted with an amino acid ester, 9, and treated first with EDC and second with hydroxide (OH)—. Please see FIG. 3. R′ of the amino acid ester may be one of the following groups: alkyl, alkenyl, alkynyl alkoxy, aryl, alkylaryl, carboxylic aryl, heterocyclic aryl, carbalkoxy, heterocyclic aryl, amide, thioamide, ester, amine, or thioether. The R′ group of 9 may also be one of the naturally occurring R groups of the natural amino acids. The R″ group of the amino ester, 9, may be one of the following groups: alkyl, alkenyl, alkynyl alkoxy, aryl, or alkylaryl.

(CH2O)x and TsDH is added to the resulting compound, 10, in order to induce the compound to cyclization to the five member ring lactam, 11. The lactam, 11, is reacted with the amino terminus of a soluble polymeric compound. The bound lactam, 12, is deprotected, i.e., the protecting group is cleaved from the amino terminus of 12. The deprotected 12 is then reacted with unbound lactam, 13. The R′″ group of the unbound lactam, 13, may be one of the following groups: alkyl, alkenyl, alkynyl alkoxy, aryl, alkylaryl, carboxylic aryl, heterocyclic aryl, carbalkoxy, heterocyclic aryl, amide, thioamide, ester, amine, or thioether. The R′″group of 13 may also be one of the naturally occurring R groups of the natural amino acids. The resulting product of the reaction is bound δ-lactamthiotide, 13, a constrained peptidomimetic.

### EXAMPLE 4

### A Soluble Combinatorial Library of γ-Lactampeptide Compositions

A Soluble Combinatorial Library of γ-Lactampeptide compositions is useful for screening for pharmacologically useful compounds which may be used for diagnostic or therapeutic purposes or both. For example, a soluble combinatorial library of γ-lactampeptide compositions is useful to the discovery of peptide analogues that display the same or enhanced biological activity as the activity of naturally occurring peptides.

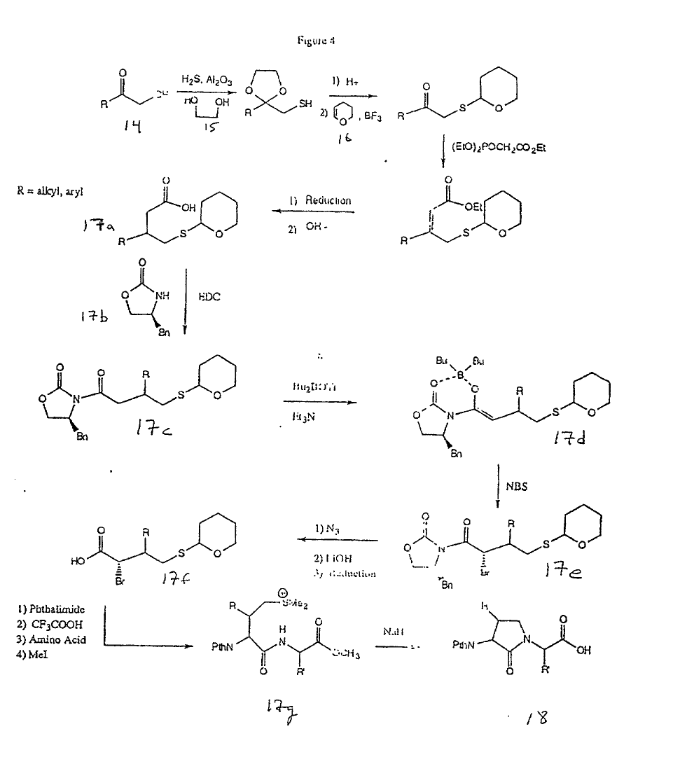

Please see FIG. 4. R(CH2═O)CH3OH, 14, is treated with H2S, Al2O3, and compound 15. R may be an alkyl, aryl, or alkylaryl group. The resulting compound is treated with acid (H+) and then reacted with compound 16 in the presence of BF3. The resulting compound is reacted with (EtO)2POCH2COOEt, and then reduced and treated with hydroxide to yield compound 17a. Compound 17a is reacted with compound 17b and EDC resulting in compound 17c. Compound 17c is reacted with Bu2BOH in the presence of Et3N to yield compound 17d. 17d is treated with NBD producing compound 17e. Compound 17e is reacted with N3 and then with LiOH, and then with a reducing agent to yield compound 17f. Compound 17f is treated first with phthalimide, second with CF3COOH, third with an amino acid and fourth with MeI to yield compound 17g. The R′ group of the amino acid may be hydrogen, alkyl, alkenyl, alkynyl alkoxy, aryl, alkylaryl, carboxylic aryl, heterocyclic aryl, carbalkoxy, heterocyclic aryl, amide, thioamide, ester, amine, or thioether. The R″ group of the amino acid may also be one of the naturally occurring R groups of the natural amino acids. Compound 17g is then treated with NaH to induce cyclization, yielding a γ-lactampeptide, 18. This γ-lactampeptide can serve then be attached to a soluble polymeric support. The attached γ-lactampeptide then chemically may be modified. Also, the attached γ-lactampeptide may be extended by the addition of other γ-lactampeptides, amino acids, or amino acid analogues.

### EXAMPLE 5

### A Soluble Combinatorial Library of Aryloxyacetic Acid Compositions

A combinatorial library of aryloxyacetic acid compositions has wide application and use in the fields of medical, pharmacological and scientific research. In particular, the library of aryloxyacetic compositions may be used to screen for new drugs that target atherosclerosis receptors. In addition, the aryloxyacetic compositions may be useful for screening for effective novel drugs which lower triglyceride levels.

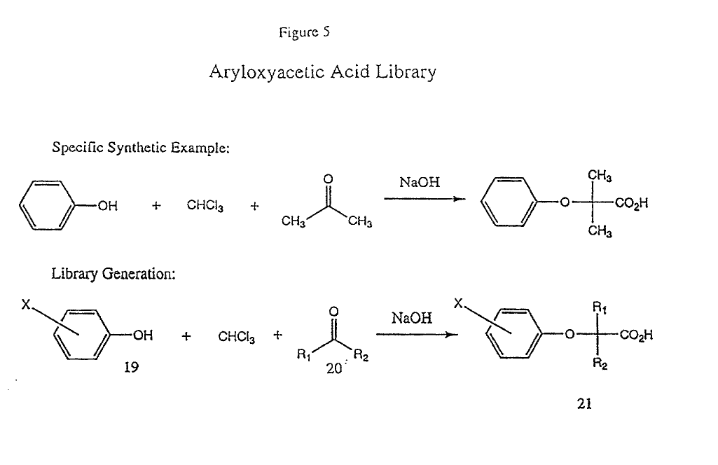

In one possible one step synthesis of a combinatorial library of aryloxyacetic compositions, a substituted phenol, 19, is reacted with a ketone or aldehyde, 20, in a suitable solvent in the presence of base. Please see FIG. 5. The base may be NaOH. The solvent may be methylene chloride. The phenol, 19, may be substituted at either the ortho, meta or para position, or at a combination of these positions. The X group of 19 may be one of the following groups: hydroxy, halogen, alkyl, alkenyl, alkynyl alkoxy, aryl, alkylaryl, carboxylic aryl, heterocyclic aryl, carbalkoxy, heterocyclic aryl, amide, thioamide, ester, amine, thioether, or fused. The phenol, 19, may have multiple X groups. The R1 group of compound 20 may be hydrogen, or may be one of the following groups: alkyl, alkenyl, alkynyl alkoxy, aryl, alkylaryl. Similarly, the R2 group of compound 20 may be hydrogen, or may be one of the following groups: alkyl, alkenyl, alkynyl alkoxy, aryl, alkylaryl. Gilman and Wilder, (1955) is incorporated by reference. The resulting compound is an aryloxyacetic acid, 21.

### EXAMPLE 6

### A Soluble Combinatorial Library of Polyether Backbone Compositions

A combinatorial library of polyether backbone compositions is useful for screening for pharmacologically useful compounds which may be used for diagnostic and/or therapeutic purposes. In particular the polyether backbone compositions may have lipophilic qualities that allow the compositions to cross through a patient's cell membranes.

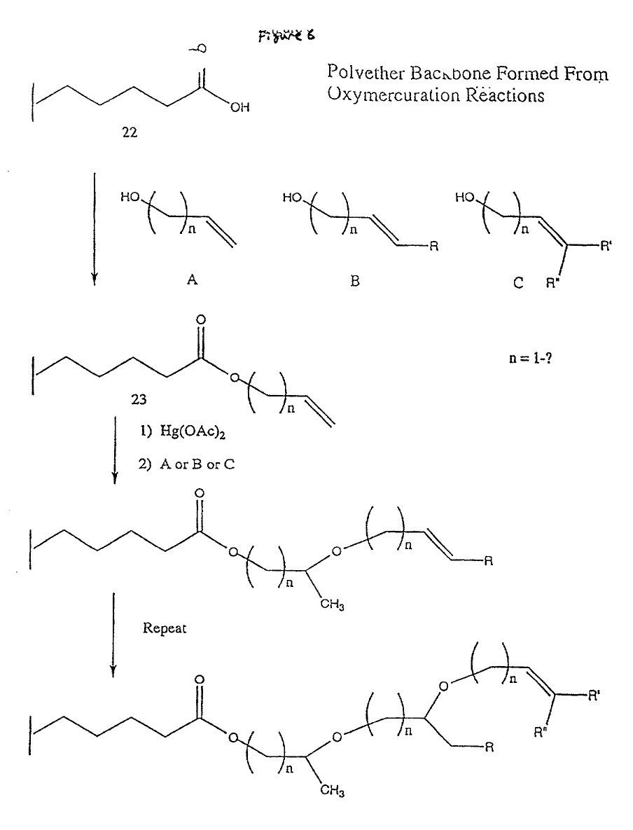

Please see FIG. 6. Compound 22 is esterified with a vinyl selected from the group consisting of the vinyls A, B. and C. n may vary on compounds A, B and C, with n equal to whole number greater than zero. The reaction yields compound 23. Compound 23 is treated with Hg(OAc)2 and then reacted with a vinyl compound selected from the group consisting of A, B and C. n may vary on compounds A, B and C, with n equal to a whole number greater than zero. The reaction step of treating Hg(OAc)2 and reacting with a vinyl from the group consisting of compounds A, B, and C is repeated until a polyether compound of the desired size is created.

### EXAMPLE 7

### A Soluble Combinatorial Library of Polyoxygenated Compositions

A soluble combinatorial library of polyoxygenated compositions is useful for screening for pharmacologically useful compounds which may be used for diagnostic or therapeutic purposes or both.

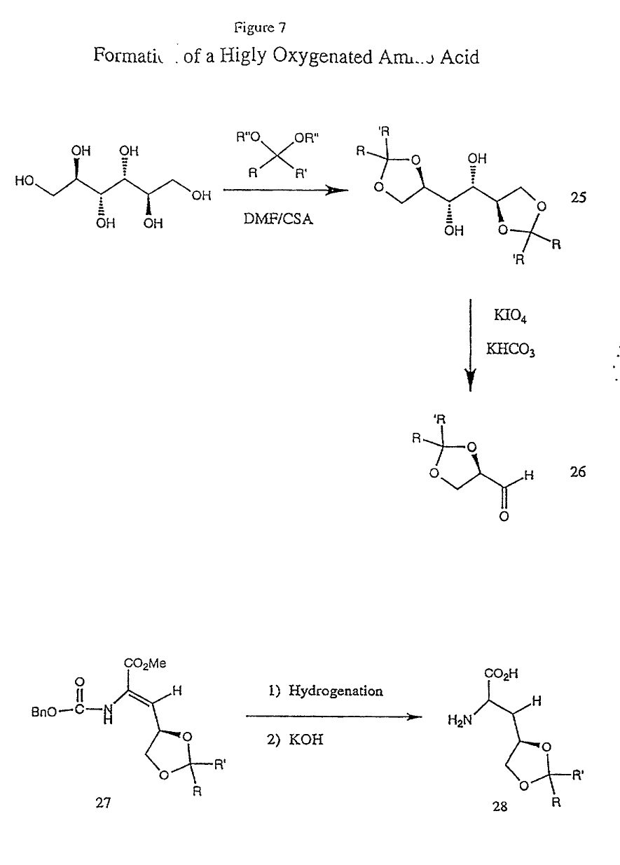

A soluble combinatorial library of polyoxygenated amino acids may also be synthesized as follows: Please see FIG. 7. Condensation of mannitol with a compound 24 is carried out in a suitable solvent. Mannitol may be d or l mannitol. d-mannitol is a preferred form. The Rc and Rd groups of compound 24 may be any combination of alkyl or alkylaryl. The Ra and Rb groups of compound 24 may be any combination of hydrogen, alkyl, alkenyl, alkynyl alkoxy, aryl, alkylaryl. Dimethoxyketal is a preferred form of compound 24. The solvent may be Dimethylformamide and CSA. The resulting diol, 25, is cleaved under periodic oxidation (KIO4 and KCHO3) to form a highly oxygenated aldehyde, 26. The synthesis of alkylidene protected glyceraldehydes is well known in the art and has been reported in the following publications, which are incorporated by reference: Schmid & Bradley, Synthesis (1992), and Schmid, et al., J. Org. Chem. (1991). Condensation of the ketalized glyceraldehyde, 27, with a dialkylphosphinyl compound yields an amino ester, 28. This amino ester may be hydrogenated and then treated with potassium hydroxide to yield a highly oxygenated amino acid. The amino acid may then be protected at the amino terminus using, for example, FMOC.

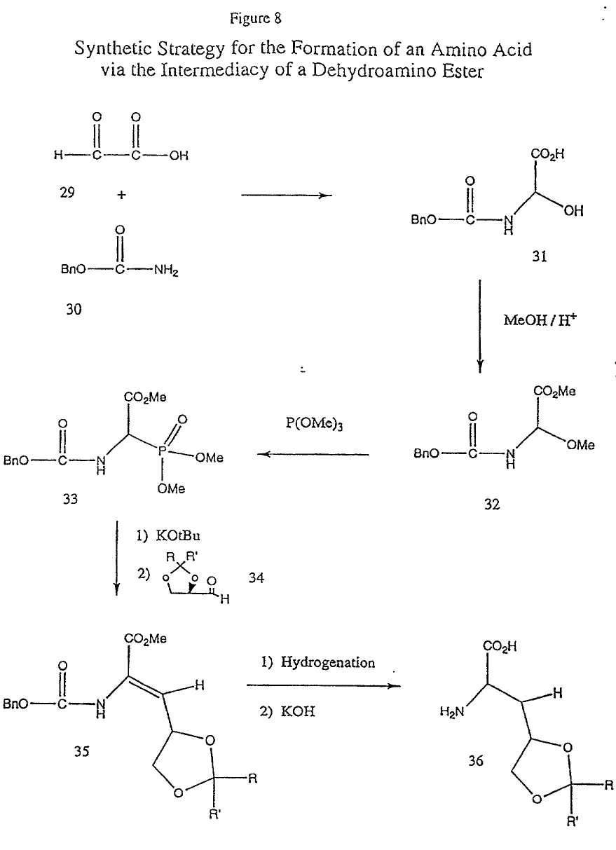

A soluble combinatorial library of polyoxygenated amino acids may also be synthesized as follows: Please see FIG. 8. Zoller & Ben-Ishai, Tetrahedron 1975, and Schmidt, et al., Synthesis 1984 are incorporated by reference. Glyoxalic acid, 29, is reacted with compound 30 to yield compound 31. Compound 31 is then treated with acid, in a suitable solvent. The solvent may be methanol. The resulting compound, 32, is reacted with P(OMe)3 to yield compound 33. Compound 33 is reacted with KotBu and then compound 34, to form a dehydroamino ester 35. The R′ and R″ groups of compound 34 may be a combination of: hydrogen, alkyl, alkenyl, alkynyl alkoxy, aryl, alkylaryl. The ester is hydrogenated and then treated with base to yield a highly oxygenated amino acid, 36. The base may be potassium hydroxide. The highly oxygenated amino acid, 36, may then be protected at the amino terminus using, for example, FMOC.

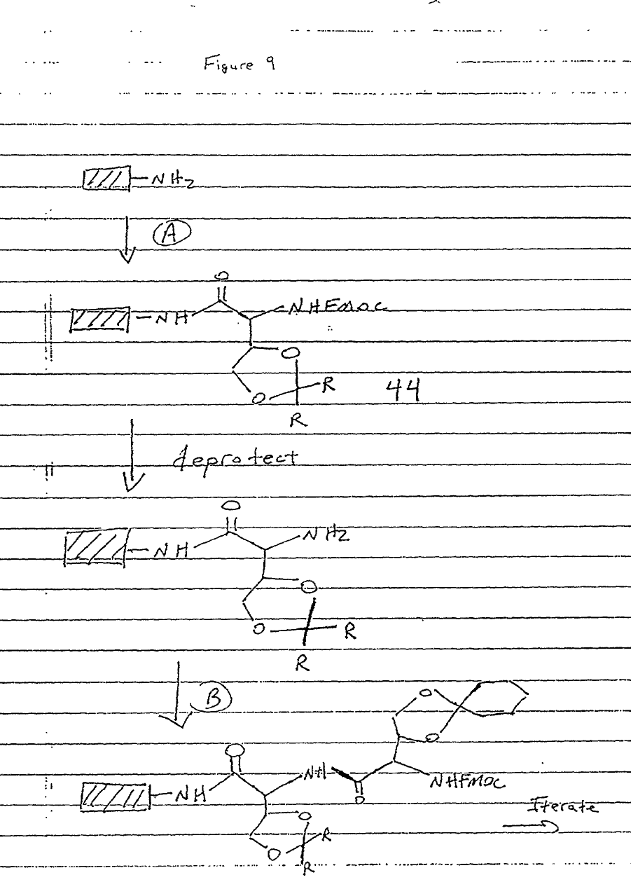

Synthesis of the polyoxygenated library from the highly oxygenated amino acids may be accomplished as follows: Please see FIG. 9. A highly oxygenated amino acid, A, is coupled to the amino terminus of a soluble polymeric compound. The coupled amino acid, 44, is deprotected, and then reacted with an additional highly oxygenated amino acid. Additional highly oxygenated amino acids are added until a peptide backbone having the desired number of amino acids is produced.

### EXAMPLE 8

### A Soluble Combinatorial Library of Triazine-dione Compositions

A soluble combinatorial library of triazine-dione compositions is useful for screening for pharmacologically useful compounds which may be used for diagnostic or therapeutic purposes or both.

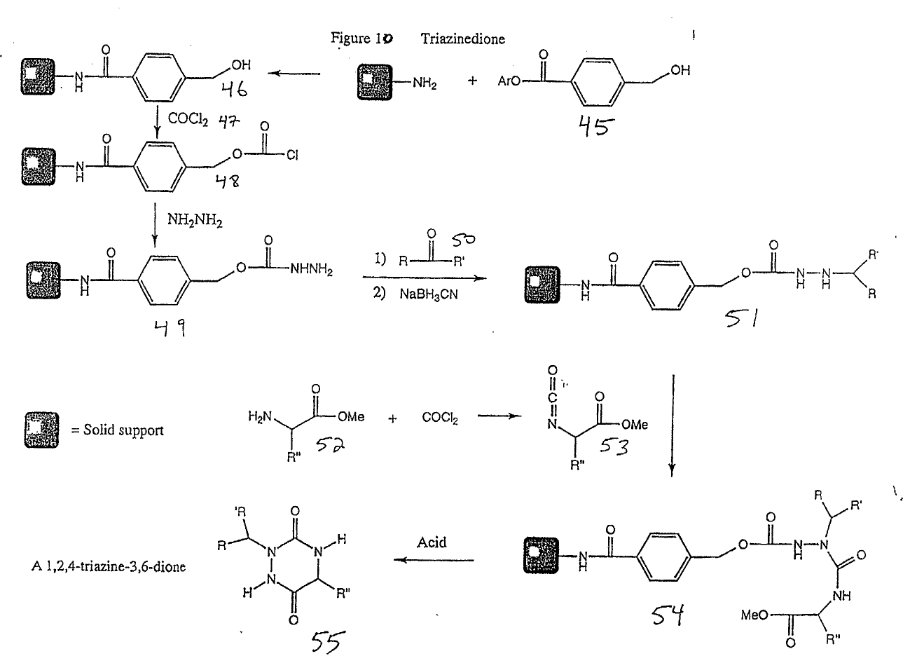

Please see FIG. 10. Compound 45 is linked to a soluble polymeric compound. The resulting compound 46 is reacted with compound 47, an ester, to yield compound 48. Compound 48 is reacted with NH2—NH2. The resulting compound 49 is reacted with compound 50 to yield compound 51. The Rh and Ri groups of compound 50 may be any combination of the following: hydrogen, alkyl, alkenyl, alkynyl alkoxy, aryl, alkylaryl. Compound 51 is then reacted with compound 53. Compound 53 may be prepared conveniently by treating compound 52 with phosgene. The R′ group of compound 52 may be alkyl, alkenyl, alkynyl alkoxy, aryl, alkylaryl, carboxylic aryl, heterocyclic aryl, carbalkoxy, heterocyclic aryl, amide, thioamide, ester, amine, or thioether. The R′ group of 52 may also be one of the naturally occurring R groups of the natural amino acids. The reaction between compounds 51 and 53 yields compound 54. Compound 54 is then treated with acid to yield 1,2,3-triazine-3,6-dione, 55.

### EXAMPLE 9

### A Soluble Combinatorial Library of Dideoxynucleotides Compositions

A soluble combinatorial library of dideoxynucleotides is useful for screening for pharmacologically useful compounds which may be used for diagnostic or therapeutic purposes or both. For example, the dideoxynucleotides produced by the present invention can be used to produce and screen a large and diverse set of dideoxynucleotides to be used for gene vectors, gene therapy, gel assays and the like.

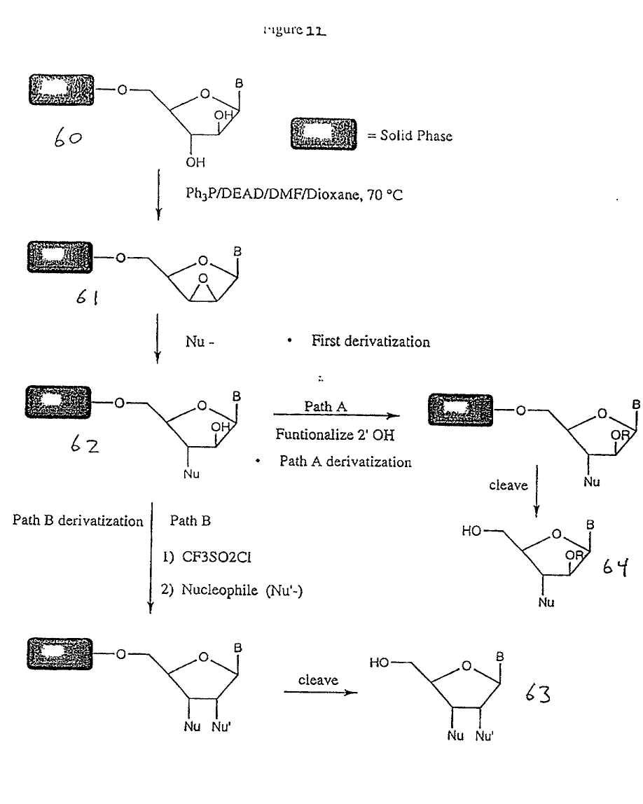

Chen, et al., The Journal of Organic Chemistry (1991) is incorporated by reference. Please see FIG. 11. Compound 60 is reacted with Ph3P, ETO2CN═NCO2ET in a suitable solvent. The solvent may be dimethyformamide and dioxane at 70 degrees Celsius. A preferred forms of the solid phase of compound 60 are polystyrene, tentagel and control pore glass. Alternatively, instead of a solid phase, one may use a soluble polymeric compound. The B of compound 60 may be a dideoxynucleotide base, such as Adenine, Guanine, Cytosine, Thymine, or Uracil, or an unnatural purine or pyrimidine heterocyclic compound. The product, 61, is then reacted with a nucleophile. The nucleophile may be an amino acid, a sugar, a small molecule such as, but not limited to, N3 or NaI, or OR, SR, NR1R2, CN, Alkyl, N3, Halide, Phosphate ester, or sulfate ester. The R, R1 and R2 groups may be alkyl, aryl, acyl, phosphate or sulfate.

The resulting product 62 may be treated with CF3SO2Cl, reacted with a nucleophile, Nu′, and then cleaved from the bead to yield compound 63. The nucleophile Nu′ may be an amino acid, a sugar, a small molecule such as, but not limited to, N3 or NaI, or OR, SR, NR1R2, CN, Alkyl, N3, Halide, Phosphate ester, or sulfate ester. The R, R1 and R2 groups may be alkyl, aryl, acyl, phosphate or sulfate.

Compound 62 may also be capped at the 2′-OH and then cleaved from the bead to yield compound 64.

### EXAMPLE 10

### A Soluble Combinatorial Library of Lycoramine-like Nucleus Compositions

A soluble combinatorial library of lycoramine-like nucleus compositions is useful to many important research efforts, including medical, pharmacological and scientific research efforts. For example, a soluble combinatorial library of lycoramine-like nucleus compositions may be useful as antimicrobial agents, analgesics, and as hallucinatory agents.

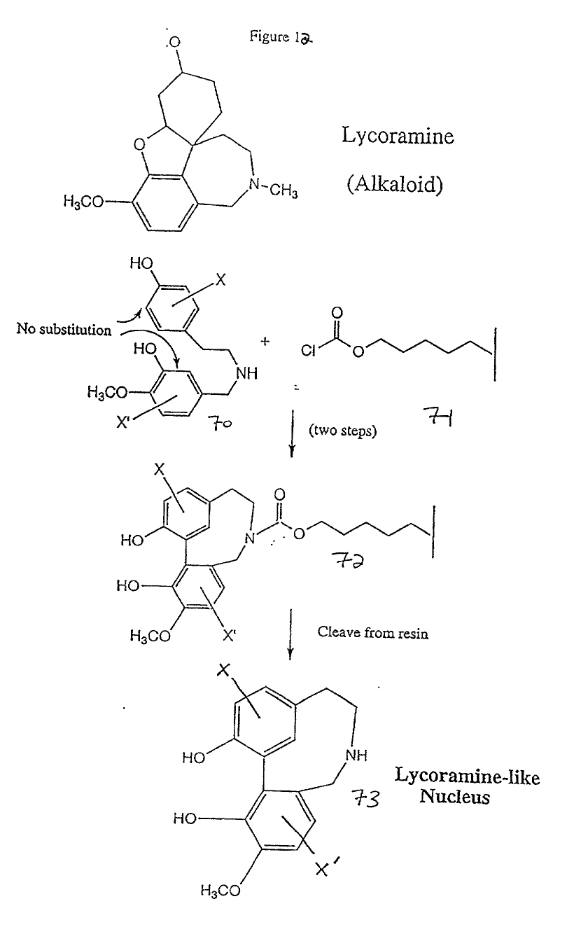

Please see FIG. 12. Compound 70 is reacted with compound 71, which may be attached to a resin or soluble polymeric compound, to yield compound 72. Compound 70 may be substituted. The R′ and R″ groups may be any combination of: alkyl, alkenyl, alkynyl alkoxy, aryl, alkylaryl, carboxylic aryl, heterocyclic aryl, carbalkoxy, heterocyclic aryl, amide, thioamide, ester, amine, or thioester. x may be 1, 2 or 3. y may be 1 or 2. Compound 73 is then cleaved from the resin to yield compound 73, a lycoramine-like nucleus.

### EXAMPLE 11

### A Soluble Combinatorial Library of β-Lactam Compositions

A soluble combinatorial library of β-lactam compositions is useful in the medical, pharmacological and scientific arts.

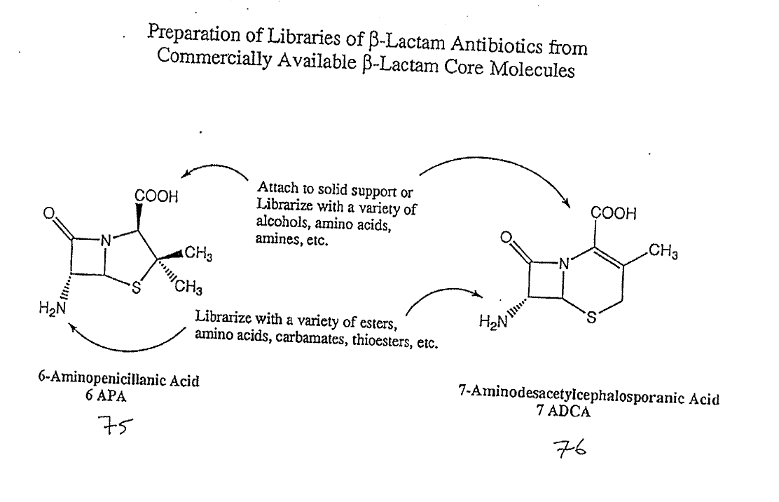

A soluble combinatorial library of β-lactam compositions, FIG. 13, may be conveniently synthesized by attaching the carboxyl terminus of compound 75 or 76 to a soluble polymeric support. The amino terminus of compound 75 or 76 may then be reacted with an amino acid, carbamate, amide, thioamide, ester, amine, or thioester.

### EXAMPLE 12A

### A Soluble Combinatorial Library of α-Azetide Compositions

A soluble combinatorial library of α-azetide compositions is useful for medical, pharmacological and medical research. For example, a soluble combinatorial library of α-azapeptides are useful as inhibitors and active site titrants of a number of enzymes, including human leukocyte elastase, porcine pancreatic elastase, chymotrypsin-like enzymes, and cysteine protease. The α-azetide library may also be useful as analogues of norophthalmic acid amide.

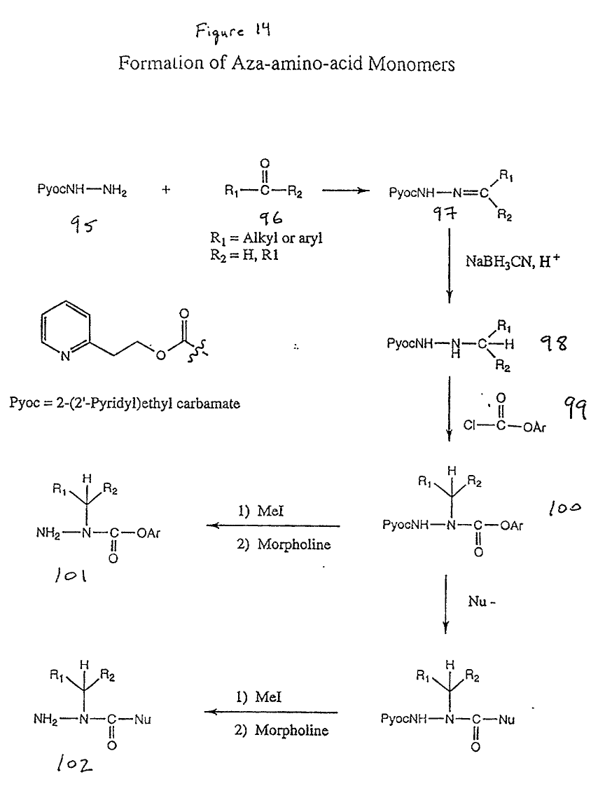

An example synthesis of a soluble combinatorial library of α-aza amino acid compositions is given in FIG. 14. A carbazate, compound 95, is reacted with compound 96 to yield compound 97. R2 of compound 96 may be hydrogen, alkyl, alkenyl, alkynyl alkoxy, aryl, or alkylaryl. R1 of compound 96 may be alkyl, alkenyl, alkynyl alkoxy, aryl, or alkylaryl. Compound 97 is then reacted with NaBH3CN and acid to yield compound 98. Compound 98 is then reacted with compound 99 to yield compound 100. Compound 100 is then treated with MeI and then with morpholine resulting in compound 101, an aza-amino-acid. Alternatively, compound 100 is reacted with a nucleophile. The resulting compound is then reacted with MeI followed by morpholine to yield compound 102, an aza-amino-acid.

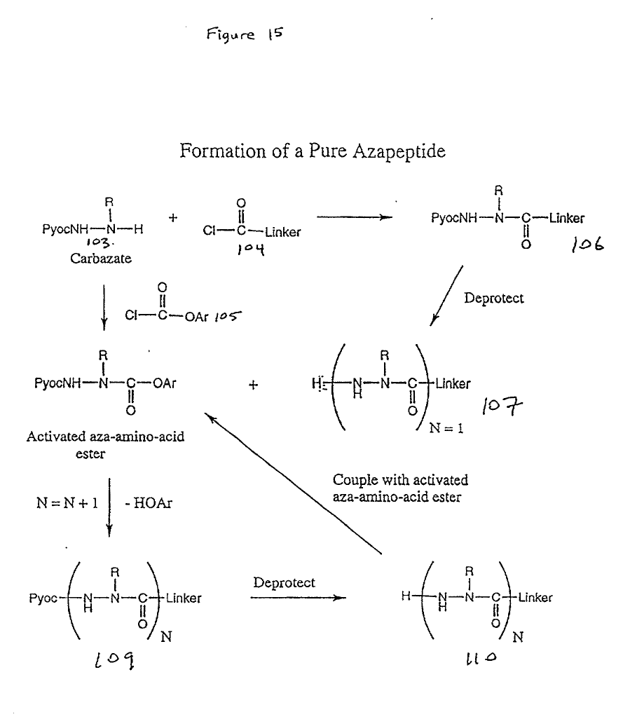

In another synthetic scheme, see FIG. 15, compound 103 is reacted with compound 104 to yield compound 106. The linker of compound 104 may be an aliphatic group, such as an aldehyde, for example acetaldehyde or cyclohexanecarboxaldehyde, a ketone, or methacrolein; or an aromatic, such as phenylacetaldehyde, furfural, 2,4,6,-trimethoxybenzaldehyde or piperonal; or a charged compound such as 5-formyl-2-furansulphonic acid or pyridine-2-carboxaldehyde-N-oxide. Compound 106 is deprotected to yield compound 107. Compound 107 is then reacted with an activated aza-amino acid ester, compound 108, to yield compound 109, step *. Compound 108 may be conveniently prepared by reacting a carbazate, compound 103, with compound 105. Compound 109 may then be deprotected and step * repeated to extend the chain of azapeptides to the desired length.

### EXAMPLE 12B

### Method of Producing a Combinatorial Library of α-Azetide Compositions.

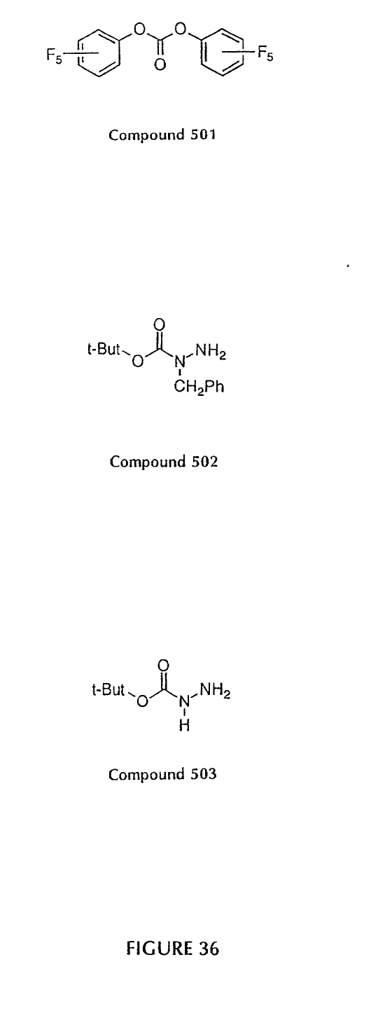

Preparation of Compound 501: Synthesis of Bispentafluorophenol Carbonate (Structure of Compound 501 is Depicted on FIG. 36).

Pentafluorophenol (0.27 mol.; commercially available from Aldrich chemical) was dissolved in 0.5 Molar KOH and cooled to 0° C. Phosgene was then passed through this solution with vigorous mixing. The pH of the reaction mixture was controlled to be no less than 6.0. Sometimes the carbonate crystallized from solution, but more often an oily precipitate formed. Next, the reaction mixture was kept at 0° C. overnight. The solidified residue was filtered off, washed with water and dissolved in chloroform. The solution was dried over anhydrous sodium sulfate, filtered and evaporated. The crude crystalline product, with a strong, chloroformate-like odor from an impurity, was recrystallized from hexane. The yield was approximately 75%, starting with 55 grams pentafluorophenol.

Preparation of Compound 502 (Structure of Compound 502 is Depicted on FIG. 36).

A solution of 0.098 mole of benzyl chloride in 0.98 Molar of ethanol was added over a period of 1 hour to a refluxing solution of 0.51 mol of 85% hydrazine hydrate (commercially available from Aldrich company) in 2.55 Molar of ethanol. After a reflux period of 6 hours, the alcohol was removed by distillation. The residue was extracted with ether, the ethereal extracts dried with potassium carbonate and filtered. The crude base, 1.0 equivalents, was next exposed to 1.1 equivalents of ditert-butyl dicarbonate (commercially available from Aldrich company) in 0.1 Molar methylene chloride and then stirred overnight at 25° C. The solvent was next removed by distillation and the remaining residue was extracted with ether, washed in water (1×) and dried over potassium carbonate and filtered. Product can then be purified by flash chromatography using a methylene chloride: ether/petroleum ether gradient.

Preparation of Compound 503 (Structure of Compound 503 is Depicted on FIG. 36).

A solution of 85% hydrazine hydrate (10 equivalents; commercially available from Aldrich company) in 2.55 Molar of ethanol was exposed to 1.0 equivalents of di-tert-butyl dicarbonate (commercially available from Aldrich company) and then stirred overnight at 25° C. The solvent was next removed by distillation and the remaining residue was extracted with ether, washed in water (1×) and dried over potassium carbonate and filtered. Production can then be purified by flash chromatography using a methylene chloride: ether/petroleum ether gradient.

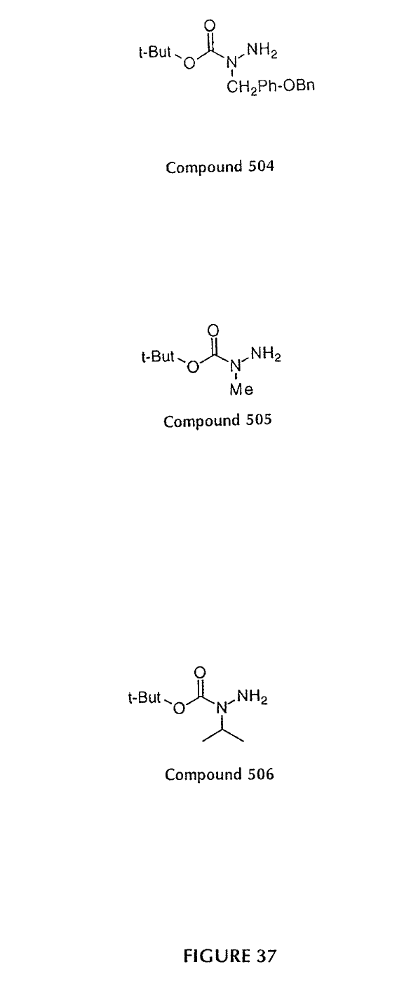

Preparation of Compound 504 (Structure of Compound 504 is depicted on FIG. 37).

To a solution of 1.0 equivalent of p-hydroxybenzylbromide in methylene chloride was added 1.1 equivalents of 60% sodium hydride at 0° C. and allowed to stir for 1 hour. Next, 1.1 equivalents of benzyl bromide was added and then mixture was allowed to stir overnight. The mixture was then quenched with water, diluted with ether and purified by distillation. 1.0 equivalents of the compound was next added over a period of 1 hour to a refluxing solution of 0.51 mol of 85% hydrazine hydrate (commercially available from Aldrich company) in 2.55 Molar of ethanol. After a reflux period of 6 hours, the alcohol was removed by distillation. The residue was extracted with ether, the ethereal extracts dried with potassium carbonate and filtered. The crude base, 1.0 equivalents, was next exposed to 1.1 equivalents of di-tert-butyl dicarbonate (commercially available from Aldrich company) in 0.1 Molar methylene chloride and then stirred overnight at 25° C. The solvent was next removed by distillation and the remaining residue was extracted with ether, washed in water (1×) and dried over potassium carbonate and filtered. Production can then be purified by flash chromatography using a methylene chloride: ether/petroleum ether gradient.

Preparation of Compound 505 (Structure of Compound 505 is depicted on FIG. 37).

A solution of 0.098 mole of methyl iodide in 0.98 Molar of ethanol was added over a period of 1 hour to a refluxing solution of 0.51 mol of 85% hydrazine hydrate (commercially available from Aldrich company) in 2.55 Molar of ethanol. After a reflux period of 6 hours, the alcohol was removed by distillation. The residue was extracted with ether, the ethereal extracts dried with potassium carbonate and filtered. The crude base, 1.0 equivalents, was next exposed to 1.1 equivalents of ditert-butyl dicarbonate (commercially available from Aldrich company) in 0.1 Molar methylene chloride and then stirred overnight at 25° C. The solvent was extracted with ether, washed in water (1×) and dried over potassium carbonate and filtered. Product can then be purified by flash chromatography using a methylene chloride: ether/petroleum ether gradient.

Preparation of Compound 506 (Structure of Compound 506 is Depicted on FIG. 37).

A solution of 0.098 mole of 2-chloropropane in 0.98 Molar of ethanol was added over a period of 1 hour to a refluxing solution of 0.51 mol of 85% hydrazine hydrate (commercially available from Aldrich company) in 2.55 Molar of ethanol. After a reflux period of 6 hours, the alcohol was removed by distillation. The residue was extracted with ether, the ethereal extracts dried with potassium carbonate and filtered. The crude base, 1.0 equivalents, was next exposed to 1.1 equivalents of ditert-butyl dicarbonate (commercially available from Aldrich company) in 0.1 Molar methylene chloride and then stirred overnight at 25° C. The solvent was next removed by distillation and the remaining residue was extracted with ether, washed in water (1×) and dried over potassium carbonate and filtered. Product can then be purified by flash chromatography using a methylene chloride: ether/petroleum ether gradient.

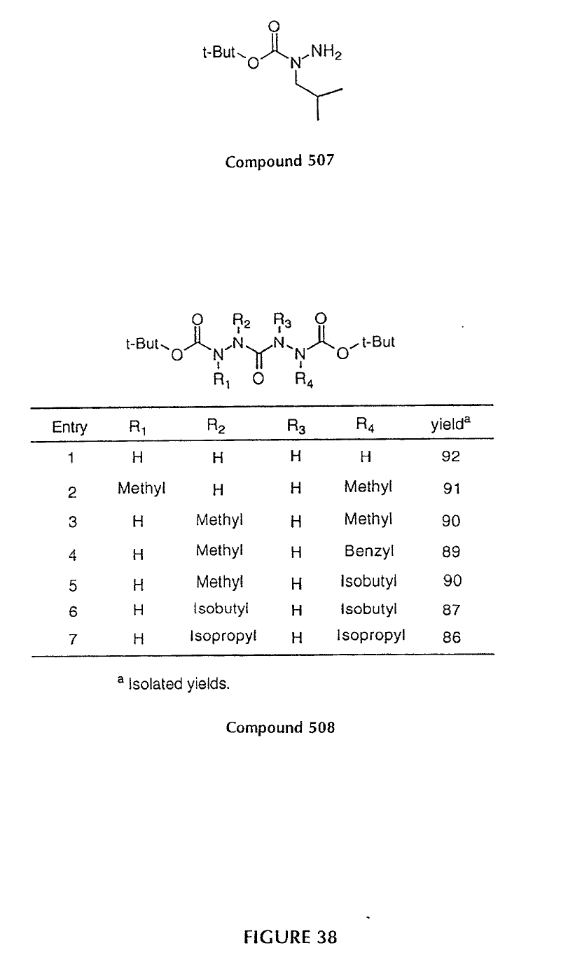

Preparation of Compound 507 (Structure of Compound 507 is Depicted on FIG. 38).

A solution of 0.098 mole of 1-bromo-2-methylpropane (from Aldrich company) in 0.98 Molar of ethanol was added over a period of 1 hour to a refluxing solution of 0.51 mol of 85% hydrazine hydrate (commercially available from Aldrich company) in 2.55 Molar of ethanol. After a reflux period of 6 hours, the alcohol was removed by distillation. The residue was extracted with ether, the ethereal extracts dried with potassium carbonate and filtered. The crude base, 1.0 equivalents, was next exposed to 1.1 equivalents of di-tert-butyl dicarbonate (commercially available from Aldrich company) in 0.1 Molar methylene chloride and then stirred overnight at 25° C. The solvent was next removed by distillation and the remaining residue was extracted with ether, washed in water (1×) and dried over potassium carbonate and filtered. Production can then be purified by flash chromatography using a methylene chloride: ether/petroleum ether gradient.

Preparation of Compound 508 (Structure of Compound 508 is is Depicted on FIG. 38).

A generalized procedure for the synthesis of the azadipeptides (compound 508, entries #1-7, FIG. 38) is as follows. 1.0 equivalent of the alkyl/arylhydrazine (compounds 502 through 507, FIGS. 36, 37 and 38) is added dropwise via syringe pump over a period of 30-40 minutes to compound 501, bispentaflourophenol carbonate (1.1 equivalent) and 1.1 equivalent dimethylaminopyridine (DMAP) in 0.10 Molar methylene chloride at 25° C. After completion of addition, the reaction mixture is exposed to a second 1.1 equivalent of dimethylaminopyridine (DMAP) and another 1.0 equivalent of alkyl/arylhydrazine (compounds 502 through 507, FIGS. 36, 37 and 38), added in one step. After 24 hours, the reaction mixture is evaporated to remove all solvent. The crude product is resuspended in a minimum amount of methylene chloride and purified by flash chromatography in a 9:1 methylene chloride:ether gradient. Typical yields are approximately 85%. See chart at FIG. 38 for variations and yields obtained (unoptimized).

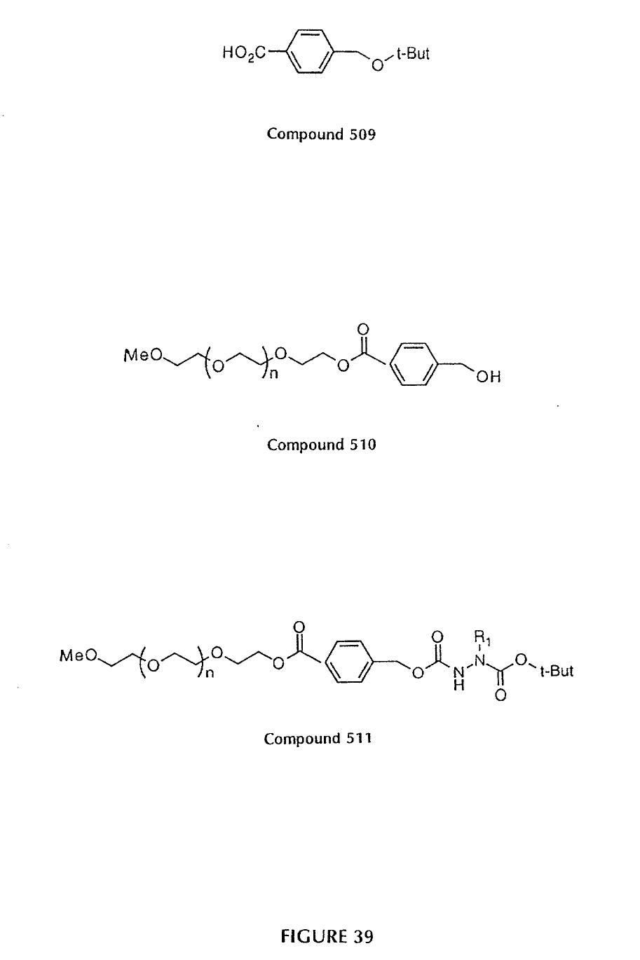

Preparation of Compound 509 (Structure of Compound 509 is Depicted on FIG. 39).

To a solution of methyl 4-(hydroxymethyl)benzoate (2.0 g, 12 mmol, 1.0 equivalents, commercially available from Aldrich company) in 0.10 Molar diethylether, was bubbled 8 mL of isobutylene (2-methylpropene, commercially available from Aldrich company) at −78° C. Next, 10 drops of sulfuric acid were added and the mixture was allowed to stir overnight. The reaction mixture was diluted with ether (25 mL), quenched with sodium bicarbonate (10 mL), washed with water (10 mL), condensed and dried over magnesium sulfate. The product can be purified by flash chromatography or distillation. The production is next exposed to 5 equivalents of LiOH-H₂O in a 3:1 mixture of methanol water (3 Molar). The mixture is allowed to stir for 2 hours at 25° C. and then is extracted with ether and acidified with 1 mL of HCl. The precipitate is collected on a glass filter and can be further purified by flash chromatography or crystallization.

Preparation of Compound 510 (Structure of Compound 510 is Depicted on FIG. 39).

To a solution of (MeO-PEG-OH, 500 MW, commercially available from Sigma Company) in 17 mM of methylene chloride at 25° C., is added 3.0 equivalents of compound 509 (FIG. 39), 3.0 equivalents of 1,3 dicyclohexyl carbodiimide (DCC) and 0.75 equivalents of 4-DMAP (4-dimethylaminopyridine). The reaction mixture is allowed to stir overnight. Next, the mixture is exposed to 3.0 equivalents of triflouroacetic acid (TFA) and allowed to stir an additional 11 minutes at 25° C. The mixture is then poured into ice-cold ether (approximately 17 mM) to precipitate the PEG and then washed with cold ether and ethanol fractions. The final production can be further purified by crystallization from hot ethanol.

Preparation of Compound 511 (Structure of Compound 511 is Depicted on FIG. 39).

Step 1: Formation of the activated azacarbamate. 1.0 equivalent of an alkyl/arylhydrazine (i.e., compounds 502-507, FIGS. 36, 37 and 38) is added dropwise via syringe pump over a period of 30-40 minutes to compound 501 (FIG. 36), bispentaflourophenol carbonate (1.1 equivalent) and 1.1 equivalent dimethylaminopyridine (DMAP) in 0.10 Molar methylene chloride at 25° C. The activated azacarbamate is then further purified by flash chromatography, distillation or crystallization.

Step 2: Coupling of the activated azacarbamate to the PEG support. To 1.0 equivalent of the PEG support (compound 510, FIG. 39) in 17 mM methylene chloride at 25° C., is added 5.0 equivalents of the activated azacarbamate (made in the above step 1) and 5.1 equivalents of 4-dimethylaminopyridine (4-DMAP). The reaction mixture is then allowed to stir for 24 hours and is next precipitated with the addition of ether (17 mM diethyl ether). The production is then further purified by washing with ether (1×) and cold ethanol (1×).

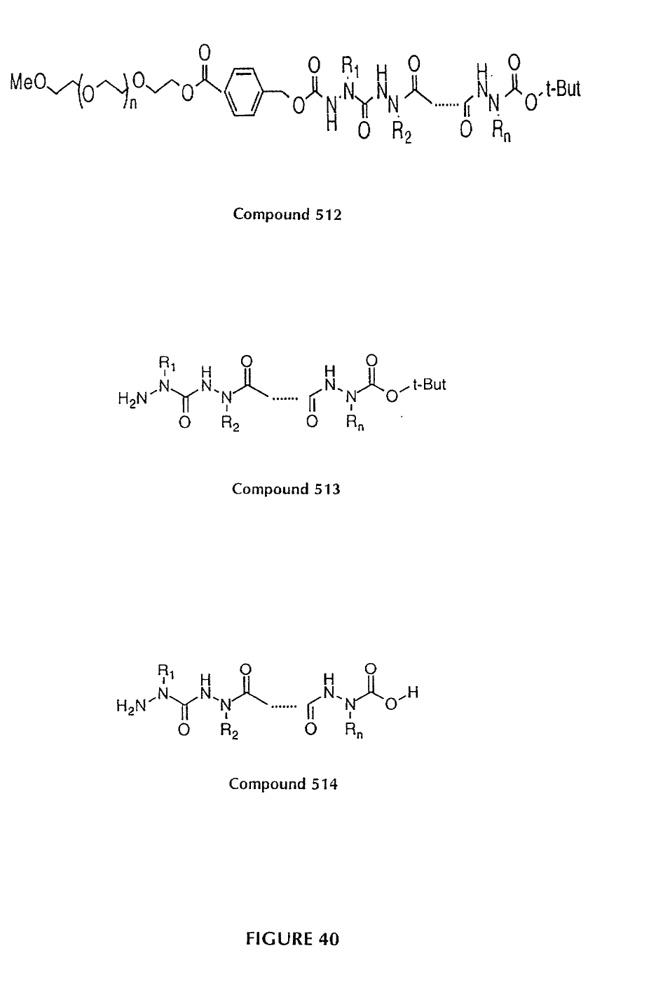

Preparation of Compound 512 (Structure of Compound 512 is Depicted on FIG. 40).

Compound 512 is formed form an iterative cycle of steps 1 through 3 as outlined below.

Step 1: Removal of the t-But protective group. 1.0 gram of compound 511 (FIG. 39) is exposed to a 10% triflouroacetic acid/methylene chloride solution (10 mL, 1:1 TFA/methylene chloride) and allowed to stir at 25° C. for 1 hour. The reaction mixture is next precipitated with the addition of ether (17 mM diethyl ether). The product is then further purified by washing with ether (1×) and can be crystallized from ethanol (1×).

Step 2: Formation of the activated azacarbamate. 1.0 equivalent of an alkyl/arylhydrazine (i.e., compounds 502 through 507, FIGS. 36, 37 and 38) is added dropwise via syringe pump over a period of 30-40 minutes to compound 501 (FIG. 36), bispentaflourophenol carbonate (1.1 equivalent) and 1.1 equivalent dimethylaminopyridine (DMAP) in 0.10 Molar methylene chloride at ° C. The activated azacarbamate is then further purified by flash chromatography, distillation or crystallization.

Step 3: Coupling of the activated azacarbamate to the PEG support. To 1.0 equivalent of the PEG support (compound 510, FIG. 39) in 17 mM methylene chloride at 25° C., is added 5.0 equivalents of the activated azacarbamate (made in the above step 1) and 5.1 equivalents of 4-dimethylaminopyridine (4-DMAP). The reaction mixture is then allowed to stir for 24 hours and is next precipitated with the addition of ether (17 mM diethyl ether). The production is then further purified by washing with ether (1×) and can be recrystallized form hot ethanol.

Step 4: Repeat steps 1 through 3 as desired.

Preparation of Compound 513 (Structure of Compound 513 is Depicted on FIG. 40).

This step removes the aza-peptide from the PEG support and additionally removes the benzyl protecting groups. To 1.0 gram of compound 512 (FIG. 40) in 10 mL methanol at 25° C. is added 200 mg of 10% Pd/C. The reaction mixture is capped with a hydrogen balloon and allowed to stir overnight. The product is washed with ether, filtered and condensed. Further purification can be achieved by standard chromatographic methodologies for small peptides.

Preparation of Compound 514 (Structure of Compound 514 is Depicted on FIG. 40).

Removal of the t-But protecting group. 1.0 gram of compound 513 (FIG. 40) is exposed to a 10% triflouroacetic acid/methylene chloride solution (10 mL, 1:1 TFA/methylene chloride) and allowed to stir at 25° C. for 1 hour. The product is washed with ether, washed with sodium bicarbonate, dried over sodium sulfate and condensed. Further purification can be achieved by standard chromatographic methodologies for small peptides.

### EXAMPLE 13

### A Soluble Combinatorial Library of Pyridyl Backbone Compositions

A soluble combinatorial library of pyridyl backbone compositions is useful for a wide range of medical, pharmacological, and scientific purposes. For example, a soluble combinatorial library of pyridyl backbone compositions is useful to the discovery of peptide analogues that display the same or enhanced biological activity as the activity of naturally occurring peptides.

### EXAMPLE 14

### Synthesis of the MeO-PEG-N_{[1]}-N_{[2]}-N_{[3]}-N_{[4]}-N_{[5]} Peptide Library

Validation of the LPCS method was achieved through the synthesis of a peptide library using our recursive deconvolution methodology. It will be recalled that the essence of recursive deconvolution is to build and hold a set of partially synthesized combinatorial libraries. The first LPCS library contained four components (Tyr, Gly, Phe, Leu) and five partial sub-libraries, to give a total library size was 1024. Because there were four components, four channels of synthesis were used, each involving the addition of a single component at any time.

Initiating the process required the splitting of MeO-PEG into four equal pools, in which Tyr, Gly, Phe, Leu are coupled to the homopolymer. Upon completion of each of the coupling reactions precipitation of the MeO-PEG-Naa (Naa=Tyr, Gly, Phe, Leu) was accomplished by the addition of diethyl ether. This allowed for the removal of excess coupling reagents by filtration of the MeO-PEG-Naa. The more polar contaminants were removed by simple recrystallization of the MeO-PEG coupled product. The importance of this step is that crystallization avoids the possibility of inclusions, which may occur with gelatinous precipitates. Additionally the excess of protected amino acids can be removed quantitatively. Portions of each of these sub-libraries are set aside and catalogued as partial libraries p(1). The remaining MeO-PEG-Naa is combined, solubilized and seperated into four portions, each channel is loaded, and Tyr, Gly, Phe, Leu are attached as before and polymer sublibraries are precipitated and crystallized. Again, an aliquot of this library is set aside as a partial library p(2), which now consists of four pools made up of MeO-PEG-N_{[1]}-Tyr, MeO-PEG-N_{[1]}-Gly, MeO-PEG-N_{[1]}-Phe, MeO-PEG-N_{[1]}-Leu. The remainder is again pooled and split, and the entire process is repeated for the assembly of sublibraries p(3), p(4) and a final sublibrary of p(5) {MeO-PEG-N_{[1]}-N_{[2]}-N_{[3]}-N_{[4]}-Tyr, MeO-PEG-N_{[1]}-N_{[2]}-N_{[3]}-N_{[4]}-Gly, MeO-PEG-N_{[1]}-N_{[2]}-N_{[3]}-N_{[4]}-Gly, MeO-PEG-N_{[1]}-N_{[2]}-N_{[3]}-N_{[4]}-Phe, MeO-PEG-N_{[1]}-N_{[2]}-N_{[3]}-N_{[4]}-Leu.

### EXAMPLE 15

### Recursive Deconvolution of the MeO-PEG-N_{[1]}-N_{[2]}-N_{[3]}-N_{[4]}-N_{[5]} Peptide Library: Screening for Anti-β-Endorphin Ligands

A competitive ELISA based methodology was devised, which when integrated into our recursive deconvolution strategy, allowed us to define the optimum ligands that inhibited the binding of leucine enkephalin (Tyr-Gly-Gly-Phe-Leu-OH) to anti-β-endorphin monoclonal antibody 3E7 (Meo, T., Gramsch, C. Inan, R., Hollt, V., Weber, E., Herz, A. & Reithmuller, G., Proc. Natl. Acad. Sci. USA 80:4084-4088, 1983.) This antibody binds to its natural epitope with high affinity (K_{d}=7.1 nM).

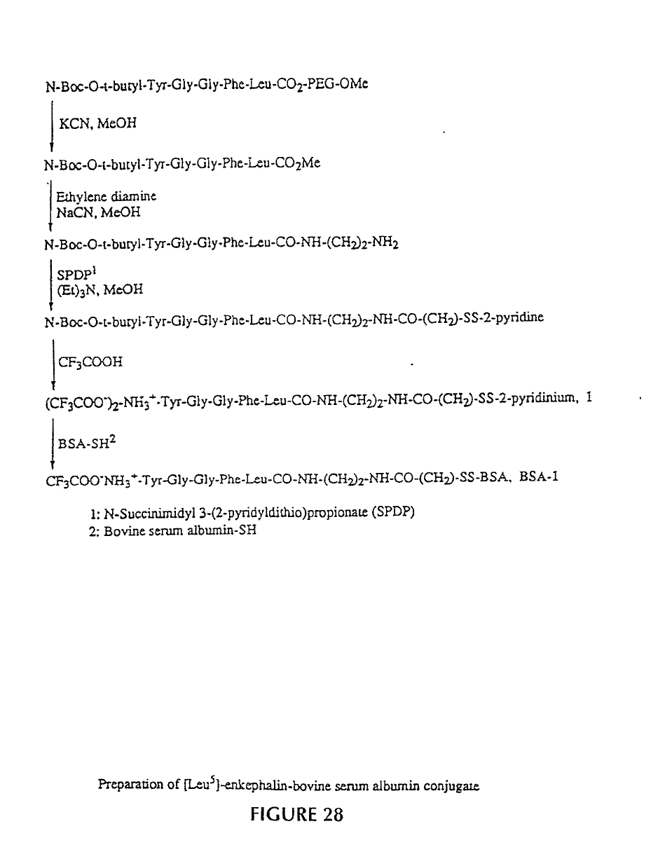

To set up the competition ELISA, attachment of the true ligand onto a protein of sufficient hydrophobicity had to be accomplished. A strategy was undertaken to chemically synthesize C-terminal pyridinium disulfide derivative 1, FIG. 28. By using this type of synthetic methodology the activated pentapeptide 1 was swiftly and cleanly coupled to bovine serum albumin (BSA) which had been modified with Traut's reagent. This BSA-1 conjugate thus provided a way to display the pentapeptide ligand on an ELISA plate. Some what cryptic but equally important was that this strategy also allowed the coupling process to be monitored since thiopyridine absorbs at 343 nm. This BSA-Tyr-Gly-Gly-Phe-Leu unit affixed to an ELISA plate allowed quantitation of Tyr-Gly-Gly-Phe-Leu or its analogs in solution by competition for binding of anti-β-endorphin to the immobilized Tyr-Gly-Gly-Phe-Leu. The amount of bound anti-β-endorphin could then be quantified by ELISA.

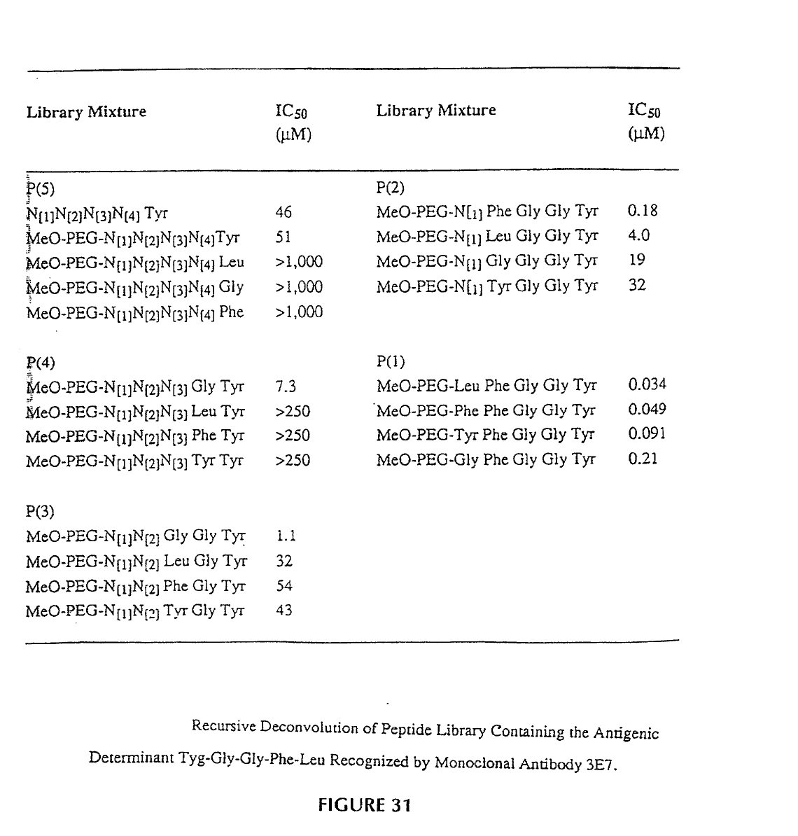

The diverse solubilizing power of MeO-PEG provided a direct way to screen the saved and catalogued MeO-PEG sublibraries in a homogeneous competition ELISA assay for binding to the β-endorphin antibody, FIG. 31. However, it should be noted that the library can be “deprotected” and the MeO-PEG removed to provide just the library of ligands. These sublibrary mixtures can also be searched in an analogous manner for prospective binding ligands, and as shown in FIG. 31, binding affinities detected are quite similar.

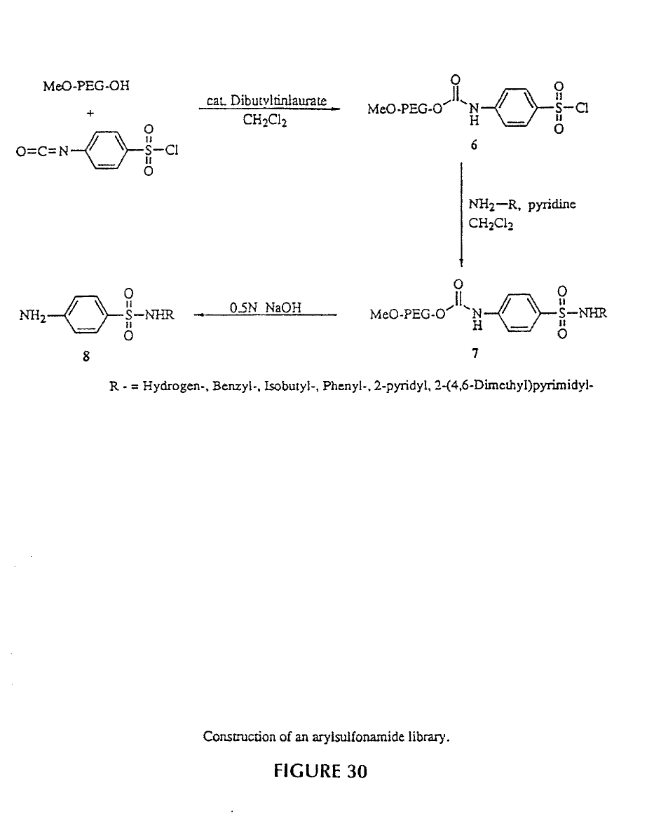

The deconvolution sequence can be followed by examining the IC₅₀ values determined for each p(n) sublibrary which is depicted in FIG. 31. Thus, starting with the four pools of the pentapeptide sublibrary p(5), where only the N-terminal amino acid is defined, the MeO-PEG-N_{[1]}-N_{[2]}-N_{[3]}-N_{[4]}-Tyr pool gave the only detectable binding IC₅₀=51 μM. Based on the recursive strategy, Tyr is coupled to the four saved and catalogued p(4) sublibraries giving MeO-PEG-N_{[1]}-N_{[2]}-N_{[3]}-Gly-Tyr, MeO-PEG-N_{[1]}-N_{[2]}-N_{[3]}-Phe-Tyr, MeO-PEG-N_{[1]}-N_{[2]}-N_{[3]}-Leu-Tyr, MeO-PEG-N_{[1]}-N_{[2]}-N_{[3]}-Tyr-Tyr. Assay of these four new pools provides an enrichment step and more importantly deconvolutes the next residue, glycine, (MeO-PEG-N_{[1]}-N_{[2]}-N_{[3]}-Gly-Tyr, IC₅₀=7.7 μM). These results allowed for a logical procession to the next saved sublibrary, p(3), wherein tyrosine and glycine are both coupled to the four p(3) pooled sequences. Solving for the third amino acid did not give a unique result but MeO-PEG-N_{[1]}-N_{[2]}-Gly-Gly-Tyr, the sequence corresponding to that of the native epitope, was the strongest binder IC₅₀=1.1 μM. The p(2) sublibrary was solved in a similar manner (vide infra), but now two pools, one containing the predicted sequence, (MeO-PEG-N_{[1]}-Phe-Gly-Gly-Tyr, IC₅₀=0.18 μM) and one containing the sequence (MeO-PEG-N_{[1]}-Leu-Gly-Gly-Tyr, IC₅₀=4.0 μM) were uncovered. At this point deduction of alternative active members could have been accomplished by tracing in succession both sequences Tyr-Gly-Gly-Phe and Tyr-Gly-Gly-Leu. However, because we have already examined this same pentapeptide library from a “solid phase” recursive deconvolution strategy we decided to only follow the most active component (Tyr-Gly-Gly-Phe) through the iterative process. In FIG. 30, the final p(1) sublibrary provided us with the native epitope and several other potent binders.

### EXAMPLE 16

### Liquid Phase Synthesis and Characterization of Non-Peptide, Nonoligomeric Molecules: Sulfonamides.

The LPCS process that has been described should allow for the synthesis of any class of molecular entity as long as the chemistry employed does not interact with, or adversely affect the polymer's properties. As a starting point for the examination of the MeO-PEG support under conditions other than peptide linking and deprotection reactions we investigated the polymer's potential in the context of synthesizing a class of compounds known as sulfonamides. Sulfonamides, because of their low cost and undeniable efficacy in susceptible infections, have for years spurred the preparation of numerous analogs. However, because of bacterial resistance, a relatively narrow antibacterial spectrum, and unacceptable side effects in some patients, the antibacterial sulfonamides no longer enjoy the clinical vogue they once had. Interestingly, because of these extensive clinical studies several pleasant surprises came out of this work. Namely a number of the arylsulfonamides which showed poor antibacterial potency now provided leads to new classes of drugs. These include new classes of endothelin antagonists, antitumor agents, and/or possess antiarrhythmic activity (31). The arylsulfonamide nucleus thus appears to be a significant pharmacophore on which to build a combinatorial library.

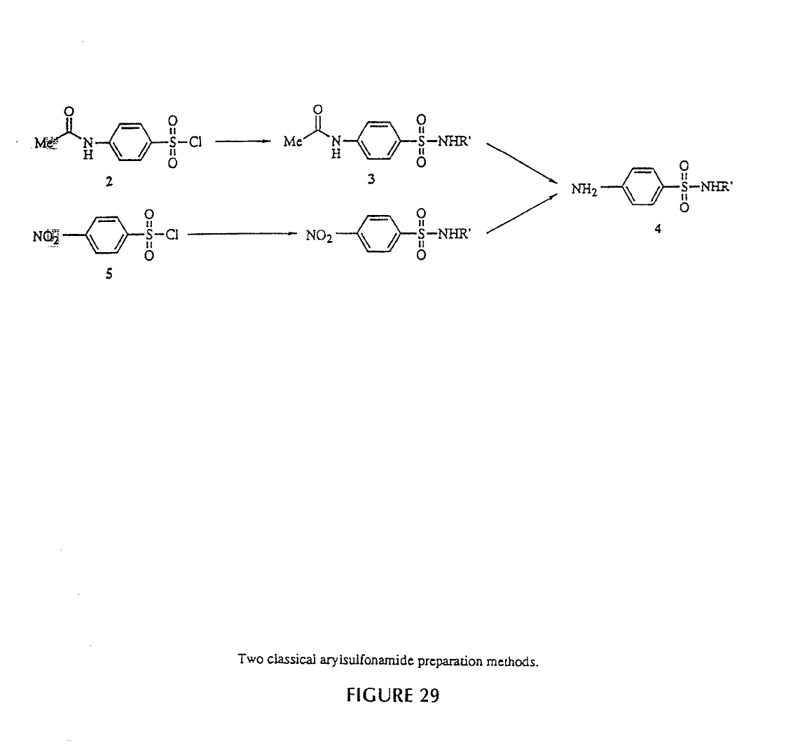

Before a library of any magnitude can be secured, a general synthetic scheme with reliable protocols for a variety of chemistries must be investigated. Past syntheses of arylsulfonamides that have lead to drugs, have been achieved by one of two fairly straightforward routes, (FIG. 29) (Ellingboe, J. W., Spinelli, W., Winldey, M. W., Nguyen, T. T., Parsons, R. W., Moubarak, I. F., Kitzen, J. M., Vonengen, D. & Bagli, J. F., J. Med. Chem. 35:707-716, 1992.) In the first methodology chlorosulfonation of acetanilide gives the corresponding sulfonyl chloride 2; and reaction with the appropriate amine gives the intermediate, 3. Hydrolysis in either acid or base leads to the sulfanilamide 4. In an alternative approach, the amide formation is performed on para-nitrobenzenesulfonyl chloride 5. Reduction by either chemical or catalytic methods directly affords the desired product. We envisioned an aryl sulfonyl chloride like 2 (FIG. 29) to be the key intermediate in our MeO-PEG synthesis and while both routes provide such an intermediate, neither present a convenient handle for the attachment of the aryl sulfonyl chloride appendage.

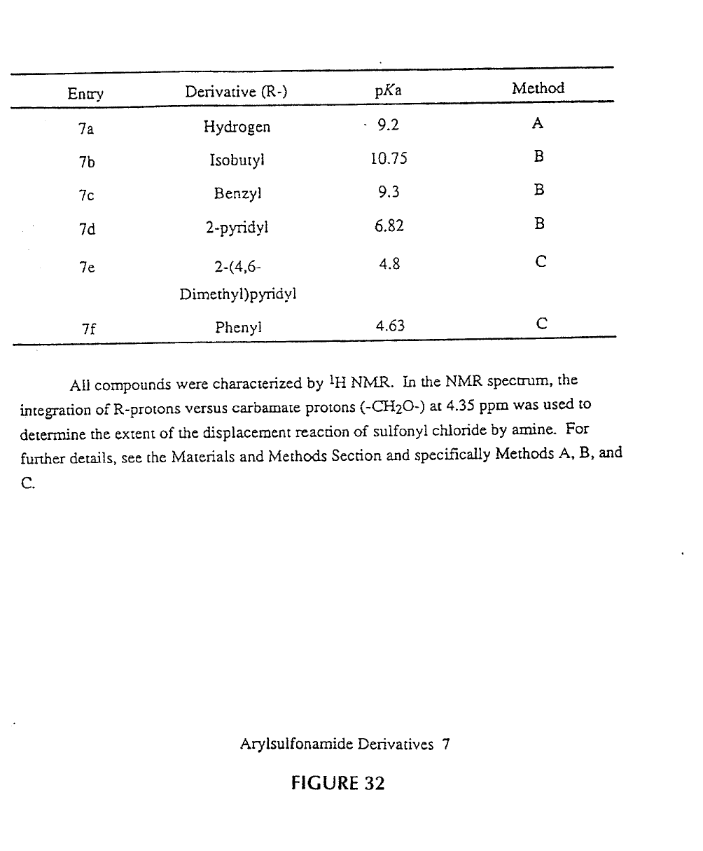

A new route (FIG. 30) was devised which provides the flexibility for added diversity and embraces in a simple manner the desired arylsulfonyl chloride. By starting with 4-(chlorosulfonyl)phenyl isocyanate the MeO-PEG support is functionalized and the desired sulfonyl chloride intermediated 6 is obtained in a single step. Most impressive is that there is no competing nucleophilic process at the chlorosulfonic acid moiety during this coupling reaction. Equally important is that this linkage allows the reaction to be followed by proton NMR (FIG. 32), is compatible with a variety of sulfonyl chloride nucleophilic addition reactions; yet at the end of the synthesis the carbamate which links the arylsulfonamide to the MeO-PEG is readily cleaved (NaOH) and the product isolated from homogeneous support. Employing the reaction scheme shown in FIG. 30 we have synthesized the structurally diverse, aryl sulfonamides 8 in multimilligram quantity (FIG. 30 and FIG. 32). It should be noted that while the key intermediate is sulfonyl chloride 6, the overall success of the aryl sulfonamides synthesized, as shown in FIG. 32, is highly dependent on the pKa of the nucleophile. Therefore very poor nucleophiles like 7e and 7f require longer reaction times and more stringent temperatures (FIG. 32).

Peptide libraries secured on solid supports were the first chemically synthesized combinatorial libraries (Geysen, H. M., Rodda, S. J. & Mason, T. J., Mol. Immunol. 23:709-715, 1986; Lam, K. S., Salmon, S. E., Hersch, E. M., Hruby, V. J., Kazmierski, W. M. & Knapp, R. J., Nature (London) 354:82-84, 1991; Houghton, R. A., Pinilla, C., Blondelle, S. E., Appeal, J. R., Dooley, C. T. & Curevo, J. H. Nature (London) 354:84-86, 1991; Foder, S. P. A., Read, J. L., Pirrung, M. C., Styer, L., Lu, A. T. & Solas, D., Science 251:767-773, 1991.) As important as this work was, the need for greater chemical diversity was quickly recognized and an explosion of non-oligomeric heterocyclic libraries has begun to dominate the combinatorial scene (Bunnin, B. A., Plunkett, M. J. & Ellman, J. A., Proc. Natl. Acad. Sci. USA 91:4708-4712, 1994; Gordon, D. W. & Steele, J., Bioorg. Med. Chem. Lett. 5:47-50, 1995; Pirrung, M. C. & Chen, J., J. Am. Chem. Soc. 117:1240-1245, 1995; Willard, R., Jammalamadaka, V., Zava, D., Hunt, C. A., Benz, C. C., Kushner, P. J. & Scanlan, T. S., Chem. Biol. 2:45-51, 1995.) An outgrowth of these classes of libraries is the fervent pace to try and adapt solid phase synthesis to multistep organic reaction sequences. We have proposed and implemented a technology termed Liquid Phase Combinatorial Synthesis to simplify and thus further accelerate this process. In this methodology we exploit the advantages that classic organic synthesis offers in solution with those that solid phase synthesis can provide.

The results reported in this article indicate that the reaction scope of LPCS should be general. Its value to multi-high-throughput screening assays could be of particular merit since multi-milligram quantities of each library member can be attained. The principles and methods outlined using LPCS should be applicable to the synthesis of complex chemical structure libraries as well as other processes that fall under the heading of chemical diversity.

Materials and Methods

General.

BOC-protected amino acids were purchased from Bachem California. N-succinimidyl-3-(2-pyridyldithio) propionate (SPDP) was purchased from ProChem. All other reagents including poly(ethylene glycol) methyl ether (M.W. 5000) were purchased from Aldrich. Methylene chloride and chloroform were purified by distilling over CaH₂, and methyl alcohol was distilled over magnesium turnings. N,N-dimethylformamide was successively dehydrated over oven-dried molecular sieves (4A). Other solvents were used as commercially available, or otherwise mentioned. TLC eluent was CHCl₃:MeOH:AcOH:H₂O=83:15:1:1. UV spectra were measured on a Hewlett-Packard 8452A diode array spectrophotometer at ambient temperature.

Construction of the Pentapeptide Library.

The pentapeptide libraries were constructed manually on monomethoxy polyethyleneglycol (MeO-PEG) polymer support by split-synthesis (13) and Bayer's protocol (7) with the following modifications. N-BOC-L-Leu, N-BOC-Gly, N-BOC-L-Phe, and N-BOC-O-(2-Br-Cbz)-Tyr were amino acid components for the library construction. The first amino acid residue was anchored to the MeO-PEG by the DCC/DMAP coupling method (Zalipsky, S., Gilon, C. & Zilka, A., J. Macromol. Sci. Chem. A21:839-834, 1984). The coupling efficiency was determined to be >99%, based on the absorbance of phenyl carbamate derivative (ε236 nm=17,500 M⁻¹cm⁻¹) which was quantitatively formed by the reaction between the unreacted hydroxyl groups of MeO-PEG and phenyl isocyanate in the presence of a catalytic amount of dibutyltinlaurate. The next amino acids were added sequentially with the aid of O-benzotriazol-1Y-L-N,N,N′,N′-Tetramethyluronium Hexafluorophosphate (HBTU) and diisopropylethylamine (DIPEA) (Dourtoglou, V., Gross, B., Lambropoulou, V., & Zioudrou, C., Synthesis A:572-574, 1984). Each coupling reaction was run in a mixed solvent of CH₂Cl₂ and DMF until Kaiser's ninhydrin test (Kaiser, E., Colescott, R. L., Bossinger, C. D. & Cook, P. I., Anal. Biochem. 34:595-598, 1979) was negative; acetic anhydride was used to cap any uncoupled amino groups. After each coupling step, a portion of the polymer was saved and labelled for future use according to the recursive deconvolution method of combinatorial chemical libraries (12). The final deprotection of N-Boc- and O-(2-Br-Cbz)-groups by iodotrimethylsilane (Lott, R. S., Chauhan, V. & Stammer, C. H., J. Chem. Soc. Chem. Commun. 495-496, 1979) completed the construction of the pentapeptide libraries.

Preparation of [Leu⁵]-enkephalin-bovine Serum Albumin Conjugate (BSA-1).

[Leu⁵]-Enkephalin was coupled to bovine serum albumin making BSA-1. The scheme used to prepare BSA-1 is shown in FIG. 28. It should be noted that the coupling of 1 to BSA requires the reformulation of BSA to a sulfhydrylated protein by Trauts reagent.

N-Boc-O-t-Butyl-Tyr-Gly-Gly-Phe-Leu-CO₂-PEG-OMe.

MeO-PEG (5 g, 1 mmol), N-Boc-Leu H₂O (0.748 g, 3 mmol), and DMAP (0.0306 g, 0.25 mmol) were dissolved in methylene chloride (25 mL) and DCC (1.24 g, 6 mmol) was added. After 2 hrs of stirring at room temperature, acetic anhydride (1 mL) was added, and stirring was continued for another 30 min. Urea was filtered off, and to the filtrate was slowly added ethyl ether with vigorous stirring. The precipitate was collected on a glass filter and then redissolved in DMF. The compound was reprecipitated by addition of ethyl ether and the precipitate washed with ethanol to produce pure N-BOC-Leu-CO₂-PEG-OMe (I) (5.15 g, 99%) . I (5.15 g) was dissolved in a mixture of methylene chloride: trifluoroacetic acid (1:1, 40 mL) and stirred for 30 min. at room temperature. The volume of solvent was reduced to half, and the slow addition of ethyl ether gave the ammonium trifluoroacetate salt (II) as a white precipitate (4.98 g, 96%) . II (4 g, 0.765 mmol), N-Boc-Phe (0.609 g, 2.30 mmol), and DIPEA (1.3 mmol, 7.65 mmol) were dissolved in a mixture of methylene chloride and DMF (25 mL), followed by the addition of HBTU (0.871 g, 2.30 mmol). The reaction was monitored by Kaiser's ninhydrin test untill a negative reading was obtained. Acetic anhydride (1 mL) was then added and stirring was continued for another 30 min. The reaction mixture was condensed to half volume. Successive operations of precipitation by ethyl ether, redissolution in DMF, reprecipitation by ether, and a final wash of the precipitate by ethanol generated N-BOC-Phe-Leu-CO₂-PEG-OMe (III) (3.91 g, 95%). The deprotection of N-BOC group by a TFA:methylene chloride mixture gave an ammonium trifluoroacetate salt (IV) as a white precipitate (3.75 g, 96%). The repetitions of a cycle of coupling and deprotection with N-BOC-Gly, N-BOC-Phe, and N-BOC-O-t-Butyl-Tyr produced N-Boc-O-t-Butyl-Tyr-Gly-Gly-Phe-Leu-CO₂-PEG-OMe (2.51 g, 96%).

N-Boc-O-t-Butyl-Tyr-Gly-Gly-Phe-Leu-CO₂Me.

N-Boc-O-t-Butyl-Tyr-Gly-Gly-Phe-Leu-CO₂-PEG-OMe (2 g, 0.35 mmol) and KCN (200 mg, 3.08 mmol) were dissolved in MeOH (10 mL), and stirred at room temperature until N-Boc-O-t-Butyl-Tyr-Gly-Gly-Phe-Leu-CO₂-PEG-OMe disappeared as monitored by TLC (24 hrs). The reaction mixture was concentrated to 3 mL, acidified with 1N HCl, and extracted with EtOAc twice. The combined ethyl acetate layer was washed with brine and dried over MgSO₄. The solvent was removed under reduced pressure to give the desired product (0.273 g, 93%) TLC R_{f} 0.61; Electrospray-MS m/z 726 (M+H⁺) 748 (M+Na⁺).

N-Boc-O-t-Butyl-Tyr-Gly-Gly-Phe-Leu-(C═O)—NH—(CH₂)₂—NH₂.

The peptide methyl ester (80 mg, 0.11 mmol) NaCN (20 mg, 0.41 mmol), and ethylene diamine (400 μL, 5.99 mmol) were dissolved in MeOH. The resulting mixture was heated at 45° C. for 8 hrs. The reaction mixture was cooled, concentrated, and acidified with 1N HCl. This was partitioned between EtOAc and aqueous CuSO₄ and the organic layer was successively washed with aqueous CuSO₄ until ethylene diamine could not be detected in the ethyl acetate solution. The ethyl acetate solution was dried over MgSO₄, and removal of the solvent gave the desired product (62 mg, 75%). TLC R_{f} 0.15: Electrospray-MS m/z 754 (M+H⁺).

N-Boc-O-t-Butyl-Tyr-Gly-Gly-Phe-Leu-(C═O)—NH—(CH₂)₂—NH—(C═O)—(CH₂)₂—SS-2-pyridine.

The peptide amide (9.2 mg, 12 umol) and N-Succinimidyl 3-(2-pyridyldithio)propionate (3.8 mg, 12 μmol)(SPDP) were dissolved in MeOH (5 mL). Two drops of triethylamine were added and the reaction mixture was stirred for 1 hr at room temperature. The reaction was evaporated to dryness, and purified by the preparative TLC (10.7 mg, 92%). TLC R_{f} 0.55; FAB-MS m/z 951 (M+H⁺), 973 (M+Na⁺).

(CF₃COO⁻)—NH₃₊-Tyr-Gly-Gly-Phe-Leu-(C═O)—NH—(CH₂)₂—NH—(C═O)—(CH₂)₂—SS-2-pyridinium.

The N-BOC and O-t-butyl groups were deprotected by the stirring of the above N-hydroxysuccinimide ester (10.7 mg, 11.3 μmol) in trifluoroacetic acid (2 ml) for 17 hrs. All volatiles were removed and upon addition of ethyl ether, the desired product was formed as a tan solid (11 mg, 95%). FAB-MS m/z 795 (M+H⁺), 817 (M+Na⁺)

[Leu⁵]-Enkephalin-bovine Serum Albumin Conjugate.

The above salt (2 mg, 1.96 μmol) was dissolved in DMF (50 μL) and the solution slowly added to the bovine serum albumin (sulfhydrylated by Traut's reagent) in PBS (1 mL). After 10 min., an aliquot of 50 μL was removed and diluted to 1 mL. The concentration of [Leu⁵]-enkephalin-bovine serum albumin conjugate was determined to be 0.8 mM based on the formation of 2-thiopyridine (A₃₄₄=0.2529). The pyridine-2-thione has a molar extinction coefficient of 8080 M⁻¹cm⁻¹ at 343 nm.

Partial Library Competition ELISA for Anti-β-Endorphin Monoclonal Antibody.

Each well of a Costar 96-well microtitre plate was initially coated with 25 μL of BSA-1 (5-20 mg/mL) in 60 mM sodium bicarbonate/30 mM sodium carbonate, (pH 9.3) overnight. The wells were washed with deionized water and blocked 100 μL of BLOTTO to prevent nonspecific adsorption. After incubating for ½ hour at 37° C. in a moist chamber, the BLOTTO was shaken out and 25 μL of the partial library pool (competing antigen) was added to the first well and serially diluted across the plate; the same process was then continued in the first well of the second row. Lane 12 was used as the positive control (It should be noted that this serial dilution step was used for all competing partial library pools). The anti-β-endorphin antibody was added to each well (25 μL) and the plate incubated at 37° C. for 2 h. The plate was washed twenty times with deionized water, and 25 μL of a 1:1000 dilution of goat anti-mouse IgG glucose-oxidase conjugate (Cappel) was added to each well and the plate incubated at 37° C. for 1 hour. The plates were washed twenty times with deionized water, and bound antibody detected by the addition of 50 μL of developing agent (0.6 μL 20% glucose, 40 μL ABTS, 40 μL HRPO in 5 mL of phosphate buffer pH 6.0) to each well. Thirty minutes later the plates were read at 405 nm.

Construction of a Sulfonamide Library.

The arylsulfonamide library was constructed on the MeO-PEG support by parallel synthesis. MeO-PEG was reacted with 4-(chlorosulfonyl)phenyl isocyanate in the presence of catalytic amount of dibutyltinlaurate to give 4-(chlorosulfonyl)phenyl carbamate of MeO-PEG (6). Compound 6 was divided into 6 portions, and the reaction of each portion with 6 different amines in the presence of pyridine generated sulfonamides 7. The basic hydrolysis of these MeO-PEG sulfonamides completed the construction of an arylsulfonamide library consisting of 6 members (FIG. 30).

O-(MeO-PEG)-N-[(4-chlorosulfonyl)phenyl]carbamate.

4-(Chlorosulfonyl)phenyl isocyanate (0.653 g, 3 mmol) was added to MeO-PEG (5 g, 1 mmol) in methylene chloride (50 mL) and two drops of dibutyltinlaurate added (Bayer, E., Gatfield, I., Mutter, H. & Mutter, M., Tetrahedron 34:1829-1831, 1978). After 5 hrs of stirring at room temperature, ethyl ether was slowly added to the vigorously stirred reaction mixture. The precipitate was collected on a glass filter and thoroughly washed with ethyl ether. The precipitate was dried under vacuum to yield the desired product quantitatively.

N-(4-Alkylminosulfonyl)phenyl-O-(MeO-PEG) Carbamate.

N-(4-Alkylminosulfonyl)phenyl-O-MeO-PEG carbamate was prepared i) by continuously bubbling ammonia gas through O-(MeO-PEG)-N-[(4-chlorosulfonyl)phenyl]carbamate (0.5 g, 95.8 μmol) in methylene chloride (5 mL) containing pyridine (20 eqs.) for 24 hrs at room temperature (Method A), by stirring O-(MeO-PEG)-N-[(4-chlorosulfonyl)phenyl]carbamate (0.5 g, 95.8 μmol) with an excess of amine (15 eqs) in methylene chloride (5 mL) containing pyridine (20 eqs.) for 24 hrs at room temperature (Method B) (Winterbottom, R., J. Am. Chem. Soc. 62:160-161, 1940), or by heating the reaction mixture in pyridine solvent at 65° C. for an hour (Caldwell, W. T., Kornfeld, E. C., & Donnell, C. K., J. Am. Chem. Soc. 63:2188-2190, 1941). The MeO-PEG polymer was precipitated from the homogeneous solution by the addition of ethyl ether washed with with ethanol, and dried under vacuum to give the desired product quantitatively.

Sulfonamide.

N-(4-Alkylaminosulfonyl)phenyl-O-(MeO-PEG) carbamate (0.45 g) was dissolved in 0.5N NaOH (10 mL), and heated at 90° C. for 30 min (Winterbottom, R., J. Am. Chem. Soc. 62:160-161, 1940; Caldwell, W. T., Kornfeld, E. C. & Donnell, C. K., J. Am. Chem. Soc. 63:2188-2190, 1941). The reaction mixture was cooled to 4° C., and neutralized to pH 6-8 with concentrated HCl. The reaction mixture was extracted with ethyl acetate three times and the combined ethyl acetate layer was washed with brine, and dried over MgSO₄. The removal of solvent gave an analytically pure product (based on NMR spectrum). The reaction yield was typically 95-97%.

A new concept termed Liquid Phase Combinatorial Synthesis (LPCS) is described. The central feature of this methodology is that it exploits the advantages that classic organic synthesis in solution offers with those that solid phase synthesis can provide, through the application of a linear homogeneous polymer. To validate this concept two libraries were prepared one of peptide and the second of non-peptide origin. The peptide based library was synthesized using a recursive deconvolution strategy, (Erb, E., Janda, K. D., Brenner, S., Proc. Natl. Acad. Sci. USA 91:11422-11426, 1994) and several ligands were found within this library to bind a monoclonal antibody elicited against β-endorphin. The non-peptide molecules synthesized were arylsulfonamides, this class of compounds were conscripted based on their known clinical bactericidal efficacy, (Maren, T. H., Annu. Rev. Pharmacol. Toxicol. 16:309-327, 1976). The results reported in this article indicate that the reaction scope of LPCS should be general, while its value to multiple, high-throughput screening assays could be of particular merit, since multi-milligram quantities of each library member can readily be attained.

### Synthetic Methods

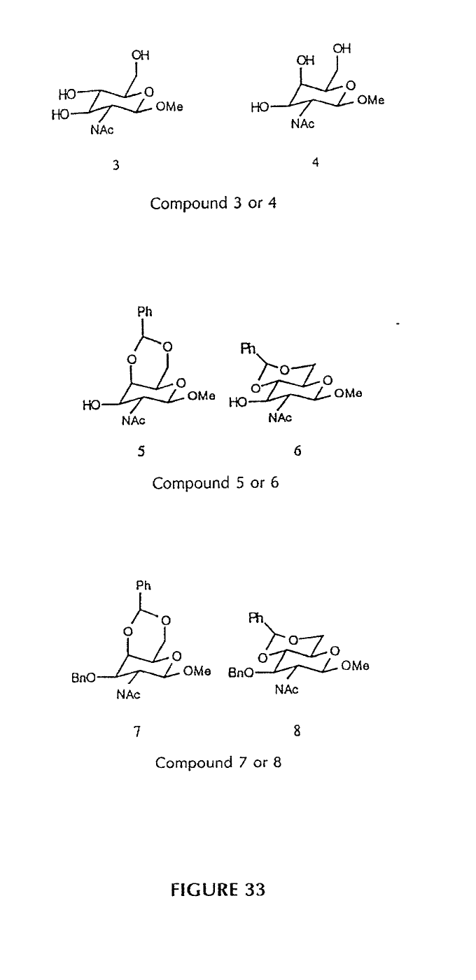

Preparation of Compound 3 or 4 (Structure of Compounds 3 and 4 is Depicted on FIG. 33).

Procedure as described in Methods in Carbohydrate chemistry, Whistler, R., II, 1963, p. 327. A mixture of 80 grams (g) anhydrous D-glucosamine hydrochloride or D-galactosamine hydrochloride from Aldrich chemical company, in 200 milliliter (mL) methanol and 20 g Dowex 50 (H⁺) acidic resin, is stirred at the boiling point in a round bottom flask. After a 24-hour reaction time, the resin is removed by filtration and washed three times with 20 ml. of methanol. The filtrate and washings are combined and concentrated to about 125 ml by rotovap. The concentrate is allowed to cool to room temperature and purification by crystallization or by flash column chromatography yields 3 or 4 and carry on as follows.

A solution of crude (1.0 equiv.) in CH₂Cl₂ (0.4 M) is cooled to 0° C. The solution is next treated with 4-DMAP (0.2 equiv.), triethylamine (8.0 equiv.) and dropwise addition of acetic anhydride (365 mL, 3.84 mol, 7.0 equiv.). The reaction is stirred for 1 h at 0° C. and then quenched upon dropwise addition of 5% HCl at 0° C. or until neutral pH. Next, the reaction is diluted with ether and then washed with saturated aqueous NaHCO₃ (2×), water (1×) and brine (1×). The aqueous layer is back extracted with ether (1×) and then recombined with the original organic layer, which is dried (MgSO₄) and then evaporated.

A solution of the crude (1.0 equiv.) in methanol (0.5 M), is treated with NaOMe (0.1 equiv.) and allowed to stir at 25° C. for 24 h. The reaction mixture is then condensed and purified by flash column chromatography or crystallized and yields compound 3 or 4.

Preparation of Compound 5 or 6 (Structure of Compounds 5 and 6 is Depicted on FIG. 33).

To a solution of 3 or 4, in methylene chloride (0.5 M), is added benzaldehyde dimethyl acetal, purchased from Aldrich company (1.2 equiv.), ZnCl₂ (0.1 equiv.) and the reaction mixture is stirred for overnight at 25° C. The product 5 or 6 is then crystallized or purified by flash column chromatography and carried onto the next step.

Preparation of Compound 7 or 8 (Structure of Compounds 7 and 8 is Depicted on FIG. 33).

To a solution of alcohol 5 or 6 (1.0 equiv.) in DMF (0.5 M) at 0° C., is added KH (1.1 equiv., 35% dispersion in mineral oil) over several portions. The reaction mixture is warmed to room temperature and stirred 1 h. Next, the reaction is cooled to 0° C. and treated with benzyl bromide (1.1 equiv.) and stirred for 1.5 h. A saturated solution of ammonium chloride is added dropwise to quench the reaction mixture at 0° C. and the mixture is diluted with ethyl acetate (2L), washed with water (2×100 mL), brine (1×100 mL), dried over MgSO₄ and evaporated. Purification by crystallization or by flash column chromatography yields 7 or 8.

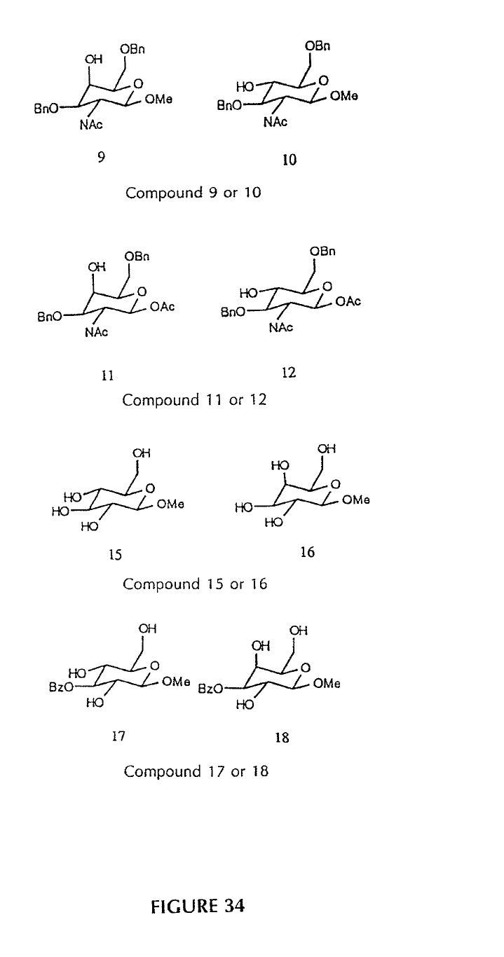

Preparation of Compound 9 or 10 (Structure of Compounds 9 and 10 is Depicted on FIG. 34).

Conditions as described by Johansson, R., Samuelsson, B., J. Chem. Soc., Chem. Commun., 201, 1984. To a solution of 7 or 8 (1.0 equiv.) and sodium cyanoborohydride (5.0 equiv.) in dimethylformamide (DMF) (0.1 M) containing powdered 3 Å molecular sieves at 0° C., is added trifluoroacetic acid (10 equiv.) dissolved in DMF (1.0 M). When t.l.c. indicates complete reaction, the product is purified by flash column chromatography or crystallization and yields the desired 9 or 10.

Preparation of Compound 11 or 12 (Structure of Compounds 11 and 12 is Depicted on FIG. 34).

To a solution of the intermediate 9 or 10 (1.0 equivalents) in methylene chloride (0.10 Molar), is dissolved 2,6 lutidine (1.3 equivalents) at 0° C. Subsequent addition of triisopropyl or triethylsilyl trifluoromethane-sulfonate (1.3 equivalents) is followed by stirring for 2 hours and then the reaction is diluted with diethylether and washed with ammonium chloride (2×), brine (1×) and then purified by crystallization. Compound is carried on as follows.

The intermediate is then exposed to a mixture of acetic anhydride (1.1 equiv.) in acetic acid (0.5 M) and stirred at 0° C. to 60° C. for 1 hour. After completion of reaction, the mixture is diluted with methylene chloride, neutralized with NaHCO₃ and then washed with water (1×) and brine. The compound is then precipitated from ether and recrystallized from ethanol and carried on as follows.

The intermediate is exposed to a mixture of tetrabutylammoniumfluoride (1.5 equivalents) from Aldrich company in methylene chloride (0.1 Molar) and stirred for 1 hour at 0° C. The compound is then is diluted with methylene chloride, neutralized with NaHCO₃ and then washed with water (1×) and brine. The compound is then precipitated from ether and recrystallized from ethanol and affords the compound 11 or 12.

Preparation of Compound 15 or 16 (Structure of Compounds 15 and 16 is Depicted on FIG. 34).

Procedure as described in Methods in Carbohydrate chemistry, Whistler, R., II, 1963, p. 327. A mixture of 80 g anhydrous D-glucose or D-galactose from Aldrich chemical company, in 200 mL. methanol and 20 g Dowex 50 (H⁺) acidic resin, is stirred at the boiling point in a round bottom flask. After 24-hr. reaction time, the resin is removed by filtration and washed three times with 20 ml. of methanol. The filtrate and washings are combined and concentrated to about 125 ml by rotovap. The concentrate is allowed to cool to room temperature and the product crystallizes overnight.

Preparation of Compound 17 or 18 (Structure of Compounds 17 and 18 is Depicted on FIG. 34).

Tetrol 15 or 16 (1.0 equiv.) is azeotroped with benzene (2×100 mL) and then dried overnight under vacuum over P₂O₅. A mixture of triol, dibutyl tin oxide (1.2 equiv.) and dry methanol (0.25 M) are heated at reflux for 4 h until the solution became clear and homogeneous. (An automatic stirring apparatus may be necessary.) The solvent is next removed in vacuo to give a foamy white tin complex which is then azeotroped with benzene (2×100 mL) and dried (2 h to overnight) under vacuum over P₂O₅. Next, anhydrous DMF (2.18 L, 0.2M) is added to redissolve the tin complex and then CsF (1.2 equiv.) and finally Benzyl bromide (0.5 equiv.) are added and then heated (40° C.) overnight. The clear solution is partially distilled under vacuum, (3.3 mm Hg, 75-100° C.) to obtain ⅕ the original volume of solvent. Reaction mixture is then diluted with ethyl acetate (2L) and washed with a small amount of water (2×100 mL) to remove cesium salts. Aqueous layer is back extracted with ethyl acetate (3×500 mL) and then recombined with the organic layer which is then dried over MgSO₄ and evaporated. Purification by flash column chromatography or crystallization affords the desired triol 17 or 18.

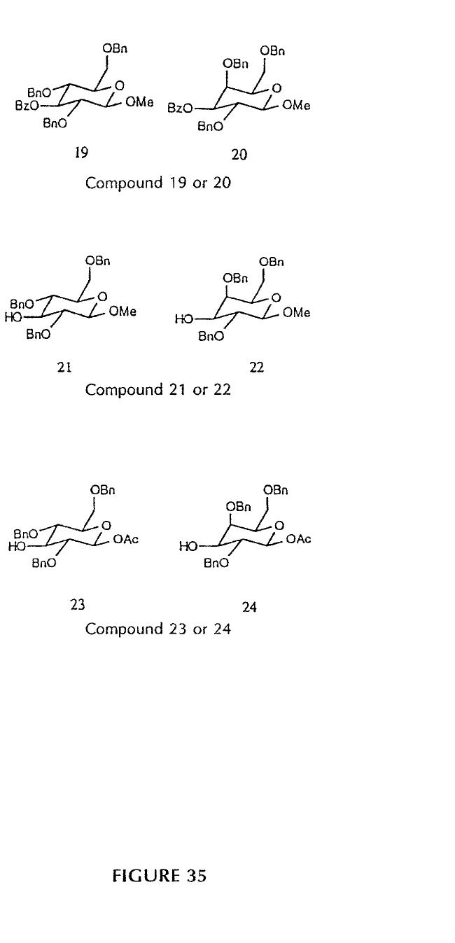

Preparation of Compound 19 or 20 (Structure of Compounds 19 and 20 is Depicted on FIG. 35).

To a solution of triol 17 or 18 (1.0 equiv.) in DMF (0.25 M) at 0° C., is added KH (3.3 equiv., 35% dispersion in mineral oil) over several portions. The reaction mixture is warmed to room temperature and stirred 1 h. Next, the reaction is cooled to 0° C. and treated with benzyl bromide (3.3 equiv.) and stirred for 1.5 h. A saturated solution of ammonium chloride is added dropwise to quench the reaction mixture at 0° C. and the mixture is diluted with ethyl acetate (2L), washed with water (2×100 mL), brine (1×100 mL), dried over MgSO₄ and evaporated. Purification by crystallization or by flash column chromatography yields 19 or 20.

Preparation of Compound 21 or 22 (Structure of Compounds 21 and 22 is Depicted on FIG. 35).

To a solution of benzoate 19 or 20 (1.0 equiv.) is added THF (0.464 M) and the solution is cooled to 0° C. The reaction mixture is then exposed to a 1.0 M solution of DIBAL (1.1 equiv.) and allowed to stir for 2.5 hour. The solution is then diluted with ether (5 mL) and then 2 mL of Rochelle salts (NaK—tartrate saturated solution) are added and the mixture is stirred for and additional 1 hour at 25° C. The reaction is then washed with water (2×10 mL), brine (1×5 mL) and then dried over MgSO₄. The compound is purified by flash column chromatography or crystallization.

Preparation of Compound 23 or 24 (Structure of Compounds 23 and 24 is Depicted on FIG. 35).

To a solution of the intermediate 21 or 22 (1.0 equivalents) in methylene chloride (0.10 Molar), is dissolved 2,6 lutidine (1.3 equivalents) at 0° C. Subsequent addition of triisopropyl or triethylsilyl trifluoromethane-sulfonate (1.3 equivalents) is followed by stirring for 2 hours and then the reaction is diluted with diethylether and washed with ammonium chloride (2×), brine (1×) and then purified by crystallization. Compound is carried on as follows.

The intermediate is then exposed to a mixture of acetic anhydride (1.1 equiv.) in acetic acid (0.5 M) and stirred at 0° C. for 1 hour. After completion of reaction, the mixture is diluted with methylene chloride, neutralized with NaHCO₃ and then washed with water (1×) and brine. The compound is then precipitated from ether and recrystallized from ethanol and carried on as follows.

The intermediate is exposed to a mixture of tetrabutylammoniumfluoride (1.5 equivalents) from Aldrich company in methylene chloride (0.1 Molar) and stirred for 1 hour at 0° C. The compound is then is diluted with methylene chloride, neutralized with NaHCO₃ and then washed with water (1×) and brine. The compound is then precipitated from ether and recrystallized from ethanol and yields the compound 23 or 24.

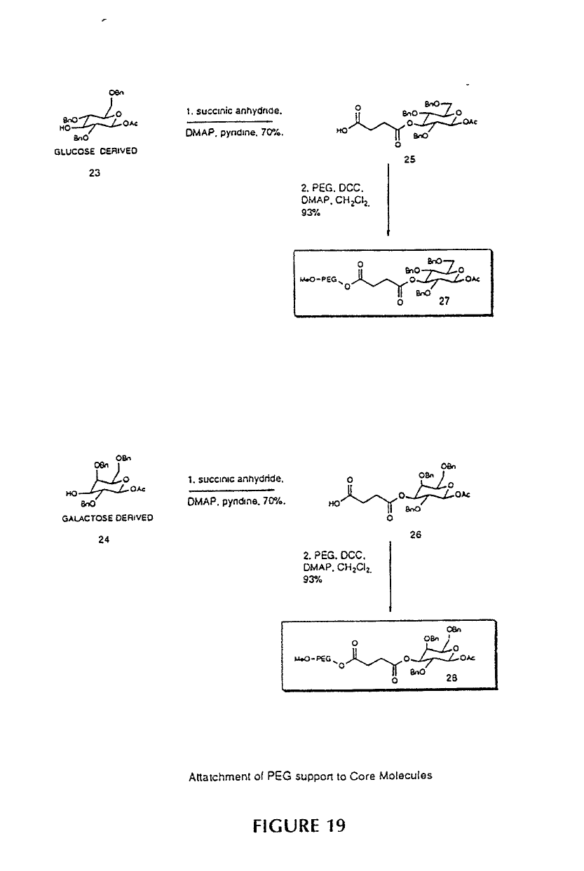

Preparation of Compounds 27 or 28 (Structure of Compounds 27 and 28 is Depicted on FIG. 35; See also FIG. 19).

The C-4 differentiated carbohydrates 23 or 24 are coupled to PEG (preferably the monomethylether, 5000 M.W., from Aldrich, Fluka or Sigma chemical companies) via an ester/succinyl linkage (1.5 equiv. succinyl chloride, 1.1 equiv. DMAP-dimethylaminopyridine, 0.5 M pyridine), although other functionalities such as ether and amide linkages are suitable as the linker between the PEG soluble support and the nucleotide core molecules. PEG succinate coupling to compounds 25 or 26, is accomplished via standard activated ester procedures employing DCC/DMAP conditions as developed by Keck et al., J. Org. Chem. 50:2394, 1985. For a standard protocol linking PEG to a Boc-Gly-OH residue, see Bayer et al., The Peptides, 2:309, Academic Press, 1979. The desired compounds 27 or 28 are then purified by precipitation and recrystallization from ether and ethanol.

A typical procedure is as follows: protected sugar 23 or 24, succinic anhydride (5 equiv.) and DMAP (1 equiv) are stirred in dry pyridine (0.20 M) at room temperature. After completion of the reaction, pyridine is removed by evaporation and the residue is subjected to flash chromatography in ethyl acetate. Next, the monomethylether of PEG (0.8 eq.) is mixed with the 3-O-hemisuccinate 25 or 26 and dried overnight at high vacuum over the drying agent phosphorous pentoxide P₂O₅. This mixture is then dissolved in anhydrous methylene chloride (0.5 M) and a catalytic amount of dimethylaminopyridine—DMAP (0.1 equiv.) is followed by dicylcohexylcarbodiimide—DCC (0.8 equiv.) After 15 minutes, the reaction becomes cloudy and is stirred overnight at room temperature. The precipitated urea is removed by filtration, washed with methylene chloride and the volume of the combined filtrates is reduced to its original size. It is cooled to 0° C., anhydrous ether is added with vigorous stirring, and precipitated out. After filtration the solid is dissolved in hot absolute ethanol, and recrystallized to afford 27 or 28 (Procedure from Krepinsky et al., J. Am. Chem. Soc. 113:5095, 1991, supplementary materials).

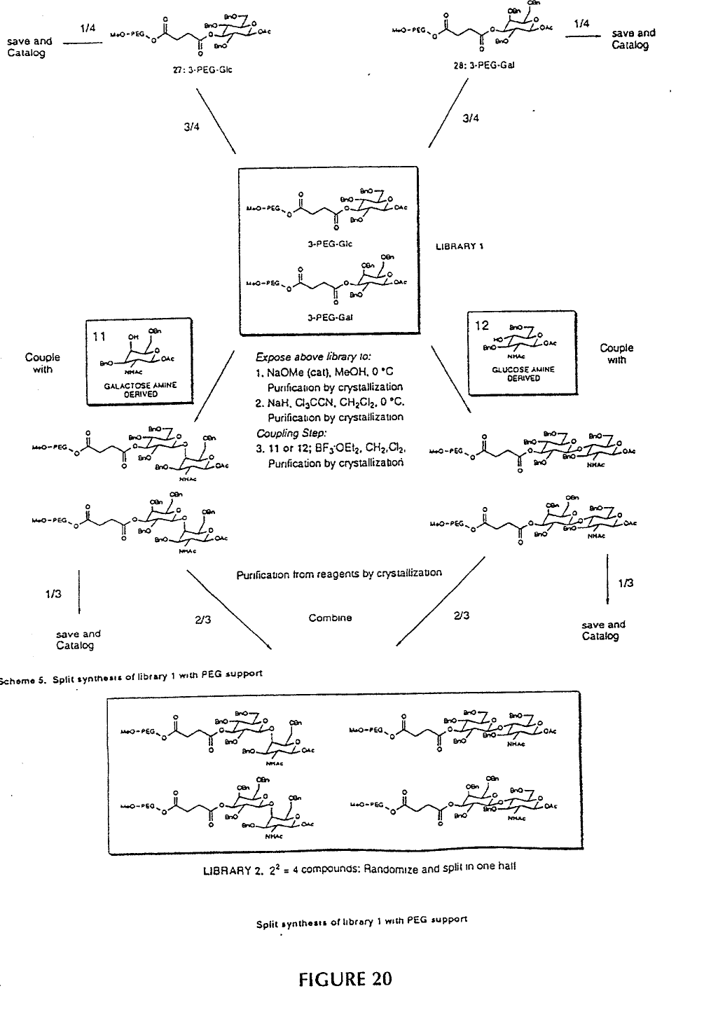

Preparation of Carbohydrate Library 2 (See FIG. 20)

Step 1. To a solution of library 1 in MeOH (0.10 M), is added a catalytic amount of NaOCH₃ (0.10 equivalents) and the mixture is stirred for 1 hour at 0° C. or until lactol formation is complete by thin layer chromatography. Reaction mixture is then quenched with ammonium chloride (1.0 M) and then diluted with ethyl acetate (0.05 M) and washed with water (1×), brine (1×) and condensed. Crude compound is then precipitated with ethyl ether and filtered. The PEG conjugate is allowed to crystallize out from hot ethanol, filtered and washed with cold ethanol to afford the desired intermediate lactol library.

Step 2. The intermediate lactol library is then converted to the acetimidate in a solution of methylene chloride (0.10 Molar) at 0° C. The mixture is next exposed to sodium hydride (1.2 equivalents) and allowed to stir for 1 hour and then trichloroacetonitrile (1.2 equivalents) is added in one step. After an additional hour (or until the reaction appears complete i.e. thin layer chromatography), the reaction mixture is quenched with a saturated solution of sodium bicarbonate (1.0 M) and then diluted with ethylacetate and washed with water (1×), brine (1×), dried over magnesium sulfate and evaporated. Resuspension and precipitation in ether is followed by crystallization and filtration in ethanol to afford the activated acetimidate library.

Step 3. To a cold solution (−10° C.) of the acetimidate library (1.0 equivalents) and compound 11 or 12 (3.0 equivalents) in methylene chloride (0.01 M), is added boron trifluoride etherate (3.5 equivalents). The reaction mixture is allowed to warm up slowly to room temperature and stirring is continued overnight. The reaction mixture is next quenched with sodium bicarbonate (3.5 equivalents) and diluted with ethylacetate and washed with water (1×), brine (1×), dried over magnesium sulfate and evaporated. Resuspension and precipitation in ether is followed by crystallization and filtration in ethanol to afford the desired library 2. For related art on coupling two sugars with PEG supports see Krepinsky et al., J. Am. Chem Soc., suppl. material, 113:5095, 1991.

Split Synthesis Strategy Using PEG Supports.

After each step of the synthesis, a portion of each PEG supported library is then saved and catalogued, the remaining fraction is combined, mixed and redivided. The steps of (i) coupling, (ii) saving and cataloging, and (iii) randomizing are repeated until the desired library is obtained. For a review on this recursive doconvolution strategy, see Janda, K. et al., Proc. Natl. Acad. Sci., 91:11422, 1994.

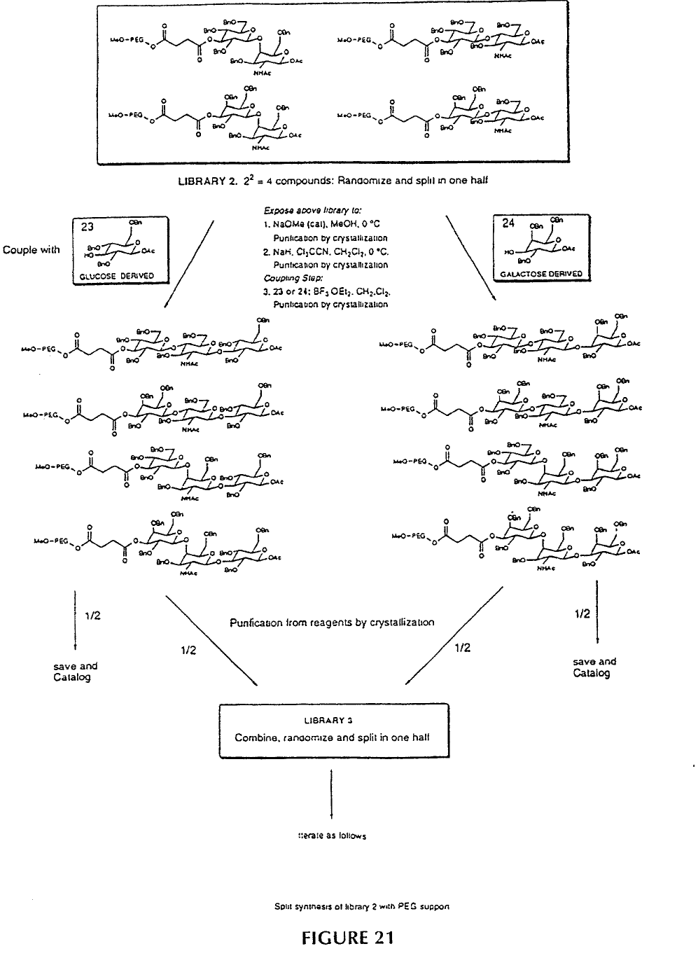

Preparation of Carbohydrate Library 3 (See FIG. 21).

Step 1. To a solution of previously randomized library 2 in MeOH (0.10 M), is added a catalytic amount of NaOCH₃ (0.10 equivalents) and the mixture is stirred for 1 hour at 0° C. or until lactol formation is complete by thin layer chromatography. Reaction mixture is then quenched with ammonium chloride (1.0 M) and then diluted with ethyl acetate (0.05 M) and washed with water (1×), brine (1×) and condensed. Crude compound is then precipitated with ethyl ether and filtered. The PEG conjugate is allowed to crystallize out from hot ethanol, filtered and washed with cold ethanol to afford the desired intermediate lactol library.

Step 2. The intermediate lactol library is then converted to the acetimidate in a solution of methylene chloride (0.10 Molar) at 0° C. The mixture is next exposed to sodium hydride (1.2 equivalents) and allowed to stir for 1 hour and then trichloroacetonitrile (1.2 equivalents) is added in one step. After an additional hour (or until the reaction appears complete i.e. thin layer chromatography), the reaction mixture is quenched with a saturated solution of sodium bicarbonate (1.0 M) and then diluted with ethylacetate and washed with water (1×), brine (1×), dried over magnesium sulfate and evaporated. Resuspension and precipitation in ether is followed by crystallization and filtration in ethanol to afford the activated acetimidate library.

Step 3. To a cold solution (−10° C.) of the acetimidate library (1.0 equivalents) and compound 23 or 24 (3.0 equivalents) in methylene chloride (0.01 M), is added boron trifluoride etherate (3.5 equivalents). The reaction mixture is allowed to warm up slowly to room temperature and stirring is continued overnight. The reaction mixture is next quenched with sodium bicarbonate (3.5 equivalents) and diluted with ethylacetate and washed with water (1×), brine (1×), dried over magnesium sulfate and evaporated. Resuspension and precipitation in ether is followed by crystallization and filtration in ethanol to afford the desired library 3. For related art on coupling two sugars with PEG supports see Krepinsky et al., J. Am. Chem Soc., suppl. material, 113:5095, 1991.

Split Synthesis Strategy Using PEG Supports.

After each step of the synthesis, a portion of each PEG supported library is then saved and catalogued, the remaining fraction is combined, mixed and redivided. The steps of (i) coupling, (ii) saving and cataloging, and (iii) randomizing are repeated until the desired library is obtained. For a review on this recursive doconvolution strategy, see Janda, K. et al., Proc. Natl. Acad. Sci. 91:11422, 1994.

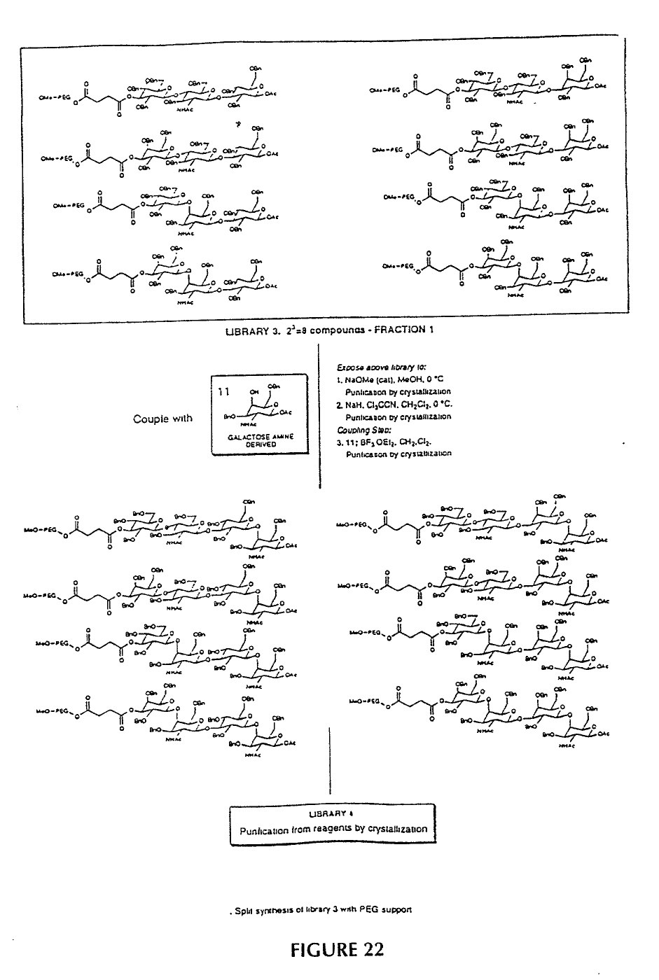

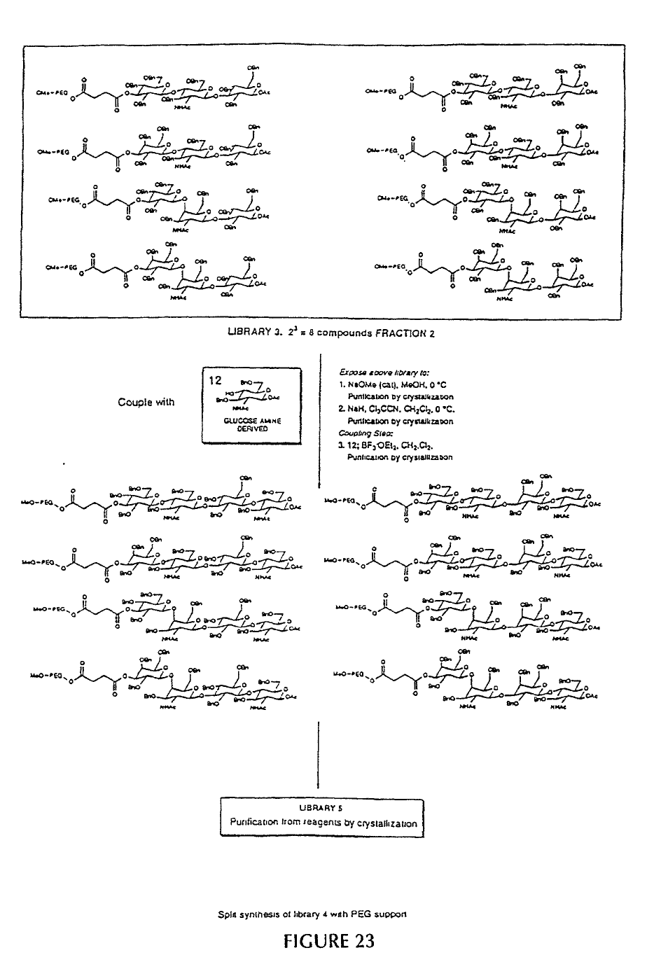

Preparation of Carbohydrate Library 4 or 5 (See FIG. 22 and FIG. 23).

Step 1. To a fraction of library 3 in MeOH (0.10 M), is added a catalytic amount of NaOCH₃ (0.10 equivalents) and the mixture is stirred for 1 hour at 0° C. or until lactol formation is complete by thin layer chromatography. Reaction mixture is then quenched with ammonium chloride (1.0 M) and then diluted with ethyl acetate (0.05 M) and washed with water (1×), brine (1×) and condensed. Crude compound is then precipitated with ethyl ether and filtered. The PEG conjugate is allowed to crystallize out from hot ethanol, filtered and washed with cold ethanol to afford the desired intermediate lactol library.

Step 2. The intermediate lactol library is then converted to the acetimidate in a solution of methylene chloride (0.10 Molar) at 0° C. The mixture is next exposed to sodium hydride (1.2 equivalents) and allowed to stir for 1 hour and then trichloroacetonitrile (1.2 equivalents) is added in one step. After an additional hour (or until the reaction appears complete i.e. thin layer chromatography), the reaction mixture is quenched with a saturated solution of sodium bicarbonate (1.0 M) and then diluted with ethylacetate and washed with water (1×), brine (1×), dried over magnesium sulfate and evaporated. Resuspension and precipitation in ether is followed by crystallization and filtration in ethanol to afford the activated acetimidate library.

Step 3. To a cold solution (−10° C.) of the acetimidate library (1.0 equivalents) and compound 11 or 12 (3.0 equivalents) in methylene chloride (0.01 M), is added boron trifluoride etherate (3.5 equivalents). The reaction mixture is allowed to warm up slowly to room temperature and stirring is continued overnight. The reaction mixture is next quenched with sodium bicarbonate (3.5 equivalents) and diluted with ethylacetate and washed with water (1×), brine (1×), dried over magnesium sulfate and evaporated. Resuspension and precipitation in ether is followed by crystallization and filtration in ethanol to afford the desired library 4 or 5. For related art on coupling two sugars with PEG supports see Krepinsky et al., J. Am. Chem Soc., suppl. material, 113:5095, 1991.

Preparation of Deprotected Libraries 4 or 5.

To a solution of benzyl protected library 4 or 5 (1.0 equiv.) in dry ethanol (0.10 M) is added 10% Pd/C (0.2 equiv.). The reaction mixture is capped with a hydrogen balloon and then allowed to stir for 1 day at 25° C. Upon completion, the reaction is filtered through a 5 cm. diameter column and filled with a plug of cotton, 0.5 cm. (length) sand, 5 cm. silica, 1 cm. celite in this order, to remove the residual carbon. Next, the column is flushed four times with ethylacetate and the solvent is evaporated. If necessary, the compound is further purified by resuspension and precipitation in ether, followed by crystallization and filtration in ethanol to afford the desired deprotected (debenzylated) library 4 or 5.

Cleavage of the Oligosaccharide from the PEG Support.

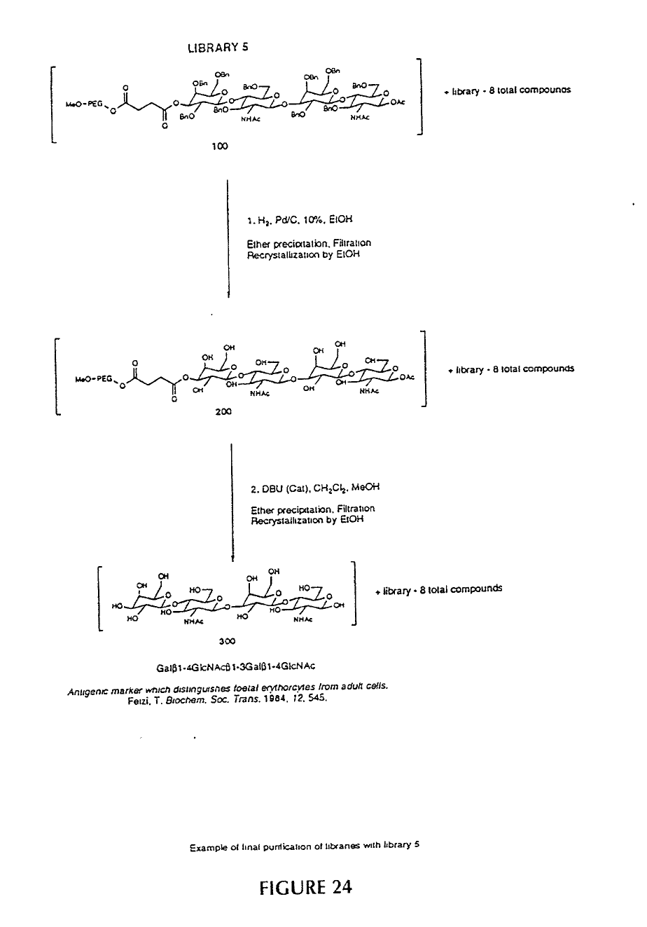

The oligosaccharide moiety is removed from the polymer by overnight treatment of library 5 (e.g., compound 200, see FIG. 24) dissolved in methylene chloride (0.5 M) with methanol (2.0 M) and 1,8-diazabicyclo[5.4.0]undec-7-ene (DBU (1 drop per 0.02 mmol of library)) with stirring at 25° C. The PEG support and deprotected oligosaccharide library are then precipitated with ether and removed by filtration. The precipitate containing both the PEG and oligosaccharide library is dissolved in hot ethanol, the PEG is allowed to crystallize out, filtered and washed with cold ethanol. The combined filtrate and washings are then evaporated to afford the fully deprotected oligosaccharide 300 with the corresponding deprotected library 5 which can be further purified by standard reverse phase HPLC analysis. For related art on PEG supports with oligosaccharides, see Krepinsky et al., J. Am. Chem Soc., suppl. material, 113:5095, 1991.

Protection of the Core Nucleotides.

Nucleotides are shown protected with common protecting groups. For the 5′-hydroxyl group, the DMTr (dimethoxytrityl) group is the favored protecting group due to its acid lability. As an alternative, the 9-phenylxanthen-9-yl (pixyl) group is perfectly acceptable and offers additional crystallinity as well as others reviewed by Beaucage et al., Tetrahedron 12:2223, 1992. The standard benzoyl and isobutyryl groups are used for the protection of the N⁴ and N²-exocyclic amino groups of 2′-deoxycytidine and 2′-deoxyguanosine, respectively. The di-n-butylaminomethylene or the standard benzoyl groups can be used at the N⁶ position of adenine with good results in most syntheses of at least 20 bases in length.

Preparation of 5′-O-DMT-N-^{3′} OPEG.

The protected 5′-O-nucleotide is coupled to PEG (preferably the monomethylether, 5000 M.W., hydroxyl number 0.20 meq/g from Aldrich, Fluka or Sigma chemical companies) via a succinyl linkage, although other functionalities such as ether and amide linkages are suitable as the linker between the PEG soluble support and the nucleotide core molecules. Monomethylether-PEG succinate coupling to the nucleotide is accomplished via standard activated ester procedures employing DCC conditions as developed by Keck et al., J. Org. Chem. 50:2394, 1985. As an alternative to linking the soluble support to the 5′-O position, the exocyclic amine groups on adenine, guanine and cytosine can be anchored to the PEG via an amide functionality as demonstrated by Napoli, et al., Nucleosides and Nucleotides, 12:21-30, 1993.

A procedure is as follows: protected aminoacid (1.0 equiv.), succinic anhydride (5 equiv.) and DMAP (1 equiv) are stirred in dry pyridine (0.20 M) at room temperature. After completion of the reaction, pyridine is removed by evaporation and the residue is subjected to flash chromatography in ethyl acetate. Next, the monomethylether of PEG (0.8 eq.) is mixed with the 3-O-hemisuccinate and dried overnight at high vacuum over the drying agent phosphorous pentoxide P₂O₅. This mixture is then dissolved in anhydrous methylene chloride (0.5 M) and a catalytic amount of dimethylaminopyridine—DMAP (0.1 equiv.) is followed by dicylcohexylcarbodiimide—DCC (0.8 equiv.) After 15 minutes, the reaction becomes cloudy and is stirred overnight at room temperature. The precipitated urea is removed by filtration, washed with methylene chloride and the volume of the combined filtrates is reduced to its original size. It is cooled to 0° C., anhydrous ether is added with vigorous stirring, and precipitated out. After filtration the solid is dissolved in hot absolute ethanol, and recrystallized to afford 27 or 28 (Procedure from Krepinsky et al., J. Am. Chem. Soc. 113:5095, 1991, supplementary materials).

Alternatives to PEG.

Although PEG (polyethylene glycol) is the soluble support of choice, due to its ease of precipitation and crystallization properties, alternative polymeric groups can be used and include polyvinyl alcohol and polyvinylamine copolymerized with polyvinylpyrrolidone. (Bayer et al., Nature 237:512, 1972)

Chain Assembly Steps 1-2.

The oligodeoxyribonucleotides are synthesised using standard conditions (Gait et al., Oligonucleotide synthesis, a practical approach IRL Press LTD, Oxford, 1984 pg. 83-115) from the 3′ end to the 5′ end in a cyclic process involving two chemical reactions per cycle. The first step removes the 5′ protecting group (DMTr or Px) with a suitable protic acid, (eg. 3%-10% (w/v) dichloroacetic acid in 1,2 dichloroethane) followed by appropriate washes to remove residual acid and precipitation or crystallization of the PEG conjugate (if necessary). Next, the appropriately protected phosphoramidite, phosphite or phosphotriester nucleotide (monomer used depends on which coupling method chosen) is condensed in the presence of a coupling agent (eg. 1-mesitylenesulphonyl-3-nitro-1,2,4-triazole—if necessary; depends on coupling method chosen) with the free 5′-hydroxyl group of the deoxyribonucleoside bound to the PEG support. For complete removal of reagents, the PEG-bound seqeunce can then be recrystallized from methylene chloride/diethyl ether or ethyl alcohol. The PEG-5000 monomethyl ether precipitates have been reported to be clean and easily filtrable, at least up to the octamer level—maximum length tested (Bonora et al., Nucleosides and Nucleotides, 10:269, 1991). Note: the purification of the PEG-bound oligomer can also be enhanced by treatment with a capping mixture before the detritylation step (eg. acetic anhydride).

Split Synthesis Strategy Using PEG Supports with Nucleotides.

After each step of the synthesis, a portion of each PEG supported library is then saved and catalogued, the remaining fraction is combined, mixed and redivided. The steps of (1-2) deprotection, coupling, (3) saving and cataloging, and (4) randomizing are repeated until the desired library is obtained. For a review on this recursive doconvolution strategy, see Janda, K. et al., Proc. Natl. Acad. Sci., 91:11422., 1994.

Purification, Oxidation, Deprotection Steps 3-6. Oxidation of the Phosphite or Phosphoramidite to the Phosphate.

The oxidation of the phosphites is accomplished via standard conditions in a solution of tetrahydrofuran-2,6-lutidine-water (2:1:1) (0.1 M total) containing 0.2 M iodine (1.1 equivalents) (I₂) at 25° C. for 10 minutes. The mixture is then washed with a saturated solutions of sodium bisulfate to remove remaining iodine (1×), sodium bicarbonate to remove residual base (1×), brine and diluted with ether to precipitate the PEG polymer. Subsequent recrystallization in hot ethanol, followed with cold ethanol washes affords the desired phosphate. For synthesis of deoxyoligonucleotides on a polymer support outlining standard methodologies see Caruthers et al., J. Am. Chem. Soc. 103:3185-3191, 1981; Gait et al., Oligonucleotide Synthesis, a practical approach IRL Press LTD. Oxford, Chapt. 4, 83, 1984.

Removal of the N²,N⁴,N⁶ and 5′ Protecting Groups.

The standard procedure of heating to approx 60° C. for 15-20 hours with benzoyl and isobutyryl groups is used for the deprotection of the N⁴ and N²-exocyclic amino groups of 2′-deoxycytidine and 2′-deoxyguanosine, and the di-n-butylaminomethylene groups at the N⁶ position of adenine.

If the 5′ DMT protecting group is used, it can be removed via treatment of the PEG bound oligonucleotide with a saturated solution of ZnBr₂ (1.5 equiv.) in nitromethane (0.1 M). The next step is a hydrolytic wash with n-butanol in tetrahydrofuran and 2,6-lutidine (all reagents available from Aldrich chemical company). Alternatively, 80% acetic acid can be used. Crystallization of the PEG support therefore affords the purified 5′ deprotected oligonucleotide.

Cleavage of Succinate Ester to Remove PEG from Oligonucleotides.

The oligonucleotide moiety is removed from the polymer by overnight treatment of the library dissolved in methylene chloride (0.5 M) with methanol (2.0 M) and 1,8-diazabicyclo[5.4.0]undec-7-ene (DBU (1 drop per 0.02 mmol of library)) with stirring at 25° C. The PEG support and deprotected oligonucleotide library are then precipitated with ether and removed by filtration. The precipitate containing both the PEG and oligonucleotide library is dissolved in hot ethanol, the PEG is allowed to crystallize out, filtered and washed with cold ethanol. The combined filtrate and washings are then evaporated to afford the fully deprotected oligonucleotide library which can be further purified by standard reverse phase HPLC analysis or ionic exchange chromatography. For related art on PEG supports with, see Bonora et al., Nucleosides and Nucleotides, 12:21, 1993.

What is claimed is:

1. A soluble combinatorial library comprised of a set of core molecules or assemblage of core molecules wherein each said core molecule or said assemblage of core molecules is attached to a soluble polymeric compound.

2. The soluble combinatorial library of claim 1 wherein the molecules in said set of core molecules or said related assemblage of molecules differ by one or more chemical moieties.

3. The soluble combinatorial library of claim 1 wherein said soluble polymeric compound is selected from the group consisting of PEG, polyvinyl alcohol and polyvinylamine copolymerized with polyvinylpyrrolidone.

4. The soluble combinatorial library of claim 1 wherein said set of core molecules comprises α-azetide compositions.

5. The soluble combinatorial library of claim 1 wherein said set of core molecules comprises triazine dione compositions.

6. The soluble combinatorial library of claim 1 wherein said set of core molecules comprises γ-lactamtide compositions.

7. The soluble combinatorial library of claim 1 wherein said set of core molecules comprises δ-lactamthiotide compositions.

8. The soluble combinatorial library of claim 1 wherein said set of core molecules comprises β-lactam nucleus containing compositions.

9. The soluble combinatorial library of claim 1 wherein said set of core molecules comprises lycoramine alkaloid nucleus containing compositions.

10. The soluble combinatorial library of claim 1 wherein said set of core molecules comprises β-blocker nucleus compositions.

11. A soluble combinatorial library comprising a collection of α-azetide compositions.

12. A soluble combinatorial library comprising a collection of triazine dione compositions.

13. A soluble combinatorial library comprising collection of γ-lactamtide compositions.

14. A soluble combinatorial library comprising a collection of δ-lactamthiotide compositions.

15. A soluble combinatorial library comprising a collection of β-lactam nucleus containing compositions.

16. A soluble combinatorial library comprising a collection of lycoramine alkaloid nucleus containing compositions.

17. A soluble combinatorial library comprising a collection of β-blocker nucleus compositions.

18. A method of generating a soluble combinatorial library comprising the step of providing a core molecule bonded to a soluble polymeric compound.

19. A method of generating a soluble combinatorial library wherein a collection of molecules is efficiently generated varying in composition by the random attachment of a core molecule at a particular position in a series of core molecules wherein said first core molecule of each of said collection of core molecules is affixed to a soluble polymeric compound.

20. A method of generating a soluble combinatorial library comprising the step of synthesizing a set of core molecules wherein each core molecule taking part in said synthesis step is dissolved in solution.

21. A method of generating a soluble combinatorial library comprising the step of performing a split synthesis wherein said split synthesis is performed in solution.

22. An improved method for generating a library of combinatorial molecules, the method employing at least two cycles of parallel split synthesis incorporating the following sequence of steps:
Step A: collecting and mixing macromolecular supports within in a common pool, each macromolecular supports having a nascent combinatorial molecule attached thereto; then
Step B: splitting and transferring the common pool of macromolecular supports of said Step A into a series of separate reaction vessels; then
Step C: elongating the nascent combinatorial molecules attached to the macromolecular supports within each separate reaction vessel of said Step B by an addition of reactants thereto; then
Step D: washing the macromolecular supports after said Step C for removing reactants therefrom; and then
repeating said Steps A, B, C, and D as desired for generating the library of combinatorial molecules;
wherein the improvement is characterized as follows:
in said Steps A, B, C, and D, the macromolecular supports are biphasic;
in said Step C: elongation of the nascent combinatorial molecules is facilitated by employment of a first solvent which renders the macromolecular supports soluble therein;
in said Step D: washing of the macromolecular supports and removal of the reactants therefrom is facilitated by employment of a second solvent which renders the macromolecular supports insoluble therein.

23. An improved method for generating a library of combinatorial molecules as described in claim 22, wherein the improvement is further characterized as follows:
in said Step C: the macromolecular supports are selected from the group consisting of polyethylene glycol (PEG), polyvinylalcohol, polyvinylamine copolymerized with polyvinyl pyrrolidine, and derivatives thereof.

24. An improved method for generating a library of combinatorial molecules as described in claim 23, wherein the improvement is further characterized as follows:
in said Step C: the macromolecular supports include polyethylene glycol (PEG).

25. An improved method for generating a library of combinatorial molecules as described in claim 23, wherein the improvement is further characterized as follows:
in said Step C: the first solvent includes an alcohol; and
in said Step D: the second solvent includes an ether.

26. An improved method for generating a library of combinatorial moleculesas described in claim 22, wherein the combinatorial molecules of the library are selected from the group consisting of oligopeptides, oligosaccharides, oligonucleotides, arylsulfonamides, and derivatives thereof and wherein the improvement is further characterized as follows:
in said Step C: the first solvent includes an alcohol and the macromolecular supports are selected from the group consisting of polyethylene glycol (PEG), polyvinylalcohol, polyvinylamine copolymerized with polyvinyl pyrrolidine, and derivatives thereof; and
in said Step D: the second solvent includes an ether.

27. An improved method for generating a library of combinatorial molecules together with a deconvolution assemblage, the method employing at least two cycles of a parallel split synthesis incorporating the following sequence of steps:
Step A: collecting and mixing macromolecular supports within a common pool, each macromolecular support having a nascent combinatorial molecule attached thereto; then
Step B: spliting and transferring the common pool of macromolecular supports of said Step A into a series of separate reaction vessels; then
Step C: elongating nascent combinatorial molecules attached to the macromolecular supports within each separate reaction vessel of said Step B by an addition of reactants thereto; then
Step D: washing the macromolecular supports after said Step C for removing reactants therefrom; then
Step E: removing an aliquot of the macromolecular supports from each reaction vessel after said Step D for forming the deconvolution assemblage; and then
repeating said Steps A, B, C, D, and E as desired for generating the library of combinatorial molecules;
wherein the improvement comprises:
in said Steps A, B, C, D, and E, the macromolecular support is biphasic;
in said Steps C: elongation of the nascent combinatorial molecules is facilitated by employment of a first solvent which renders the macromolecular supports soluble therein;
in said Step D: washing of the macromolecular supports and removal of the reactants therefrom is facilitated by employment of a second solvent which renders the macromolecular supports insoluble therein.

28. An improved method for generating a library of combinatorial molecules as described in claim 27, wherein the improvement is further characterized as follows:
in said Step C: the macromolecular supports are selected from the group consisting of polyethylene glycol (PEG), polyvinylalcohol, polyvinylamine copolymerized with polyvinyl pyrrolidine, and derivatives thereof.

29. An improved method for generating a library of combinatorial molecules as described in claim 28, wherein the improvement is further characterized as follows:
in said Step C: the macromolecular supports include polyethylene glycol (PEG).

30. An improved method for generating a library of combinatorial molecules as described in claim 28, wherein the improvement is further characterized as follows:
in said Step C: the first solvent includes an alcohol; and
in said Step D: the second solvent includes an ether.

31. An improved method for generating a library of combinatorial molecules as described in claim 27, wherein the combinatorial molecules of the library are selected from the group consisting of oligopeptides, oligosaccharides, oligonucleotides, arylsulfonamides, and derivatives thereof and wherein the improvement is further characterized as follows:
in said Step C: the first solvent includes an alcohol and the macromolecular supports are selected from the group consisting of polyethylene glycol (PEG), polyvinylalcohol, polyvinylamine copolymerized with polyvinyl pyrrolidine, and derivatives thereof; and
in said Step D: the second solvent includes an ether.

32. A method for generating a library of combinatorial molecules the method employing at least two cycles of a parallel split synthesis incorporating the following sequence of steps:
Step A: collecting and mixing biphasic biphasic macromolecular supports within a common pool, each biphasic macromolecular support having a nascent combinatorial molecule attached thereto; then
Step B: splitting and transferring the common pool of biphasic macromolecular supports of said Step A into a series of separate reaction vessels; then
Step C: elongating the nascent combinatorial molecules attached to the biphasic macromolecular supports within each separate reaction vessel of said Step B by an addition of reactants thereto in the presence of a first solvent which renders the biphasic macromolecular supports soluble therein; then
Step D: washing the biphasic macromolecular supports after said Step C for removing reactants therefrom in the presence of a second solvent which renders the biphasic macromolecular supports insoluble therein;
repeating said Steps A, B, C, and D as desired for generating the library of combinatorial molecules.

33. A method for generating a library of combinatorial molecules as described in claim 31, further characterized as follows:
in said Step C: the biphasic macromolecular supports are selected from the group consisting of polyethylene glycol (PEG), polyvinylalcohol, polyvinylamine copolymerized with polyvinyl pyrrolidine, and derivatives thereof.

34. A method for generating a library of combinatorial molecules as described in claim 33, further characterized as follows:
in said Step C: the biphasic macromolecular supports include polyethylene glycol (PEG).

35. A method for generating a library of combinatorial molecules as described in claim 33, further characterized as follows:
in said Step C: the first solvent includes an alcohol; and
in said Step D: the second solvent includes an ether.

36. A method for generating a library of combinatorial molecules as described in claim 32, wherein the combinatorial molecules of the library are selected from the group consisting of oligopeptides, oligosaccharides, oligonucleotides, arylsulfonamides, and derivatives thereof and wherein the method is further characterized as follows:
in said Step C: the first solvent includes an alcohol and the macromolecular supports are selected from the group consisting of polyethylene glycol (PEG), polyvinylalcohol, polyvinylamine copolymerized with polyvinyl pyrrolidine, and derivatives thereof; and
in said Step D: the second solvent includes an ether.

37. A method for generating a library of combinatorial molecules as described in claim 32, further comprisingg the following additional step:
Step E: after said Step D, removing an aliquot of the macromolecular supports from the reaction vessels for forming a deconvolution assemblage.

38. A method of generating a combinatorial library comprising a collection of α-azetide compositions.
